SEQUENCE LISTING (175 sequences)

SEQ ID NO: 1 - protein, 876 aa, synthetic construct
  REGION 1..876: note = An artificially synthesized sequence
MGWSCIILFLVATATGVHSTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGFCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQGGGGSAPSTCSKPMCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPTVLDSDGSYFLYSKLSVPTSEWQRGDVFTCSVMHEALHNHYTQKSISRSPGK

SEQ ID NO: 2 - protein, 656 aa, synthetic construct
  REGION 1..656: note = An artificially synthesized sequence
MGWSCIILFLVATATGVHSTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGFCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQGGGGSDYKDDDDK

SEQ ID NO: 3 - protein, 678 aa, synthetic construct
  REGION 1..678: note = An artificially synthesized sequence
MGWSCIILFLVATATGVHSTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQGGGGSSAWSHPQFEKGGGSGGGSGGSAWSHPQFEK

SEQ ID NO: 4 - skipped

SEQ ID NO: 5 - protein, 115 aa, synthetic construct
  REGION 1..115: note = An artificially synthesized sequence
QSVEESGGRLVTPGTPLTLTCTVSGFSLSTYDMSWVRQAPGKGLEWIGYIYAGSIGTYYASWVNGRFTISKTSTTMDLRMTSLTAADTATYFCARGGAGASFDFWGPGTLVTVSS

SEQ ID NO: 6 - protein, 116 aa, synthetic construct
  REGION 1..116: note = An artificially synthesized sequence
QSLEESGGRLVTPGTPLTLTCKASGFSLSSYAMIWVRQAPGKGLESIGMVYIHSGGTYYASWAKGRFTISKTSTTVDLKITSPTTEDTATYFCARYNGGGSGTDLWGPGTLVTVSS

SEQ ID NO: 7 - protein, 125 aa, synthetic construct
  REGION 1..125: note = An artificially synthesized sequence
QSVEESGGRLVTPGTPLTLTCTVSGFSLSRYAMSWVRQAPGKGLEWIGIIVSSSGSTYYATWATGRFTISKTSTTVDLKITSPTTEDTATYFCARIRGYDNIGYGYPWFYYFNLWGPGTLVTVSS

SEQ ID NO: 8 - protein, 122 aa, synthetic construct
  REGION 1..122: note = An artificially synthesized sequence
QSVEESGGRLVTPGTPLTLTCTVSGFSLSSNAISWVRQAPGKGLEYIGIISSSGSTYYASWAKGRFTISKTSTTVDLKITSPTTEDTATYFCARGVDIYGHAYATPYGMDLWGPGTLVTVSS

SEQ ID NO: 9 - protein, 117 aa, synthetic construct
  REGION 1..117: note = An artificially synthesized sequence
QSVEESGGRLVTPGTPLTLTCTVSGFSLSNYDMTWVRQTPGKGLEWIGYISSSGSTSYASWAKGRFTISKTSTTVDLKMTSLTTEDTATYFCARDGSSSDYDRLDLWGQGTLVTVSS

SEQ ID NO: 10 - protein, 122 aa, synthetic construct
  REGION 1..122: note = An artificially synthesized sequence
QSVEESGGRLVTPGTPLTLTCTVSGFSLSSNAMGWVRQAPGKGLEYIGIISSSGSTYYASWAKGRFTISKTSTTVDLKITSPTTEDTATYFCARGVDIYGHAYATPYGMDLWGPGTLVTVSS

SEQ ID NO: 11 - protein, 123 aa, synthetic construct
  REGION 1..123: note = An artificially synthesized sequence
QSVEESGGRLVTPGTPLTLTCTVSGFSLSSNAISWVRQAPGKGLEYIGIISSSGSTYYANWAKGRFTISKTSSTTVDLKMTSATTEDTATYFCARGVDIYGHAYATPYGMDLWGPGTLVTVSS

SEQ ID NO: 12 - protein, 116 aa, synthetic construct
  REGION 1..116: note = An artificially synthesized sequence
QSLEESGGRLVTPGTPLTLTCKASGFSLSSYAMIWVRQAPGKGLESIGMVYIHSGGTYYASWAKGRFTISKTSTTVDLKITSPTTEDTATYFCAKYNGGGSGTDLWGPGTLVTVSS

SEQ ID NO: 13 - protein, 122 aa, synthetic construct; identical to SEQ ID NO: 10
  REGION 1..122: note = An artificially synthesized sequence

SEQ ID NO: 14 - protein, 122 aa, synthetic construct
  REGION 1..122: note = An artificially synthesized sequence
QSVEESGGRLVAPGTPLTLTCTVSGIDLSSDAMAWVRQAPGKGLEYIGIISSSGSTYYASWAKGRFTISKTSTTVDLKITSPTTEDTATYFCARGVDIYGHAYATPYGMDLWGPGTLVTVSS

SEQ ID NO: 15 - protein, 122 aa, synthetic construct
  REGION 1..122: note = An artificially synthesized sequence
QSVEESGGRLVTPGTPLTLTCTVSGFSLSSYAMGWVRQAPGKGLEYIGIISSSGSTYYANWAKGRFTISKTSTTVDLKITSPTTEDTATYFCARGVDIYGHPYATPYGMDLWGPGTLVTVSS

SEQ ID NO: 16 - protein, 122 aa, synthetic construct
  REGION 1..122: note = An artificially synthesized sequence
QSVEESGGRLVTPGTPLTLTCTVSGFSLSSNAISWVRQAPGKGLQYIGIISSNGITYYASWAKGRFTISKTSTTVDLKITSPTTEDTATYFCARGVDIYGHAYATPYGMDLWGPGTLVTVSS

SEQ ID NO: 17 - protein, 110 aa, synthetic construct
  REGION 1..110: note = An artificially synthesized sequence
ARVLTQTPSPVSAAVGGTVTITCQASQSVYSNNLLSWYQQKPGQPPKLLIYYAATLASGVPSRFKGSGSGTQFTLTISDIQCDDAATYYCQGYFSGGVYTFGGGTKVEIK

SEQ ID NO: 18 - protein, 112 aa, synthetic construct
  REGION 1..112: note = An artificially synthesized sequence
DIVMTQTPSSVSEPVGGTVTIKCQASQSIYSYLSWYQQKPGQRPKLLIYYASTLASGVPSRFKGSGSGTQFTLTISDLECADAATYYCQNNYGSDSNIYGIAFGGGTKVEIK

SEQ ID NO: 19 - protein, 112 aa, synthetic construct
  REGION 1..112: note = An artificially synthesized sequence
DVVMTQTPASVEAAVGGTVTIKCQASQSISSYLAWYQQKPGQPPKLLIYDASDLASGVPSRFKGSGSGTQFTLTISDLECADAATYYCQSYYYSSSSSYGFAFGGGTKVEIK

SEQ ID NO: 20 - protein, 112 aa, synthetic construct
  REGION 1..112: note = An artificially synthesized sequence
EVVMTQTPASVSEPVGGTVTIKCQASQSISSYLAWYQQKPGQPPKRLIYYASTLASGVPSRFKGSGSGTEFTLTISDLECDDAATYYCQSNYGSSSSSYEDTFGGGTKVEIK

SEQ ID NO: 21 - protein, 110 aa, synthetic construct
  REGION 1..110: note = An artificially synthesized sequence
AYDMTQTPASVEVAVGGTVTIKCQASQNIYSFLAWYQQKPGQPPKLLIYTAFYLASGVPSRFKGSGSGTEYTLTISGVECADAATYYCQQGYSRSNVDNTFGGGTKVEIK

SEQ ID NO: 22 - protein, 112 aa, synthetic construct
  REGION 1..112: note = An artificially synthesized sequence
EVVMTQTPASVSEPVGGTVTIKCQASQSISSYLSWYQQKPGQPPKRLIYYSSTLASGVPSRFKGSGSGTEFTLTISDLECDDAATYYCQSNYGSSSSSYEDTFGGGTKVEIK

SEQ ID NO: 23 - protein, 112 aa, synthetic construct
  REGION 1..112: note = An artificially synthesized sequence
EVVMTQTPASVSEPVGGTVTIKCQASQSISSYLSWYQQKPGQPPKLLIYYSSTLASGVPSRFKGSGSGTEFTLTISDLECADAATYYCQSNYGSSSSSYEDTFGGGTKVEIK

SEQ ID NO: 24 - protein, 112 aa, synthetic construct; identical to SEQ ID NO: 18
  REGION 1..112: note = An artificially synthesized sequence

SEQ ID NO: 25 - protein, 112 aa, synthetic construct; identical to SEQ ID NO: 22
  REGION 1..112: note = An artificially synthesized sequence

SEQ ID NO: 26 - protein, 112 aa, synthetic construct
  REGION 1..112: note = An artificially synthesized sequence
EVVMTQTPASVSEPVGGTVTIKCQASQSISSYLSWYQQKPGQPPKLLIYYASTLASGVPSRFKGSGSGTEFTLTISDLECDDAATYYCQSNYGSSSSSYEDTFGGGTKVEIK

SEQ ID NO: 27 - protein, 112 aa, synthetic construct
  REGION 1..112: note = An artificially synthesized sequence
EVVMTQTPASVEVAVGGTVTIKCQASQSISSYLAWYQQKPGQPPKLLIYYASTLASGVPSRFKGSGSGTEFTLTISDLECDDAATYYCQSNYGSSSSSYEDTFGGGTKVEIK

SEQ ID NO: 28 - protein, 112 aa, synthetic construct
  REGION 1..112: note = An artificially synthesized sequence
EVVMTQTPASVSEPVGGTVTIKCQASQSISSYLSWYQQKPGQPPKRLIYYASTLASGVPSRFKGSGSGTEFTLTISDLECDDAATYYCQSNYGSSSSSYEDTFGGGTKVEIK

SEQ ID NO: 29 - protein, 5 aa, synthetic construct
  REGION 1..5: note = An artificially synthesized sequence
TYDMS

SEQ ID NO: 30 - protein, 5 aa, synthetic construct
  REGION 1..5: note = An artificially synthesized sequence
SYAMI

SEQ ID NO: 31 - protein, 5 aa, synthetic construct
  REGION 1..5: note = An artificially synthesized sequence
RYAMS

SEQ ID NO: 32 - protein, 5 aa, synthetic construct
  REGION 1..5: note = An artificially synthesized sequence
SNAIS

SEQ ID NO: 33 - protein, 5 aa, synthetic construct
  REGION 1..5: note = An artificially synthesized sequence
NYDMT

SEQ ID NO: 34 - protein, 5 aa, synthetic construct
  REGION 1..5: note = An artificially synthesized sequence
SNAMG

SEQ ID NO: 35 - protein, 5 aa, synthetic construct
  REGION 1..5: note = An artificially synthesized sequence
SNAIS

SEQ ID NO: 36 - protein, 5 aa, synthetic construct
  REGION 1..5: note = An artificially synthesized sequence
SYAMI

SEQ ID NO: 37 - protein, 5 aa, synthetic construct
  REGION 1..5: note = An artificially synthesized sequence
SNAMG

SEQ ID NO: 38 - protein, 5 aa, synthetic construct
  REGION 1..5: note = An artificially synthesized sequence
SDAMA

SEQ ID NO: 39 - protein, 5 aa, synthetic construct
  REGION 1..5: note = An artificially synthesized sequence
SYAMG

SEQ ID NO: 40 - protein, 5 aa, synthetic construct
  REGION 1..5: note = An artificially synthesized sequence
SNAIS

SEQ ID NO: 41 - protein, 17 aa, synthetic construct
  REGION 1..17: note = An artificially synthesized sequence
YIYAGSIGTYYASWVNG

SEQ ID NO: 42 - protein, 17 aa, synthetic construct
  REGION 1..17: note = An artificially synthesized sequence
MVYIHSGGTYYASWAKG

SEQ ID NO: 43 - protein, 17 aa, synthetic construct
  REGION 1..17: note = An artificially synthesized sequence
IIVSSSGSTYYATWATG

SEQ ID NO: 44 - protein, 16 aa, synthetic construct
  REGION 1..16: note = An artificially synthesized sequence
IISSSGSTYYASWAKG

SEQ ID NO: 45 - protein, 16 aa, synthetic construct
  REGION 1..16: note = An artificially synthesized sequence
YISSSGSTSYASWAKG

SEQ ID NO: 46 - protein, 16 aa, synthetic construct
  REGION 1..16: note = An artificially synthesized sequence
IISSSGSTYYASWAKG

SEQ ID NO: 47 - protein, 16 aa, synthetic construct
  REGION 1..16: note = An artificially synthesized sequence
IISSSGSTYYANWAKG

SEQ ID NO: 48 - protein, 17 aa, synthetic construct
  REGION 1..17: note = An artificially synthesized sequence
MVYIHSGGTYYASWAKG

SEQ ID NO: 49 - protein, 16 aa, synthetic construct
  REGION 1..16: note = An artificially synthesized sequence
IISSSGSTYYASWAKG

SEQ ID NO: 50 - protein, 16 aa, synthetic construct
  REGION 1..16: note = An artificially synthesized sequence
IISSSGSTYYASWAKG

SEQ ID NO: 51 - protein, 16 aa, synthetic construct
  REGION 1..16: note = An artificially synthesized sequence
IISSSGSTYYANWAKG

SEQ ID NO: 52 - protein, 16 aa, synthetic construct
  REGION 1..16: note = An artificially synthesized sequence
IISSNGITYYASWAKG

SEQ ID NO: 53 - protein, 9 aa, synthetic construct
  REGION 1..9: note = An artificially synthesized sequence
GGAGASFDF

SEQ ID NO: 54 - protein, 10 aa, synthetic construct
  REGION 1..10: note = An artificially synthesized sequence
YNGGGSGTDL

SEQ ID NO: 55 - protein, 19 aa, synthetic construct
  REGION 1..19: note = An artificially synthesized sequence
IRGYDNIGYGYPWFYYFNL

SEQ ID NO: 56 - protein, 17 aa, synthetic construct
  REGION 1..17: note = An artificially synthesized sequence
GVDIYGHAYATPYGMDL

SEQ ID NO: 57 - protein, 12 aa, synthetic construct
  REGION 1..12: note = An artificially synthesized sequence
DGSSSDYDRLDL

SEQ ID NO: 58 - protein, 17 aa, synthetic construct
  REGION 1..17: note = An artificially synthesized sequence
GVDIYGHAYATPYGMDL

SEQ ID NO: 59 - protein, 17 aa, synthetic construct
  REGION 1..17: note = An artificially synthesized sequence
GVDIYGHAYATPYGMDL

SEQ ID NO: 60 - protein, 10 aa, synthetic construct
  REGION 1..10: note = An artificially synthesized sequence
YNGGGSGTDL

SEQ ID NO: 61 - protein, 17 aa, synthetic construct
  REGION 1..17: note = An artificially synthesized sequence
GVDIYGHAYATPYGMDL

SEQ ID NO: 62 - protein, 17 aa, synthetic construct
  REGION 1..17: note = An artificially synthesized sequence
GVDIYGHAYATPYGMDL

SEQ ID NO: 63 - protein, 17 aa, synthetic construct
  REGION 1..17: note = An artificially synthesized sequence
GVDIYGHPYATPYGMDL

SEQ ID NO: 64 - protein, 17 aa, synthetic construct
  REGION 1..17: note = An artificially synthesized sequence
GVDIYGHAYATPYGMDL

SEQ ID NO: 65 - protein, 13 aa, synthetic construct
  REGION 1..13: note = An artificially synthesized sequence
QASQSVYSNNLLS

SEQ ID NO: 66 - protein, 11 aa, synthetic construct
  REGION 1..11: note = An artificially synthesized sequence
QASQSIYSYLS

SEQ ID NO: 67 - protein, 11 aa, synthetic construct
  REGION 1..11: note = An artificially synthesized sequence
QASQSISSYLA

SEQ ID NO: 68 - protein, 11 aa, synthetic construct
  REGION 1..11: note = An artificially synthesized sequence
QASQSISSYLA

SEQ ID NO: 69 - protein, 11 aa, synthetic construct
  REGION 1..11: note = An artificially synthesized sequence
QASQNIYSFLA

SEQ ID NO: 70 - protein, 11 aa, synthetic construct
  REGION 1..11: note = An artificially synthesized sequence
QASQSISSYLS

SEQ ID NO: 71 - protein, 11 aa, synthetic construct
  REGION 1..11: note = An artificially synthesized sequence
QASQSISSYLS

SEQ ID NO: 72 - protein, 11 aa, synthetic construct
  REGION 1..11: note = An artificially synthesized sequence
QASQSIYSYLS

SEQ ID NO: 73 - protein, 11 aa, synthetic construct
  REGION 1..11: note = An artificially synthesized sequence
QASQSISSYLS

SEQ ID NO: 74 - protein, 11 aa, synthetic construct
  REGION 1..11: note = An artificially synthesized sequence
QASQSISSYLS

SEQ ID NO: 75 - protein, 11 aa, synthetic construct
  REGION 1..11: note = An artificially synthesized sequence
QASQSISSYLA

SEQ ID NO: 76 - protein, 11 aa, synthetic construct
  REGION 1..11: note = An artificially synthesized sequence
QASQSISSYLS

SEQ ID NO: 77 - protein, 7 aa, synthetic construct
  REGION 1..7: note = An artificially synthesized sequence
YAATLAS

SEQ ID NO: 78 - protein, 7 aa, synthetic construct
  REGION 1..7: note = An artificially synthesized sequence
YASTLAS

SEQ ID NO: 79 - protein, 7 aa, synthetic construct
  REGION 1..7: note = An artificially synthesized sequence
DASDLAS

SEQ ID NO: 80 - protein, 7 aa, synthetic construct
  REGION 1..7: note = An artificially synthesized sequence
YASTLAS

SEQ ID NO: 81 - protein, 7 aa, synthetic construct
  REGION 1..7: note = An artificially synthesized sequence
TAFYLAS

SEQ ID NO: 82 - protein, 7 aa, synthetic construct
  REGION 1..7: note = An artificially synthesized sequence
YSSTLAS

SEQ ID NO: 83 - protein, 7 aa, synthetic construct
  REGION 1..7: note = An artificially synthesized sequence
YSSTLAS

SEQ ID NO: 84 - protein, 7 aa, synthetic construct
  REGION 1..7: note = An artificially synthesized sequence
YASTLAS

SEQ ID NO: 85 - protein, 7 aa, synthetic construct
  REGION 1..7: note = An artificially synthesized sequence
YSSTLAS

SEQ ID NO: 86 - protein, 7 aa, synthetic construct
  REGION 1..7: note = An artificially synthesized sequence
YASTLAS

SEQ ID NO: 87 - protein, 7 aa, synthetic construct
  REGION 1..7: note = An artificially synthesized sequence
YASTLAS

SEQ ID NO: 88 - protein, 7 aa, synthetic construct
  REGION 1..7: note = An artificially synthesized sequence
YASTLAS

SEQ ID NO: 89 - protein, 10 aa, synthetic construct
  REGION 1..10: note = An artificially synthesized sequence
QGYFSGGVYT

SEQ ID NO: 90 - protein, 14 aa, synthetic construct
  REGION 1..14: note = An artificially synthesized sequence
QNNYGSDSNIYGIA

SEQ ID NO: 91 - protein, 14 aa, synthetic construct
  REGION 1..14: note = An artificially synthesized sequence
QSYYYSSSSSYGFA

SEQ ID NO: 92 - protein, 14 aa, synthetic construct
  REGION 1..14: note = An artificially synthesized sequence
QSNYGSSSSSYEDT

SEQ ID NO: 93 - protein, 12 aa, synthetic construct
  REGION 1..12: note = An artificially synthesized sequence
QQGYSRSNVDNT

SEQ ID NO: 94 - protein, 14 aa, synthetic construct
  REGION 1..14: note = An artificially synthesized sequence
QSNYGSSSSSYEDT

SEQ ID NO: 95 - protein, 14 aa, synthetic construct
  REGION 1..14: note = An artificially synthesized sequence
QSNYGSSSSSYEDT

SEQ ID NO: 96 - protein, 14 aa, synthetic construct
  REGION 1..14: note = An artificially synthesized sequence
QNNYGSDSNIYGIA

SEQ ID NO: 97 - protein, 14 aa, synthetic construct
  REGION 1..14: note = An artificially synthesized sequence
QSNYGSSSSSYEDT

SEQ ID NO: 98 - protein, 14 aa, synthetic construct
  REGION 1..14: note = An artificially synthesized sequence
QSNYGSSSSSYEDT

SEQ ID NO: 99 - protein, 14 aa, synthetic construct
  REGION 1..14: note = An artificially synthesized sequence
QSNYGSSSSSYEDT

SEQ ID NO: 100 - protein, 14 aa, synthetic construct
  REGION 1..14: note = An artificially synthesized sequence
QSNYGSSSSSYEDT

SEQ ID NO: 101 - protein, 122 aa, synthetic construct
  REGION 1..122: note = An artificially synthesized sequence
QVQLVESGGGVVQPGGSLRLSCAASGFTFSNAWMHWVRQAPGKGLEWVAQIKDKSQNYATYVAESVKGRFTISRADSKNSIYLQMNSLKTEDTAVYYCRYVHYAAGYGVDIWGQGTTVTVSS

SEQ ID NO: 102 - protein, 112 aa, synthetic construct
  REGION 1..112: note = An artificially synthesized sequence
DIVMTQSPLSLPVTPGEPASISCRSSQPLVHSNRNTYLHWYQQKPGQAPRLLIYKVSNRFSGVPDRFSGSGSGTDFTLKISRVEAEDVGVYYCGQGTQVPYTFGQGTKLEIK

SEQ ID NO: 103 - protein, 5 aa, synthetic construct
  REGION 1..5: note = An artificially synthesized sequence
NAWMH

SEQ ID NO: 104 - protein, 19 aa, synthetic construct
  REGION 1..19: note = An artificially synthesized sequence
QIKDKSQNYATYVAESVKG

SEQ ID NO: 105 - protein, 11 aa, synthetic construct
  REGION 1..11: note = An artificially synthesized sequence
VHYAAGYGVDI

SEQ ID NO: 106 - protein, 16 aa, synthetic construct
  REGION 1..16: note = An artificially synthesized sequence
RSSQPLVHSNRNTYLH

SEQ ID NO: 107 - protein, 7 aa, synthetic construct
  REGION 1..7: note = An artificially synthesized sequence
KVSNRFS

SEQ ID NO: 108 - protein, 9 aa, synthetic construct
  REGION 1..9: note = An artificially synthesized sequence
GQGTQVPYT

SEQ ID NO: 109 - protein, 120 aa, synthetic construct [sequence omitted]

SEQ ID NO: 110 - protein, 119 aa, synthetic construct [sequence omitted]

SEQ ID NO: 111 - protein, 116 aa, synthetic construct
  REGION 1..116: note = An artificially synthesized sequence
QVQLQQSGPQLVRPGASVKISCKASGYSFTSYWMHWVNQRPGQGLEWIGMIDPSYSETRLNQKFKDKATLTVDKSSSTAYMQLSSPTSEDSAVYYCALYGNYFDYWGQGTTLTVSS

SEQ ID NO: 112 - protein, 107 aa, synthetic construct [sequence omitted]

SEQ ID NO: 113 - protein, 107 aa, synthetic construct [sequence omitted]

SEQ ID NO: 114 - protein, 107 aa, synthetic construct
  REGION 1..107: note = An artificially synthesized sequence
DIQMTQSSSSFSVSLGDRVTITCKASEDIYNRLAWYQQKPGNAPRLLISGATSLETGVPSRFSGSGSGKDYTLSITSLQTEDVATYYCQQYWSTPYTFGGGTKLEVK

SEQ ID NO: 115 - protein, 5 aa, synthetic construct
  REGION 1..5: note = An artificially synthesized sequence
DTYIH

SEQ ID NO: 116 - protein, 5 aa, synthetic construct
  REGION 1..5: note = An artificially synthesized sequence
DYTMD

SEQ ID NO: 117 - protein, 5 aa, synthetic construct
  REGION 1..5: note = An artificially synthesized sequence
SYWMH

SEQ ID NO: 118 - protein, 17 aa, synthetic construct
  REGION 1..17: note = An artificially synthesized sequence
RIYPTNGYTRYADSVKG

SEQ ID NO: 119 - protein, 17 aa, synthetic construct
  REGION 1..17: note = An artificially synthesized sequence
DVNPNSGGSIYNQRFKG

SEQ ID NO: 120 - protein, 17 aa, synthetic construct
  REGION 1..17: note = An artificially synthesized sequence
MIDPSYSETRLNQKFKD

SEQ ID NO: 121 - protein, 11 aa, synthetic construct
  REGION 1..11: note = An artificially synthesized sequence
WGGDGFYAMDY

SEQ ID NO: 122 - protein, 10 aa, synthetic construct
  REGION 1..10: note = An artificially synthesized sequence
NLGPSFYFDY

SEQ ID NO: 123 - protein, 7 aa, synthetic construct
  REGION 1..7: note = An artificially synthesized sequence
YGNYFDY

SEQ ID NO: 124 - protein, 11 aa, synthetic construct
  REGION 1..11: note = An artificially synthesized sequence
RASQDVNTAVA

SEQ ID NO: 125 - protein, 11 aa, synthetic construct
  REGION 1..11: note = An artificially synthesized sequence
KASQDVSIGVA

SEQ ID NO: 126 - protein, 11 aa, synthetic construct
  REGION 1..11: note = An artificially synthesized sequence
KASEDIYNRLA

SEQ ID NO: 127 - protein, 7 aa, synthetic construct
  REGION 1..7: note = An artificially synthesized sequence
SASFLYS

SEQ ID NO: 128 - protein, 7 aa, synthetic construct
  REGION 1..7: note = An artificially synthesized sequence
SASYRYT

SEQ ID NO: 129 - protein, 7 aa, synthetic construct
  REGION 1..7: note = An artificially synthesized sequence
GATSLET

SEQ ID NO: 130 - protein, 9 aa, synthetic construct
  REGION 1..9: note = An artificially synthesized sequence
QQHYTTPPT

SEQ ID NO: 131 - protein, 9 aa, synthetic construct
  REGION 1..9: note = An artificially synthesized sequence
QQYYIYPYT

SEQ ID NO: 132 - protein, 9 aa, synthetic construct
  REGION 1..9: note = An artificially synthesized sequence
QQYWSTPYT

SEQ ID NO: 133 - protein, 328 aa, synthetic construct
  REGION 1..328: note = An artificially synthesized sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 134 - skipped

SEQ ID NO: 135 - protein, 328 aa, synthetic construct
  REGION 1..328: note = An artificially synthesized sequence
ASTKGPSVFPLAPSSRSTSESTAALGCLVKDYFPEPVTVSWNSGALTSGVHTFPAVLQSSGLYSLSSVVTVPSSSLGTKTYTCNVDHKPSNTKVDKRVEPKSCDKTHTCPPCPAPELRRGPKVFLFPPKPKDTLMISRTPEVTCVVVDVSQEDPEVQFNWYVDGVEVHNAKTKPREEQFASTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPYLDSDGSFFLYSKLTVDKSRWQEGNVFSCSVMHEALHNHYTQESLSLSP

SEQ ID NO: 136 - protein, 107 aa, synthetic construct
  REGION 1..107: note = An artificially synthesized sequence
RTVAAPSVFIFPPSDEQLKSGTASVVCLLNNFYPREAKVQWKVDNALQSGNSQESVTEQDSKDCTYSLSSTLTLSKADYEKHKVYACEVTHQGLSSPVTKSFNRGEC

SEQ ID NO: 137 - protein, 328 aa, synthetic construct
  REGION 1..328: note = An artificially synthesized sequence
ASTKGPSVFPLAPSSRSTSESTAALGCLVKDYFPEPVTVSWNSGALTSGVHTFPAVLQSSGLYSLSSVVTVPSSSLGTKTYTCNVDHKPSNTKVDKRVEPKSCDKTHTCPPCPAPELRRGPKVFLFPPKPKDTLMISRTPEVTCVVVDVSQEDPEVQFNWYVDGVEVHNAKTKPREEQFASTYRVVSVLTVLHQDWLNGKEYKCKVSNKGLPSSIEKTISKAKGQPREPQVYTLPPSRKEMTKNQVSLTCLVKGFYPSDIAVEWESNGQPENNYKTTPPYLDSDGSFFLYSKLTVDKSRWQEGNVFSCSVMHEALHNHYTQKSLSLSP

SEQ ID NO: 138 - protein, 107 aa, synthetic construct [sequence omitted]

SEQ ID NO: 139 - protein, 112 aa, synthetic construct
  REGION 1..112: note = An artificially synthesized sequence
EVVMTQTPASVSEPVGGTVTIKCQASQSISSYLAWYQQKPGQPPKLLIYYASTLASGVPSRFKGSGSGTEFTLTISDLECDDAATYYCQSNYGSSSSSYEDTFGGGTKVEIK

SEQ ID NO: 140 - protein, 141 aa, synthetic construct
  REGION 1..141: note = An artificially synthesized sequence
DIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 141 - protein, 179 aa, synthetic construct
  REGION 1..179: note = An artificially synthesized sequence
EDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 142 - protein, 173 aa, synthetic construct
  REGION 1..173: note = An artificially synthesized sequence
DKQLDADVSPKPTIFLPSIAETKLQKAGTYLCLLEKFFPDVIKIHWQEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKS

SEQ ID NO: 143 - protein, 204 aa, synthetic construct
  REGION 1..204: note = An artificially synthesized sequence
KQLDADVSPKPTIFLPSIAETKLQKAGTYLCLLEKFFPDIIKIHWQEKKSNTILGSQEGNTMKTNDTYMKFSWLTVPEESLDKEHRCIVRHENNKNGIDQEIIFPPIKTDVTTVDPKDSYSKDANDVTTVDPKYNYSKDANDVITMDPKDNWSKDANDTLLLQLTNTSAYYMYLLLLLKSVVYFAIITCCLLGRTAFCCNGEKS

SEQ ID NO: 144 - protein, 177 aa, synthetic construct
  REGION 1..177: note = An artificially synthesized sequence
PSYTGGYADKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 145 - protein, 182 aa, synthetic construct
  REGION 1..182: note = An artificially synthesized sequence
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 146 - protein, 171 aa, synthetic construct
  REGION 1..171: note = An artificially synthesized sequence
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 147 - protein, 207 aa, synthetic construct
  REGION 1..207: note = An artificially synthesized sequence
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 148 - protein, 330 aa, synthetic construct [sequence omitted]

SEQ ID NO: 149 - protein, 326 aa, synthetic construct
  REGION 1..326: note = An artificially synthesized sequence
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 150 - protein, 377 aa, synthetic construct
  REGION 1..377: note = An artificially synthesized sequence
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 151 - protein, 327 aa, synthetic construct
  REGION 1..327: note = An artificially synthesized sequence
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 152 - protein, 374 aa, synthetic construct
  REGION 1..374: note = An artificially synthesized sequence
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHVLFYLAVGIMFLVNTVLWVTIRKELKRKKKWDLEISLDSGHEKKVISSLQEDRHLEEELKCQEQKEEQLQEGVHRKEPQGAT

SEQ ID NO: 153 - protein, 316 aa, synthetic construct
  REGION 1..316: note = An artificially synthesized sequence
MTMETQMSQNVCPRNLWLLQPLTVLLLLASADSQAAPPKAVLKLEPPWINVLQEDSVTLTCQGARSPESDSIQWFHNGNLIPTHTQPSYRFKANNNDSGEYTCQTGQTSLSDPVHLTVLSEWLVLQTPHLEFQEGETIMLRCHSWKDKPLVKVTFFQNGKSQKFSHLDPTFSIPQANHSHSGDYHCTGNIGYTLFSSKPVTITVQVPSMGSSSPMGVIVAVVIATAVAAIVAAVVALIYCRKKRISANSTDPVKAAQFEPPGRQMIAIRKRQLEETNNDYETADGGYMTLNPRAPTDDDKNIYLTLPPNDHVNSNN

SEQ ID NO: 154 - protein, 291 aa, synthetic construct
  REGION 1..291: note = An artificially synthesized sequence
MGILSFLPVLATESDWADCKSPQPWGHMLLWTAVLFLAPVAGTPAAPPKAVLKLEPQWINVLQEDSVTLTCRGTHSPESDSIQWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAPSSSPMGIIVAVVTGIAVAAIVAAVVALIYCRKKRISANPTNPDEADKVGAENTITYSLLMHPDALEEPDDQNRI

SEQ ID NO: 155 - protein, 254 aa, synthetic construct [sequence omitted]

SEQ ID NO: 156 - protein, 233 aa, synthetic construct
  REGION 1..233: note = An artificially synthesized sequence
MWQLLLPTALLLLVSAGMRTEDLPKAVVFLEPQWYSVLEKDSVTLKCQGAYSPEDNSTQWFHNESLISSQASSYFIDAATVNDSGEYRCQTNLSTLSDPVQLEVHIGWLLLQAPRWVFKEEDPIHLRCHSWKNTALHKVTYLQNGKDRKYFHHNSDFHIPKATLKDSGSYFCRGLVGSKNVSSETVNITITQGLAVSTISSFSPPGYQVSFCLVMVLLFAVDTGLYFSVKTNI

SEQ ID NO: 157 - DNA, 1125 bp, synthetic construct
  misc_feature 1..1125: note = An artificially synthesized sequence
  source 1..1125: mol_type = other DNA
atgtggttcttgacaactctgctcctttgggttccagttgatgggcaagtggacaccacaaaggcagtgatcactttgcagcctccatgggtcagcgtgttccaagaggaaaccgtaaccttgcactgtgaggtgctccatctgcctgggagcagctctacacagtggtttctcaatggcacagccactcagacctcgacccccagctacagaatcacctctgccagtgtcaatgacagtggtgaatacaggtgccagagaggtctctcagggcgaagtgaccccatacagctggaaatccacagaggctggctactactgcaggtctccagcagagtcttcacggaaggagaacctctggccttgaggtgtcatgcgtggaaggataagctggtgtacaatgtgctttactatcgaaatggcaaagcctttaagtttttccactggaattctaacctcaccattctgaaaaccaacataagtcacaatggcacctaccattgctcaggcatgggaaagcatcgctacacatcagcaggaatatctgtcactgtgaaagagctatttccagctccagtgctgaatgcatctgtgacatccccactcctggaggggaatctggtcaccctgagctgtgaaacaaagttgctcttgcagaggcctggtttgcagctttacttctccttctacatgggcagcaagaccctgcgaggcaggaacacatcctctgaataccaaatactaactgctagaagagaagactctgggttatactggtgcgaggctgccacagaggatggaaatgtccttaagcgcagccctgagttggagcttcaagtgcttggcctccagttaccaactcctgtctggtttcatgtccttttctatctggcagtgggaataatgtttttagtgaacactgttctctgggtgacaatacgtaaagaactgaaaagaaagaaaaagtgggatttagaaatctctttggattctggtcatgagaagaaggtaatttccagccttcaagaagacagacatttagaagaagagctgaaatgtcaggaacaaaaagaagaacagctgcaggaaggggtgcaccggaaggagccccagggggccacgtag

SEQ ID NO: 158 - DNA, 951 bp, synthetic construct
  misc_feature 1..951: note = An artificially synthesized sequence
  source 1..951: mol_type = other DNA
atgactatggagacccaaatgtctcagaatgtatgtcccagaaacctgtggctgcttcaaccattgacagttttgctgctgctggcttctgcagacagtcaagctgctcccccaaaggctgtgctgaaacttgagcccccgtggatcaacgtgctccaggaggactctgtgactctgacatgccagggggctcgcagccctgagagcgactccattcagtggttccacaatgggaatctcattcccacccacacgcagcccagctacaggttcaaggccaacaacaatgacagcggggagtacacgtgccagactggccagaccagcctcagcgaccctgtgcatctgactgtgctttccgaatggctggtgctccagacccctcacctggagttccaggagggagaaaccatcatgctgaggtgccacagctggaaggacaagcctctggtcaaggtcacattcttccagaatggaaaatcccagaaattctcccatttggatcccaccttctccatcccacaagcaaaccacagtcacagtggtgattaccactgcacaggaaacataggctacacgctgttctcatccaagcctgtgaccatcactgtccaagtgcccagcatgggcagctcttcaccaatgggggtcattgtggctgtggtcattgcgactgctgtagcagccattgttgctgctgtagtggccttgatctactgcaggaaaaagcggatttcagccaattccactgatcctgtgaaggctgcccaatttgagccacctggacgtcaaatgattgccatcagaaagagacaacttgaagaaaccaacaatgactatgaaacagctgacggcggctacatgactctgaaccccagggcacctactgacgatgataaaaacatctacctgactcttcctcccaacgaccatgtcaacagtaataactaa

SEQ ID NO: 159 - DNA, 876 bp, synthetic construct
  misc_feature 1..876: note = An artificially synthesized sequence
  source 1..876: mol_type = other DNA
atgggaatcctgtcattcttacctgtccttgccactgagagtgactgggctgactgcaagtccccccagccttggggtcatatgcttctgtggacagctgtgctattcctggctcctgttgctgggacacctgcagctcccccaaaggctgtgctgaaactcgagccccagtggatcaacgtgctccaggaggactctgtgactctgacatgccgggggactcacagccctgagagcgactccattcagtggttccacaatgggaatctcattcccacccacacgcagcccagctacaggttcaaggccaacaacaatgacagcggggagtacacgtgccagactggccagaccagcctcagcgaccctgtgcatctgactgtgctttctgagtggctggtgctccagacccctcacctggagttccaggagggagaaaccatcgtgctgaggtgccacagctggaaggacaagcctctggtcaaggtcacattcttccagaatggaaaatccaagaaattttcccgttcggatcccaacttctccatcccacaagcaaaccacagtcacagtggtgattaccactgcacaggaaacataggctacacgctgtactcatccaagcctgtgaccatcactgtccaagctcccagctcttcaccgatggggatcattgtggctgtggtcactgggattgctgtagcggccattgttgctgctgtagtggccttgatctactgcaggaaaaagcggatttcagccaatcccactaatcctgatgaggctgacaaagttggggctgagaacacaatcacctattcacttctcatgcacccggatgctctggaagagcctgatgaccagaaccgtatttag

SEQ ID NO: 160 - DNA, 765 bp, synthetic construct
  misc_feature 1..765: note = An artificially synthesized sequence
  source 1..765: mol_type = other DNA
atgtggcagctgctcctcccaactgctctgctacttctagtttcagctggcatgcggactgaagatctcccaaaggctgtggtgttcctggagcctcaatggtacagggtgctcgagaaggacagtgtgactctgaagtgccagggagcctactcccctgaggacaattccacacagtggtttcacaatgagagcctcatctcaagccaggcctcgagctacttcattgacgctgccacagttgacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaaggcaggaagtattttcatcataattctgacttctacattccaaaagccacactcaaagacagcggctcctacttctgcagggggcttgttgggagtaaaaatgtgtcttcagagactgtgaacatcaccatcactcaaggtttgtcagtgtcaaccatctcatcattctttccacctgggtaccaagtctctttctgcttggtgatggtactcctttttgcagtggacacaggactatatttctctgtgaagacaaacattcgaagctcaacaagagactggaaggaccataaatttaaatggagaaaggaccctcaagacaaatga

SEQ ID NO: 161 - DNA, 660 bp, synthetic construct
  misc_feature 1..660: note = An artificially synthesized sequence
  source 1..660: mol_type = other DNA
atgtggcagctgctcctcccaactgctctgctacttctagtttcagctggcatgcggactgaagatctcccaaaggctgtggtgttcctggagcctcaatggtacagcgtgcttgagaaggacagtgtgactctgaagtgccagggagcctactcccctgaggacaattccacacagtggtttcacaatgagagcctcatctcaagccaggcctcgagctacttcattgacgctgccacagtcaacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaagacaggaagtattttcatcataattctgacttccacattccaaaagccacactcaaagatagcggctcctacttctgcagggggcttgttgggagtaaaaatgtgtcttcagagactgtgaacatcaccatcactcaaggtttggcagtgtcaaccatctcatcattctctccacctgggtaccaagtctctttctgcttggtgatggtactcctttttgca

SEQ ID NO: 162 - protein, 1221 aa, synthetic construct
  REGION 1..1221: note = An artificially synthesized sequence
MKLRLPDETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTSIISAVVGILLVVVLGVVFGILIKRRQQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLDHVRENRGRLGSQDLLNWCMQIAKGMSYLEDVRLVHRDLAARNVLVKSPNHVKITDFGLARLLDIDETEYHADGGKVPIKWMALESILRRRFTHQSDVWSYGVTVWELMTFGAKPYDGIPAREIPDLLEKGERLPQPPICTIDVYMIMVKCWMIDSECRPRFRELVSEFSRMARDPQRFVVIQNEDLGPDLDSTFYRSLLEDDDMGDLVDAEEYLVPQQGFFCPDPAPGAGGMVHHRHRSSSTRSGGGDLTLGLEPSEEEAPRSPLAPSEGAGSDVFDGDLGMGAAKGLQSLPTHDPSPLQRYSEDPTVPLPSETDGYVAPLTCSPQPEYVNQPDVRPQPPSPREGPLPAARPAGATLERPKTLSPGKNGVVKDVFAFGGAVENPEYLTPQGGAAPQPHPPPAFSPAFDNLYYWDQDPPERGAPPSTFKGTPTAENPEYLGLDVPV

SEQ ID NO: 163 - protein, 1236 aa, synthetic construct
  REGION 1..1236: note = An artificially synthesized sequence
MPRGSWKPQVCTGTDMKLRLPDETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTSIISAVVGILLVVVLGVVFGILIKRRQQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLDHVRENRGRLGSQDLLNWCMQIAKGMSYLEDVRLVHRDLAARNVLVKSPNHVKITDFGLARLLDIDETEYHADGGKVPIKWMALESILRRRFTHQSDVWSYGVTVWELMTFGAKPYDGIPAREIPDLLEKGERLPQPPICTIDVYMIMVKCWMIDSECRPRFRELVSEFSRMARDPQRFVVIQNEDLGPDLDSTFYRSLLEDDDMGDLVDAEEYLVPQQGFFCPDPAPGAGGMVHHRHRSSSTRSGGGDLTLGLEPSEEEAPRSPLAPSEGAGSDVFDGDLGMGAAKGLQSLPTHDPSPLQRYSEDPTVPLPSETDGYVAPLTCSPQPEYVNQPDVRPQPPSPREGPLPAARPAGATLERPKTLSPGKNGVVKDVFAFGGAVENPEYLTPQGGAAPQPHPPPAFSPAFDNLYYWDQDPPERGAPPSTFKGTPTAENPEYLGLDVPV

SEQ ID NO: 164 - protein, 1051 aa, synthetic construct
  REGION 1..1051: note = An artificially synthesized sequence
MELAALCRWGLLLALLPPGAASTQVCTGTDMKLRLPDETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTSIISAVVGILLVVVLGVVFGILIKRRQQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLDHVRENRGRLGSQDLLNWCMQIAKGMSYLEDVRLVHRDLAARNVLVKSPNHVKITDFGLARLLDIDETEYHADGGKVPIKWMALESILRRRFTHQSDVWSYGVTVWELMTFGAKPYDGIPAREIPDLLEKGERLPQPPICTIDVYMIMVKCWMIDSECRPRFRELVSEFSRMARDPQRFVVIQNEDLGPDLDSTFYRSLLEDDDMGDLVDAEEYLVPQQGFFCPDPAPGAGGMVHHRHRSSSTRNM

SEQ ID NO: 165 - protein, 601 aa, synthetic construct
  REGION 1..601: note = An artificially synthesized sequence
MKLRLPDETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHS

SEQ ID NO: 166 - protein, 1251 aa, synthetic construct
  REGION 1..1251: note = An artificially synthesized sequence
MELAALCRWGLLLALLPPGAASTQVCTGTDMKLRLPDETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTSIISAVVGILLVVVLGVVFGILIKRRQQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLDHVRENRGRLGSQDLLNWCMQIAKGMSYLEDVRLVHRDLAARNVLVKSPNHVKITDFGLARLLDIDETEYHADGGKVPIKWMALESILRRRFTHQSDVWSYGVTVWELMTFGAKPYDGIPAREIPDLLEKGERLPQPPICTIDVYMIMVKCWMIDSECRPRFRELVSEFSRMARDPQRFVVIQNEDLGPDLDSTFYRSLLEDDDMGDLVDAEEYLVPQQGFFCPDPAPGAGGMVHHRHRSSSTRSGGGDLTLGLEPSEEEAPRSPLAPSEGAGSDVFDGDLGMGAAKGLQSLPTHDPSPLQRYSEDPTVPLPSETDGYVAPLTCSPQPEYVNQPDVRPQPPSPREGPLPAARPAGATLERPKTLSPGKNGVVKDVFAFGGAVENPEYLTPQGGAAPQPHPPPAFSPAFDNLYYWDQDPPERGAPPSTFKGTPTAENPEYLGLDVPV

SEQ ID NO: 167 - protein, 4 aa, synthetic construct
  REGION 1..4: note = an artificially synthesized sequence
GGGS

SEQ ID NO: 168 - protein, 4 aa, synthetic construct
  REGION 1..4: note = an artificially synthesized sequence
SGGG

SEQ ID NO: 169 - protein, 5 aa, synthetic construct
  REGION 1..5: note = an artificially synthesized sequence
GGGGS

SEQ ID NO: 170 - protein, 5 aa, synthetic construct
  REGION 1..5: note = an artificially synthesized sequence
SGGGG

SEQ ID NO: 171 - protein, 6 aa, synthetic construct
  REGION 1..6: note = an artificially synthesized sequence
GGGGGS

SEQ ID NO: 172 - protein, 6 aa, synthetic construct
  REGION 1..6: note = an artificially synthesized sequence
SGGGGG

SEQ ID NO: 173 - protein, 7 aa, synthetic construct
  REGION 1..7: note = an artificially synthesized sequence
GGGGGGS

SEQ ID NO: 174 - protein, 7 aa, synthetic construct
  REGION 1..7: note = an artificially synthesized sequence
SGGGGGG

SEQ ID NO: 175 - protein, 19 aa, synthetic construct
  REGION 1..19: note = An artificially synthesized sequence
MGWSCIILFLVATATGVHS

(19) United States
(12) Patent Application Publication
(10) Pub. No.: US 2026/0274987 A1
(43) Pub. Date: Sep. 17, 2026

(54) HER2 S310F SPECIFIC ANTIGEN-BINDING MOLECULES

(71) Applicant: CHUGAI SEIYAKU KABUSHIKI KAISHA, Tokyo (JP)

(72) Inventors: Tatsushi KODAMA, Singapore (SG); Yang SUN, Singapore (SG)

(73) Assignee: CHUGAI SEIYAKU KABUSHIKI KAISHA, Tokyo (JP)

(21) Appl. No.: 19/690,435

(22) Filed: May 28, 2026

Related U.S. Application Data
(62) Division of application No. 17/290,439, filed on Nov. 5, 2019.

(30) Foreign Application Priority Data
Nov. 5, 2018 (JP) 2018-207927

(51) Int. Cl. C07K 16/32; C07K 16/28; G01N 33/575

(52) U.S. Cl. CPC C07K 16/32; C07K 16/2809; G01N 33/5759; C07K 2317/31; C07K 2317/55; C07K 2317/92; G01N 2333/82

(57) ABSTRACT

An objective of the present invention is to provide antigen-binding molecules that specifically bind to HER2 mutant. The present invention provides antigen-binding molecules that specifically bind to HER2 mutant, and are capable of modulating and/or activating an immune response; compositions comprising the antigen-binding molecule; and methods of using the same.

### CROSS-REFERENCE TO RELATED APPLICATIONS

This application is a divisional of U.S. patent application Ser. No. 17/290,439, filed Apr. 30, 2021, which is a U.S. National Phase of PCT Application No. PCT/JP2019/043179, filed Nov. 5, 2019, which claims the benefit of Japanese Patent Application No. 2018-207927, filed Nov. 5, 2018, each of which is incorporated herein by reference in its entirety.

### REFERENCE TO SEQUENCE LISTING SUBMITTED ELECTRONICALLY

The content of the electronically submitted sequence listing (Name: 6663_0403 Sequence_Listing.xml; Size: 240,481 bytes; and Date of Creation: May 26, 2026) filed with the application is incorporated herein by reference in its entirety.

### TECHNICAL FIELD

The present invention relates to antigen-binding molecules that specifically bind to HER2 (human epidermal growth factor receptor 2) with S310F mutation (hereinafter also referred to as HER2 S310F); and multispecific antigen-binding molecules that comprise a first domain comprising a first antibody variable region which specifically binds to HER2 S310F, and a second domain comprising a second antibody variable region which binds to T cell receptor complex, and a third domain comprising an Fc region; uses thereof.

### BACKGROUND ART

Cancer is one of the leading causes of death worldwide. With the exception of certain carcinomas, tumors are often difficult to cure effectively. Conventional cancer treatments include radiation therapy, chemotherapy, and immunotherapy. These treatments are often not effective enough and eventually cancer recurrence or metastasis occurs after the treatment. Lack of tumor specificity is one of the factors that limit the maximum efficacy; therefore, more tumor-specific molecular targeted therapy has become an additional viable option in cancer treatment.

Antibodies are drawing attention as pharmaceuticals since they are highly stable in plasma and have few side effects. Among multiple therapeutic antibodies, some types of antibodies require effector cells to exert an anti-tumor response. Antibody dependent cell-mediated cytotoxicity (ADCC) is a cytotoxicity exhibited by effector cells against antibody-bound cells via binding of the Fc region of the antibody to Fc receptors present on NK cells and macrophages. To date, multiple therapeutic antibodies that can induce ADCC to exert anti-tumor efficacy have been developed as pharmaceuticals for treating cancer (NPL 1). While therapies targeting tumor-specific expressed antigens using conventional therapeutic antibodies show excellent anti-tumor activities, administration of such antibodies does not always lead to satisfactory treatment outcomes.

In addition to the antibodies that adopt ADCC by recruiting NK cells or macrophages as effector cells, T cell-recruiting antibodies (TR antibodies) that adopt cytotoxicity by recruiting T cells as effector cells have been known since the 1980s (NPL 2 to 4). A TR antibody is a bispecific antibody that recognizes and binds to any one of the subunits forming a T-cell receptor complex on T-cells, in particular the CD3 epsilon chain, and an antigen on cancer cells. Several TR antibodies are currently being developed. Catumaxomab, which is a TR antibody against EpCAM, has been approved in the EU for the treatment of malignant ascites. Furthermore, a type of TR antibody called “bispecific T-cell engager (BiTE)” has been recently found to exhibit a strong anti-tumor activity (NPL 5 and 6). Blinatumomab, which is a BiTE molecule against CD19, received FDA approval first in 2014. Blinatumomab has been proved to exhibit a much stronger cytotoxic activity against CD19/CD20-positive cancer cells in vitro compared with Rituximab, which induces antibody-dependent cell-mediated cytotoxicity (ADCC) and complement-dependent cytotoxicity (CDC) (NPL 7).

However, it is known that a trifunctional antibody binds to both a T-cell and a cell such as an NK cell or macrophage at the same time in a cancer antigen-independent manner, and as a result receptors expressed on the cells are cross-linked, and expression of various cytokines is induced in a cancer antigen-independent manner. Systemic administration of a trifunctional antibody is thought to cause cytokine storm-like side effects as a result of such induction of cytokine expression. In fact, it has been reported that, in the phase I clinical trial, a very low dose of 5 micro g/body was the maximum tolerance dose for systemic administration of catumaxomab to patients with non-small cell lung cancer, and that administration of a higher dose causes various severe side effects (NPL 8). When administered at such a low dose, catumaxomab can never reach the effective blood level. That is, the expected anti-tumor effect cannot be achieved by administrating catumaxomab at such a low dose.

Meanwhile, unlike catumaxomab, BiTE has no Fc gamma receptor-binding site, and therefore it does not cross-link the receptors expressed on T-cells and cells such as NK cells and macrophages in a cancer antigen-independent manner. Thus, it has been demonstrated that BiTE does not cause cancer antigen-independent cytokine induction which is observed when catumaxomab is administered. However, since BiTE is a modified low-molecular-weight antibody molecule without an Fc region, the problem is that its blood half-life after administration to a patient is significantly shorter than IgG-type antibodies conventionally used as therapeutic antibodies. In fact, the blood half-life of BiTE administered in vivo has been reported to be about several hours (NPL 9 and 10). In the clinical trials of blinatumomab, it is administered by continuous intravenous infusion using a minipump. This administration method is not only extremely inconvenient for patients but also has the potential risk of medical accidents due to device malfunction or the like. Thus, it cannot be said that such an administration method is desirable.

HER2 (also known as ErbB2 or ErbB-2, human epidermal growth factor receptor 2, CD340) is a single-pass type 1 transmembrane protein and is a member of the epidermal growth factor receptor (EGFR) family of receptor tyrosine kinases which activates signaling pathways that regulates cellular proliferation and survival. HER2 overexpression and HER2 genetic alterations such as HER2 gene amplification, HER2 mutation (point mutation, insertion mutation, and deletion mutation) have been identified as oncogenic drivers and potential therapeutic targets in cancers (NPL 11 to 13). HER2-targeted therapies have dramatically improved the clinical outcomes of patients with breast cancer and gastric cancer that have HER2 overexpression or HER2 amplification. However, targeted therapy for cancer patients with HER2 mutation is still an unmet need in clinical setting.

HER2 mutations have been identified in various cancers. One example of HER2 mutation is HER2 S310F mutation which was observed in breast, non-small-cell-lung, bladder, colorectal, cervical, endometrial, and gastroesophageal cancers etc. (NPL 14).

To date, there is no report on antibodies that recognize HER2 S310F but do not recognize wild-type HER2. Therefore, the object of the present invention is to provide monoclonal antibodies which binds to HER2 S310F but do not bind to wild-type HER2.

### CITATION LIST

### Non Patent Literature

[NPL 1] Clin Cancer Res. 2010 Jan. 1; 16(1):11-20.
[NPL 2] Nature. 1985 Apr. 18-24; 314(6012):628-31.
[NPL 3] Int J Cancer. 1988 Apr. 15; 41(4):609-15.
[NPL 4] Proc Natl Acad Sci USA. 1986 March; 83(5):1453-7.
[NPL 5] Proc Natl Acad Sci USA. 1995 Jul. 18; 92(15):7021-5.
[NPL 6] Drug Discov Today. 2005 Sep. 15; 10(18):1237-44.
[NPL 7] Int J Cancer. 2002 Aug. 20; 100(6):690-7.
[NPL 8] Cancer Immunol Immunother (2007) 56 (10), 1637-44.
[NPL 9] Cancer Immunol Immunother. (2006) 55 (5), 503-14.
[NPL 10] Cancer Immunol Immunother. (2009) 58 (1), 95-109.
[NPL 11] Nat Rev Cancer. 2009 July; 9(7):463-75.
[NPL 12] Cancer Discov. 2013 February; 3(2):224-37.
[NPL 13] Cancer Discov. 2015 August; 5(8):832-41.
[NPL 14] Nature. 2018 Feb. 8; 554(7691):189-194.

### SUMMARY OF INVENTION

### Technical Problem

An objective of the present invention is to provide antigen-binding molecules that bind to a HER2 mutant, in particular HER2 S310F with high specificity, and multispecific antigen-binding molecules thereof that enable cancer treatment by recruiting T cells close to HER2 S310F-expressing cells and induce cytotoxicity of T cells against HER2 S310F-expressing cancer cells; methods for producing the antigen-binding molecules, and therapeutic agents comprising such an antigen-binding molecule as an active ingredient for inducing cellular cytotoxicity. Another objective of the present invention is to provide pharmaceutical compositions for use in treating or preventing various cancers, which comprise one of the above-mentioned antigen-binding molecules as an active ingredient, and therapeutic methods using the pharmaceutical compositions.

### Solution to Problem

The inventors of the present invention have successfully generated antigen-binding molecules that show high specificity and binding activity against HER2 S310F, with low or no cross-reactivity against a wild type HER2. To our best knowledge, to-date there has been no report on antibody that specifically recognizes a HER2 having single amino acid residue mutation (e.g. S310F) but does not recognize wild-type HER2. Such mutant specific anti-HER2 antigen-binding molecules could demonstrate superior specificity as they only target tumor cells expressing the tumor-specific antigen (i.e. HER2 S310F); whereas conventional anti-HER2 antibodies target/bind to wild type HER2 which is also expressed in normal tissues and hence could result in on-target toxicity and damaged healthy organs. Compared to conventional anti-HER2 antibodies, it is expected that the HER2 S310F mutant-specific antigen-binding molecules of the present invention (e.g. anti-HER2 S310F antibodies) are superior therapeutic agents in terms of high precision and tumor-targeting specificity, as well as its safety (reduced toxicity) due to the lack of HER2 S310F expression in nontumor tissues.

In another aspect, the inventors have generated multispecific antigen-binding molecules that comprise a first domain comprising a first antibody variable region which specifically binds to HER2 S310F, and a second domain comprising a second antibody variable region which binds to T-cell receptor complex (e.g. CD3), which can effectively kill cells expressing HER2 S310F, and exert a superior cytotoxic/antitumor activity. The multispecific antigen-binding molecules and pharmaceutical compositions can treat various cancers, especially those associated with HER2 S310F mutation such as HER2 S310F-positive tumors, which comprises the antigen-binding molecule as an active ingredient. Multispecific antigen-binding molecules of the present invention have strong anti-tumor activity, inducing cellular cytotoxicity, and can specifically target and damage HER2 S310F-expressing cells (but not HER2-expressing cells), thus enable high precise and specific treatment and prevention of HER2 S310F-positive cancers. Furthermore, the multispecific antigen-binding molecules of the present invention have a long half-life in blood, as well as excellent safety properties that result in no induction of cancer antigen-independent cytokine storm or such. This allows desirable treatments that are highly safe and convenient, and reduces the physical burden for patients.

More specifically, the present invention provides the following:
[1] An antigen-binding molecule comprising a first antigen-binding domain which specifically binds to a human HER2 with S310F mutation (HER2 S310F) and has weaker or no binding activity to a wild type human HER2.
[2] An antigen-binding molecule or the antigen-binding molecule of [1], which specifically binds an epitope comprising a Phenylalanine residue at position 310 of human HER2.
[3] An antigen-binding molecule or the antigen-binding molecule of any one of [1]-[2], which binds to human HER2 S310F with a KD of less than 100 nanomolar (nM), preferably as measured by surface plasmon resonance (SPR) at the following condition: 37 degrees Celcius, pH 7.4, 20 mM ACES, 150 mM NaCl, 0.05% Tween 20, 0.005% NaN3; the antigen-binding molecule is immobilized on a CM4 sensor chip, the HER2 S310F antigen serves as analyte.
[4] An antigen-binding molecule or the antigen-binding molecule of any one of [1]-[3], which has at least 10-fold, 100-fold, 1,000-fold or 10,000-fold, or 100,000-fold smaller KD ratio of the KD value for binding to human HER2 S310F to the KD value for binding to human wild type HER2 (i.e. KD_{HER2 S310F}/KD_{wild type HER2}), preferably as measured by SPR at the following condition: 37 degrees Celcius, pH 7.4, 20 mM ACES, 150 mM NaCl, 0.05% Tween 20, 0.005% NaN3; the antigen-binding molecule is immobilized on a CM4 sensor chip, the antigen serves as analyte.
[5] The antigen-binding molecule of any one of [1]-[4], which does not bind to wild type human HER2.
[6] An antigen-binding molecule or the antigen-binding molecule of any one of [1]-[5], which prevents ligand-independent dimerization of human HER2 S310F.
[6A] The antigen-binding molecule of any one of [1]-[5], which is a monoclonal antibody.
[6B] The antigen-binding molecule of any one of [1]-[5], which is a human, humanized, or chimeric antibody.
[6C] The antigen-binding molecule of any one of [1]-[5], which is a human IgG1.
[7] The antigen-binding molecule of any one of [1]-[6], further comprises a second antigen-binding domain that specifically binds to a T-cell receptor complex molecule.
[8] The antigen-binding molecule of [7], wherein the T-cell receptor complex molecule is CD3, preferably a human CD3 epsilon chain.
[9] The antigen-binding molecule of any one of [7]-[8], which is a multispecific antigen-binding molecule, preferably a bispecific antibody.
[9A] The antigen-binding molecule of any one of [1]-[9], further comprises a Fc domain.
[10] The antigen-binding molecule of [9A], wherein the Fc domain is a Fc domain that has a reduced Fc gamma receptor-binding activity compared to a wild type Fc domain of IgG1, preferably human IgG1.
[10A] The antigen-binding molecule of [10], wherein the Fc region is an Fc region with mutation of at least one amino acid selected from the following amino acid positions specified by EU numbering: position 220, position 226, position 229, position 231, position 232, position 233, position 234, position 235, position 236, position 237, position 238, position 239, position 240, position 264, position 265, position 266, position 267, position 269, position 270, position 295, position 296, position 297, position 298, position 299, position 300, position 325, position 327, position 328, position 329, position 330, position 331, and position 332.
[11] The antigen-binding molecule of any one of [1]-[10A], which has a cytotoxicity activity.
[12] The antigen-binding molecule of [11], which comprises a second antigen-binding domain that specifically binds to a T-cell receptor complex molecule, wherein the cytotoxic activity is T-cell-dependent cytotoxic activity (TDCC).
[13] The antigen-binding molecule of any one of [1]-[12], wherein the first antigen-binding domain and/or the second antigen-binding domain comprises a Fab, Fv, scFv or VHH structure.
[14] The antigen-binding molecule of any one of [1]-[13], wherein the first antigen-binding domain and/or the second antigen-binding domain comprises a Fab structure.
[15] The antigen-binding molecule of any one of [1]-[14], which comprises a F(ab′)2 structure.
[16] The antigen-binding molecule of any one of [1]-[15], wherein the first antigen-binding domain comprises any one of the following antibody variable regions:
(a1) an antibody variable region that comprises a heavy chain complementarity determining region 1 (HCDR1) comprising an amino acid sequence that is at least 95% identical to SEQ ID NO: 29, a heavy chain complementarity determining region 2 (HCDR2) comprising an amino acid sequence that is at least 95% identical to SEQ ID NO: 41, a heavy chain complementarity determining region 3 (HCDR3) comprising an amino acid sequence that is at least 95% identical to SEQ ID NO: 53, a light chain complementarity determining region 1 (LCDR1) comprising an amino acid sequence that is at least 95% identical to SEQ ID NO: 65, a light chain complementarity determining region 2 (LCDR2) comprising an amino acid sequence that is at least 95% identical to SEQ ID NO: 77, and a light chain complementarity determining region 3 (LCDR3) comprising an amino acid sequence that is at least 95% identical to SEQ ID NO: 89;
(a2) an antibody variable region that comprises a heavy chain complementarity determining region 1 (HCDR1) comprising an amino acid sequence that is at least 95% identical to SEQ ID NO: 30, a heavy chain complementarity determining region 2 (HCDR2) comprising an amino acid sequence that is at least 95% identical to SEQ ID NO: 42, a heavy chain complementarity determining region 3 (HCDR3) comprising an amino acid sequence that is at least 95% identical to SEQ ID NO: 54, a light chain complementarity determining region 1 (LCDR1) comprising an amino acid sequence that is at least 95% identical to SEQ ID NO: 66, a light chain complementarity determining region 2 (LCDR2) comprising an amino acid sequence that is at least 95% identical to SEQ ID NO: 78, and a light chain complementarity determining region 3 (LCDR3) comprising an amino acid sequence that is at least 95% identical to SEQ ID NO: 90;
(a3) an antibody variable region that comprises a heavy chain complementarity determining region 1 (HCDR1) comprising an amino acid sequence that is at least 95% identical to SEQ ID NO: 31, a heavy chain complementarity determining region 2 (HCDR2) comprising an amino acid sequence that is at least 95% identical to SEQ ID NO: 43, a heavy chain complementarity determining region 3 (HCDR3) comprising an amino acid sequence that is at least 95% identical to SEQ ID NO: 55, a light chain complementarity determining region 1 (LCDR1) comprising an amino acid sequence that is at least 95% identical to SEQ ID NO: 67, a light chain complementarity determining region 2 (LCDR2) comprising an amino acid sequence that is at least 95% identical to SEQ ID NO: 79, and a light chain complementarity determining region 3 (LCDR3) comprising an amino acid sequence that is at least 95% identical to SEQ ID NO: 91;
(a4) an antibody variable region that comprises a heavy chain complementarity determining region 1 (HCDR1) comprising an amino acid sequence that is at least 95% identical to SEQ ID NO: 32, a heavy chain complementarity determining region 2 (HCDR2) comprising an amino acid sequence that is at least 95% identical to SEQ ID NO: 44, a heavy chain complementarity determining region 3 (HCDR3) comprising an amino acid sequence that is at least 95% identical to SEQ ID NO: 56, a light chain complementarity determining region 1 (LCDR1) comprising an amino acid sequence that is at least 95% identical to SEQ ID NO: 68, a light chain complementarity determining region 2 (LCDR2) comprising an amino acid sequence that is at least 95% identical to SEQ ID NO: 80, and a light chain complementarity determining region 3 (LCDR3) comprising an amino acid sequence that is at least 95% identical to SEQ ID NO: 92;
(a5) an antibody variable region that comprises a heavy chain complementarity determining region 1 (HCDR1) comprising an amino acid sequence that is at least 95% identical to SEQ ID NO: 33, a heavy chain complementarity determining region 2 (HCDR2) comprising an amino acid sequence that is at least 95% identical to SEQ ID NO: 45, a heavy chain complementarity determining region 3 (HCDR3) comprising an amino acid sequence that is at least 95% identical to SEQ ID NO: 57, a light chain complementarity determining region 1 (LCDR1) comprising an amino acid sequence that is at least 95% identical to SEQ ID NO: 69, a light chain complementarity determining region 2 (LCDR2) comprising an amino acid sequence that is at least 95% identical to SEQ ID NO: 81, and a light chain complementarity determining region 3 (LCDR3) comprising an amino acid sequence that is at least 95% identical to SEQ ID NO: 93;
(a6) an antibody variable region that comprises a heavy chain complementarity determining region 1 (HCDR1) comprising an amino acid sequence that is at least 95% identical to SEQ ID NO: 34, a heavy chain complementarity determining region 2 (HCDR2) comprising an amino acid sequence that is at least 95% identical to SEQ ID NO: 46, a heavy chain complementarity determining region 3 (HCDR3) comprising an amino acid sequence that is at least 95% identical to SEQ ID NO: 58, a light chain complementarity determining region 1 (LCDR1) comprising an amino acid sequence that is at least 95% identical to SEQ ID NO: 70, a light chain complementarity determining region 2 (LCDR2) comprising an amino acid sequence that is at least 95% identical to SEQ ID NO: 82, and a light chain complementarity determining region 3 (LCDR3) comprising an amino acid sequence that is at least 95% identical to SEQ ID NO: 94;
(a7) an antibody variable region that comprises a heavy chain complementarity determining region 1 (HCDR1) comprising an amino acid sequence that is at least 95% identical to SEQ ID NO: 35, a heavy chain complementarity determining region 2 (HCDR2) comprising an amino acid sequence that is at least 95% identical to SEQ ID NO: 47, a heavy chain complementarity determining region 3 (HCDR3) comprising an amino acid sequence that is at least 95% identical to SEQ ID NO: 59, a light chain complementarity determining region 1 (LCDR1) comprising an amino acid sequence that is at least 95% identical to SEQ ID NO: 71, a light chain complementarity determining region 2 (LCDR2) comprising an amino acid sequence that is at least 95% identical to SEQ ID NO: 83, and a light chain complementarity determining region 3 (LCDR3) comprising an amino acid sequence that is at least 95% identical to SEQ ID NO: 95;
(a8) an antibody variable region that comprises a heavy chain complementarity determining region 1 (HCDR1) comprising an amino acid sequence that is at least 95% identical to SEQ ID NO: 36, a heavy chain complementarity determining region 2 (HCDR2) comprising an amino acid sequence that is at least 95% identical to SEQ ID NO: 48, a heavy chain complementarity determining region 3 (HCDR3) comprising an amino acid sequence that is at least 95% identical to SEQ ID NO: 60, a light chain complementarity determining region 1 (LCDR1) comprising an amino acid sequence that is at least 95% identical to SEQ ID NO: 72, a light chain complementarity determining region 2 (LCDR2) comprising an amino acid sequence that is at least 95% identical to SEQ ID NO: 84, and a light chain complementarity determining region 3 (LCDR3) comprising an amino acid sequence that is at least 95% identical to SEQ ID NO: 96;
(a9) an antibody variable region that comprises a heavy chain complementarity determining region 1 (HCDR1) comprising an amino acid sequence that is at least 95% identical to SEQ ID NO: 37, a heavy chain complementarity determining region 2 (HCDR2) comprising an amino acid sequence that is at least 95% identical to SEQ ID NO: 49, a heavy chain complementarity determining region 3 (HCDR3) comprising an amino acid sequence that is at least 95% identical to SEQ ID NO: 61, a light chain complementarity determining region 1 (LCDR1) comprising an amino acid sequence that is at least 95% identical to SEQ ID NO: 73, a light chain complementarity determining region 2 (LCDR2) comprising an amino acid sequence that is at least 95% identical to SEQ ID NO: 85, and a light chain complementarity determining region 3 (LCDR3) comprising an amino acid sequence that is at least 95% identical to SEQ ID NO: 97;
(a10) an antibody variable region that comprises a heavy chain complementarity determining region 1 (HCDR1) comprising an amino acid sequence that is at least 95% identical to SEQ ID NO: 38, a heavy chain complementarity determining region 2 (HCDR2) comprising an amino acid sequence that is at least 95% identical to SEQ ID NO: 50, a heavy chain complementarity determining region 3 (HCDR3) comprising an amino acid sequence that is at least 95% identical to SEQ ID NO: 62, a light chain complementarity determining region 1 (LCDR1) comprising an amino acid sequence that is at least 95% identical to SEQ ID NO: 74, a light chain complementarity determining region 2 (LCDR2) comprising an amino acid sequence that is at least 95% identical to SEQ ID NO: 86, and a light chain complementarity determining region 3 (LCDR3) comprising an amino acid sequence that is at least 95% identical to SEQ ID NO: 98;
(a11) an antibody variable region that comprises a heavy chain complementarity determining region 1 (HCDR1) comprising an amino acid sequence that is at least 95% identical to SEQ ID NO: 39, a heavy chain complementarity determining region 2 (HCDR2) comprising an amino acid sequence that is at least 95% identical to SEQ ID NO: 51, a heavy chain complementarity determining region 3 (HCDR3) comprising an amino acid sequence that is at least 95% identical to SEQ ID NO: 63, a light chain complementarity determining region 1 (LCDR1) comprising an amino acid sequence that is at least 95% identical to SEQ ID NO: 75, a light chain complementarity determining region 2 (LCDR2) comprising an amino acid sequence that is at least 95% identical to SEQ ID NO: 87, and a light chain complementarity determining region 3 (LCDR3) comprising an amino acid sequence that is at least 95% identical to SEQ ID NO: 99;
(a12) an antibody variable region that comprises a heavy chain complementarity determining region 1 (HCDR1) comprising an amino acid sequence that is at least 95% identical to SEQ ID NO: 40, a heavy chain complementarity determining region 2 (HCDR2) comprising an amino acid sequence that is at least 95% identical to SEQ ID NO: 52, a heavy chain complementarity determining region 3 (HCDR3) comprising an amino acid sequence that is at least 95% identical to SEQ ID NO: 64, a light chain complementarity determining region 1 (LCDR1) comprising an amino acid sequence that is at least 95% identical to SEQ ID NO: 76, a light chain complementarity determining region 2 (LCDR2) comprising an amino acid sequence that is at least 95% identical to SEQ ID NO: 88, and a light chain complementarity determining region 3 (LCDR3) comprising an amino acid sequence that is at least 95% identical to SEQ ID NO: 100;
(b1) an antibody variable region that comprises a HCDR1 comprising an amino acid sequence of SEQ ID NO: 29, a HCDR2 comprising an amino acid sequence of SEQ ID NO: 41, a HCDR3 comprising an amino acid sequence of SEQ ID NO: 53, a LCDR1 comprising an amino acid sequence of SEQ ID NO: 65, a LCDR2 comprising an amino acid sequence of SEQ ID NO: 77, and a LCDR3 comprising an amino acid sequence of SEQ ID NO: 89;
(b2) an antibody variable region that comprises a HCDR1 comprising an amino acid sequence of SEQ ID NO: 30, a HCDR2 comprising an amino acid sequence of SEQ ID NO: 42, a HCDR3 comprising an amino acid sequence of SEQ ID NO: 54, a LCDR1 comprising an amino acid sequence of SEQ ID NO: 66, a LCDR2 comprising an amino acid sequence of SEQ ID NO: 78, and a LCDR3 comprising an amino acid sequence of SEQ ID NO: 90;
(b3) an antibody variable region that comprises a HCDR1 comprising an amino acid sequence of SEQ ID NO: 31, a HCDR2 comprising an amino acid sequence of SEQ ID NO: 43, a HCDR3 comprising an amino acid sequence of SEQ ID NO: 55, a LCDR1 comprising an amino acid sequence of SEQ ID NO: 67, a LCDR2 comprising an amino acid sequence of SEQ ID NO: 79, and a LCDR3 comprising an amino acid sequence of SEQ ID NO: 91;
(b4) an antibody variable region that comprises a HCDR1 comprising an amino acid sequence of SEQ ID NO: 32, a HCDR2 comprising an amino acid sequence of SEQ ID NO: 44, a HCDR3 comprising an amino acid sequence of SEQ ID NO: 56, a LCDR1 comprising an amino acid sequence of SEQ ID NO: 68, a LCDR2 comprising an amino acid sequence of SEQ ID NO: 80, and a LCDR3 comprising an amino acid sequence of SEQ ID NO: 92;
(b5) an antibody variable region that comprises a HCDR1 comprising an amino acid sequence of SEQ ID NO: 33, a HCDR2 comprising an amino acid sequence of SEQ ID NO: 45, a HCDR3 comprising an amino acid sequence of SEQ ID NO: 57, a LCDR1 comprising an amino acid sequence of SEQ ID NO: 69, a LCDR2 comprising an amino acid sequence of SEQ ID NO: 81, and a LCDR3 comprising an amino acid sequence of SEQ ID NO: 93;
(b6) an antibody variable region that comprises a HCDR1 comprising an amino acid sequence of SEQ ID NO: 34, a HCDR2 comprising an amino acid sequence of SEQ ID NO: 46, a HCDR3 comprising an amino acid sequence of SEQ ID NO: 58, a LCDR1 comprising an amino acid sequence of SEQ ID NO: 70, a LCDR2 comprising an amino acid sequence of SEQ ID NO: 82, and a LCDR3 comprising an amino acid sequence of SEQ ID NO: 94;
(b7) an antibody variable region that comprises a HCDR1 comprising an amino acid sequence of SEQ ID NO: 35, a HCDR2 comprising an amino acid sequence of SEQ ID NO: 47, a HCDR3 comprising an amino acid sequence of SEQ ID NO: 59, a LCDR1 comprising an amino acid sequence of SEQ ID NO: 71, a LCDR2 comprising an amino acid sequence of SEQ ID NO: 83, and a LCDR3 comprising an amino acid sequence of SEQ ID NO: 95;
(b8) an antibody variable region that comprises a HCDR1 comprising an amino acid sequence of SEQ ID NO: 36, a HCDR2 comprising an amino acid sequence of SEQ ID NO: 48, a HCDR3 comprising an amino acid sequence of SEQ ID NO: 60, a LCDR1 comprising an amino acid sequence of SEQ ID NO: 72, a LCDR2 comprising an amino acid sequence of SEQ ID NO: 84, and a LCDR3 comprising an amino acid sequence of SEQ ID NO: 96;
(b9) an antibody variable region that comprises a HCDR1 comprising an amino acid sequence of SEQ ID NO: 37, a HCDR2 comprising an amino acid sequence of SEQ ID NO: 49, a HCDR3 comprising an amino acid sequence of SEQ ID NO: 61, a LCDR1 comprising an amino acid sequence of SEQ ID NO: 73, a LCDR2 comprising an amino acid sequence of SEQ ID NO: 85, and a LCDR3 comprising an amino acid sequence of SEQ ID NO: 97;
(b10) an antibody variable region that comprises a HCDR1 comprising an amino acid sequence of SEQ ID NO: 38, a HCDR2 comprising an amino acid sequence of SEQ ID NO: 50, a HCDR3 comprising an amino acid sequence of SEQ ID NO: 62, a LCDR1 comprising an amino acid sequence of SEQ ID NO: 74, a LCDR2 comprising an amino acid sequence of SEQ ID NO: 86, and a LCDR3 comprising an amino acid sequence of SEQ ID NO: 98;
(b11) an antibody variable region that comprises a HCDR1 comprising an amino acid sequence of SEQ ID NO: 39, a HCDR2 comprising an amino acid sequence of SEQ ID NO: 51, a HCDR3 comprising an amino acid sequence of SEQ ID NO: 63, a LCDR1 comprising an amino acid sequence of SEQ ID NO: 75, a LCDR2 comprising an amino acid sequence of SEQ ID NO: 87, and a LCDR3 comprising an amino acid sequence of SEQ ID NO: 99;
(b12) an antibody variable region that comprises a HCDR1 comprising an amino acid sequence of SEQ ID NO: 40, a HCDR2 comprising an amino acid sequence of SEQ ID NO: 52, a HCDR3 comprising an amino acid sequence of SEQ ID NO: 64, a LCDR1 comprising an amino acid sequence of SEQ ID NO: 76, a LCDR2 comprising an amino acid sequence of SEQ ID NO: 88, and a LCDR3 comprising an amino acid sequence of SEQ ID NO: 100;
(c1) an antibody variable region that comprises a heavy chain variable domain (VH) of SEQ ID NO: 5, and a light chain variable domain (VL) of SEQ ID NO: 17;
(c2) an antibody variable region that comprises a heavy chain variable domain (VH) of SEQ ID NO: 6, and a light chain variable domain (VL) of SEQ ID NO: 18;
(c3) an antibody variable region that comprises a heavy chain variable domain (VH) of SEQ ID NO: 7, and a light chain variable domain (VL) of SEQ ID NO: 19;
(c4) an antibody variable region that comprises a heavy chain variable domain (VH) of SEQ ID NO: 8, and a light chain variable domain (VL) of SEQ ID NO: 20;
(c5) an antibody variable region that comprises a heavy chain variable domain (VH) of SEQ ID NO: 9, and a light chain variable domain (VL) of SEQ ID NO: 21;
(c6) an antibody variable region that comprises a heavy chain variable domain (VH) of SEQ ID NO: 10, and a light chain variable domain (VL) of SEQ ID NO: 22;
(c7) an antibody variable region that comprises a heavy chain variable domain (VH) of SEQ ID NO: 11, and a light chain variable domain (VL) of SEQ ID NO: 23;
(c8) an antibody variable region that comprises a heavy chain variable domain (VH) of SEQ ID NO: 12, and a light chain variable domain (VL) of SEQ ID NO: 24;
(c9) an antibody variable region that comprises a heavy chain variable domain (VH) of SEQ ID NO: 13, and a light chain variable domain (VL) of SEQ ID NO: 25;
(c10) an antibody variable region that comprises a heavy chain variable domain (VH) of SEQ ID NO: 14, and a light chain variable domain (VL) of SEQ ID NO: 26;
(c11) an antibody variable region that comprises a heavy chain variable domain (VH) of SEQ ID NO: 15, and a light chain variable domain (VL) of SEQ ID NO: 27;
(c12) an antibody variable region that comprises a heavy chain variable domain (VH) of SEQ ID NO: 16, and a light chain variable domain (VL) of SEQ ID NO: 28;
(d) an antibody variable region that competes for binding to HER2 with any one of the antibody variable regions of (a1) to (c12);
(e) an antibody variable region that binds to the same epitope on HER2 with any one of the antibody variable regions of (a1) to (c12).
[17] The antigen-binding molecule of any one of [1]-[16], wherein the second antigen-binding domain comprises any one of the following antibody variable regions:
(a) an antibody variable region that comprises a HCDR1 comprising an amino acid sequence of SEQ ID NO: 103, a HCDR2 comprising an amino acid sequence of SEQ ID NO: 104, a HCDR3 comprising an amino acid sequence of SEQ ID NO: 105, a LCDR1 comprising an amino acid sequence of SEQ ID NO: 106, a LCDR2 comprising an amino acid sequence of SEQ ID NO: 107, and a LCDR3 comprising an amino acid sequence of SEQ ID NO: 108;
(b) an antibody variable region that comprises a heavy chain variable domain (VH) of SEQ ID NO: 101, and a light chain variable domain (VL) of SEQ ID NO: 102.
[18] A pharmaceutical composition for use in inducing cellular cytotoxicity that comprises any of the antigen-binding molecules of [1]-[17] and a pharmaceutically acceptable excipient.
[19] A pharmaceutical composition for use in treating cancer that comprises any of the antigen-binding molecules of [1]-[17] and a pharmaceutically acceptable excipient.
[20] A diagnostic kit comprising the antigen-binding molecule of any one of [1] to [17].
[21] An immunoconjugate comprising the antigen-binding molecule of any one of [1] to [17] and a cytotoxic agent.
[22] The antigen-binding molecule of any one of [1] to [17] for use as a medicament.
[23] The antigen-binding molecule of any one of [1] to [17] for use in treating, preventing or diagnosing cancer.
[24] Use of the antigen-binding molecule of any one of [1] to [17] in the manufacture of a medicament for treatment of cancer.
[25] A method of diagnosing cancer in an individual, or of predicting cancer treatment response in an individual, comprising determining the presence of human HER2 S310F using the antigen-binding molecule of any one of [1] to [17].
[26] A method of detecting the presence of human HER2 S310F in an isolated sample, comprising incubating the sample with the antigen-binding molecule of any one of [1] to [17] and detecting the presence of a complex formed between the antigen-binding molecule and human HER2 S310F.
[27] The pharmaceutical composition of any of [18]-[19], the diagnostic kit of [20], the antigen-binding molecule for use of any of [22]-[23], the use of [24], the method of [25], wherein the cancer is characterized by expression of human HER2 S310F.
[28] The pharmaceutical composition of any of [18]-[19], the diagnostic kit of [20], the antigen-binding molecule for use of any of [22]-[23], the use of [24], the method of [25], wherein the cancer is bladder cancer, cervical cancer, colorectal cancer (CRC), non-small-cell lung cancer (NSCLC), esophagus cancer, head and neck cancer, skin cancer (melanoma), bile duct cancer, kidney cancer, stomach cancer, small intestine cancer, liver cancer, uterus cancer, duodenum cancer, breast cancer, gall bladder cancer, ovary cancer.
[29] A nucleic acid encoding the antigen-binding molecule of any one of [1]-[17], optionally in a vector.
[30] A cell comprising the nucleic acid of [29].
[31] A method of preparing the antigen-binding molecule of any one of [1]-[17], the method comprising expressing a nucleic acid encoding the antigen-binding molecule in a cell.

In one embodiment, the antigen-binding molecule of the present invention comprises a first antigen-binding domain which specifically binds to a human HER2 with S310F mutation (HER2 S310F) and has weaker or no binding activity to a wild type human HER2. In another embodiment, the antigen-binding molecule of the present invention comprises a first antigen-binding domain which specifically binds an epitope comprising a Phenylalanine residue at position 310 of human HER2. In yet another embodiment, the antigen-binding molecule of the present invention comprises a first antigen-binding domain which binds to human HER2 S310F with a KD of less than 100 nanomolar (nM), preferably as measured by surface plasmon resonance (SPR) at the following condition: 37 degrees Celcius (C), pH 7.4, 20 mM ACES, 150 mM NaCl, 0.05% Tween 20, 0.005% NaN3; the antigen-binding molecule is immobilized on a CM4 sensor chip, the HER2 S310F antigen serves as analyte. In an embodiment, the antigen-binding molecule of the present invention, which is specific to the HER2 S310F mutant, comprises a first antigen-binding domain which has a lower KD value for binding to human HER2 S310F (KD_{HER2 S310F}) compared to the KD value for binding to human wild type HER2 (KD_{wild type HER2}). In a preferred embodiment, the antigen-binding molecule of the present invention comprises a first antigen-binding domain which has at least 10-fold, 100-fold, 1,000-fold or 10,000-fold, or 100,000-fold lower (i.e., 1/10, 1/100, 1/1000, 1/10000, or 1/100000) KD value for binding to human HER2 S310F compared to the KD value for binding to human wild type HER2, preferably as measured by SPR at the following condition: 37 degrees C., pH 7.4, 20 mM ACES, 150 mM NaCl, 0.05% Tween 20, 0.005% NaN3; the antigen-binding molecule is immobilized on a CM4 sensor chip, the antigen serves as analyte. In a preferred embodiment, the antigen-binding molecule of the present invention comprises a first antigen-binding domain which specifically binds to a human HER2 with S310F mutation (HER2 S310F) but does not bind to wild type human HER2. In another preferred embodiment, the antigen-binding molecule of the present invention comprises a first antigen-binding domain which prevents ligand-independent dimerization of human HER2 S310F.

In one preferred embodiment, the antigen-binding molecule of the present invention comprises:
[A] a first antigen-binding domain comprising any one of the following antibody variable regions:
   (a1) an antibody variable region that comprises a heavy chain complementarity determining region 1 (HCDR1) comprising an amino acid sequence that is at least 95% identical to SEQ ID NO: 29, a heavy chain complementarity determining region 2 (HCDR2) comprising an amino acid sequence that is at least 95% identical to SEQ ID NO: 41, a heavy chain complementarity determining region 3 (HCDR3) comprising an amino acid sequence that is at least 95% identical to SEQ ID NO: 53, a light chain complementarity determining region 1 (LCDR1) comprising an amino acid sequence that is at least 95% identical to SEQ ID NO: 65, a light chain complementarity determining region 2 (LCDR2) comprising an amino acid sequence that is at least 95% identical to SEQ ID NO: 77, and a light chain complementarity determining region 3 (LCDR3) comprising an amino acid sequence that is at least 95% identical to SEQ ID NO: 89;
   (a2) an antibody variable region that comprises a heavy chain complementarity determining region 1 (HCDR1) comprising an amino acid sequence that is at least 95% identical to SEQ ID NO: 30, a heavy chain complementarity determining region 2 (HCDR2) comprising an amino acid sequence that is at least 95% identical to SEQ ID NO: 42, a heavy chain complementarity determining region 3 (HCDR3) comprising an amino acid sequence that is at least 95% identical to SEQ ID NO: 54, a light chain complementarity determining region 1 (LCDR1) comprising an amino acid sequence that is at least 95% identical to SEQ ID NO: 66, a light chain complementarity determining region 2 (LCDR2) comprising an amino acid sequence that is at least 95% identical to SEQ ID NO: 78, and a light chain complementarity determining region 3 (LCDR3) comprising an amino acid sequence that is at least 95% identical to SEQ ID NO: 90;
   (a3) an antibody variable region that comprises a heavy chain complementarity determining region 1 (HCDR1) comprising an amino acid sequence that is at least 95% identical to SEQ ID NO: 31, a heavy chain complementarity determining region 2 (HCDR2) comprising an amino acid sequence that is at least 95% identical to SEQ ID NO: 43, a heavy chain complementarity determining region 3 (HCDR3) comprising an amino acid sequence that is at least 95% identical to SEQ ID NO: 55, a light chain complementarity determining region 1 (LCDR1) comprising an amino acid sequence that is at least 95% identical to SEQ ID NO: 67, a light chain complementarity determining region 2 (LCDR2) comprising an amino acid sequence that is at least 95% identical to SEQ ID NO: 79, and a light chain complementarity determining region 3 (LCDR3) comprising an amino acid sequence that is at least 95% identical to SEQ ID NO: 91;
   (a4) an antibody variable region that comprises a heavy chain complementarity determining region 1 (HCDR1) comprising an amino acid sequence that is at least 95% identical to SEQ ID NO: 32, a heavy chain complementarity determining region 2 (HCDR2) comprising an amino acid sequence that is at least 95% identical to SEQ ID NO: 44, a heavy chain complementarity determining region 3 (HCDR3) comprising an amino acid sequence that is at least 95% identical to SEQ ID NO: 56, a light chain complementarity determining region 1 (LCDR1) comprising an amino acid sequence that is at least 95% identical to SEQ ID NO: 68, a light chain complementarity determining region 2 (LCDR2) comprising an amino acid sequence that is at least 95% identical to SEQ ID NO: 80, and a light chain complementarity determining region 3 (LCDR3) comprising an amino acid sequence that is at least 95% identical to SEQ ID NO: 92;
   (a5) an antibody variable region that comprises a heavy chain complementarity determining region 1 (HCDR1) comprising an amino acid sequence that is at least 95% identical to SEQ ID NO: 33, a heavy chain complementarity determining region 2 (HCDR2) comprising an amino acid sequence that is at least 95% identical to SEQ ID NO: 45, a heavy chain complementarity determining region 3 (HCDR3) comprising an amino acid sequence that is at least 95% identical to SEQ ID NO: 57, a light chain complementarity determining region 1 (LCDR1) comprising an amino acid sequence that is at least 95% identical to SEQ ID NO: 69, a light chain complementarity determining region 2 (LCDR2) comprising an amino acid sequence that is at least 95% identical to SEQ ID NO: 81, and a light chain complementarity determining region 3 (LCDR3) comprising an amino acid sequence that is at least 95% identical to SEQ ID NO: 93;
   (a6) an antibody variable region that comprises a heavy chain complementarity determining region 1 (HCDR1) comprising an amino acid sequence that is at least 95% identical to SEQ ID NO: 34, a heavy chain complementarity determining region 2 (HCDR2) comprising an amino acid sequence that is at least 95% identical to SEQ ID NO: 46, a heavy chain complementarity determining region 3 (HCDR3) comprising an amino acid sequence that is at least 95% identical to SEQ ID NO: 58, a light chain complementarity determining region 1 (LCDR1) comprising an amino acid sequence that is at least 95% identical to SEQ ID NO: 70, a light chain complementarity determining region 2 (LCDR2) comprising an amino acid sequence that is at least 95% identical to SEQ ID NO: 82, and a light chain complementarity determining region 3 (LCDR3) comprising an amino acid sequence that is at least 95% identical to SEQ ID NO: 94;
   (a7) an antibody variable region that comprises a heavy chain complementarity determining region 1 (HCDR1) comprising an amino acid sequence that is at least 95% identical to SEQ ID NO: 35, a heavy chain complementarity determining region 2 (HCDR2) comprising an amino acid sequence that is at least 95% identical to SEQ ID NO: 47, a heavy chain complementarity determining region 3 (HCDR3) comprising an amino acid sequence that is at least 95% identical to SEQ ID NO: 59, a light chain complementarity determining region 1 (LCDR1) comprising an amino acid sequence that is at least 95% identical to SEQ ID NO: 71, a light chain complementarity determining region 2 (LCDR2) comprising an amino acid sequence that is at least 95% identical to SEQ ID NO: 83, and a light chain complementarity determining region 3 (LCDR3) comprising an amino acid sequence that is at least 95% identical to SEQ ID NO: 95;
   (a8) an antibody variable region that comprises a heavy chain complementarity determining region 1 (HCDR1) comprising an amino acid sequence that is at least 95% identical to SEQ ID NO: 36, a heavy chain complementarity determining region 2 (HCDR2) comprising an amino acid sequence that is at least 95% identical to SEQ ID NO: 48, a heavy chain complementarity determining region 3 (HCDR3) comprising an amino acid sequence that is at least 95% identical to SEQ ID NO: 60, a light chain complementarity determining region 1 (LCDR1) comprising an amino acid sequence that is at least 95% identical to SEQ ID NO: 72, a light chain complementarity determining region 2 (LCDR2) comprising an amino acid sequence that is at least 95% identical to SEQ ID NO: 84, and a light chain complementarity determining region 3 (LCDR3) comprising an amino acid sequence that is at least 95% identical to SEQ ID NO: 96;
   (a9) an antibody variable region that comprises a heavy chain complementarity determining region 1 (HCDR1) comprising an amino acid sequence that is at least 95% identical to SEQ ID NO: 37, a heavy chain complementarity determining region 2 (HCDR2) comprising an amino acid sequence that is at least 95% identical to SEQ ID NO: 49, a heavy chain complementarity determining region 3 (HCDR3) comprising an amino acid sequence that is at least 95% identical to SEQ ID NO: 61, a light chain complementarity determining region 1 (LCDR1) comprising an amino acid sequence that is at least 95% identical to SEQ ID NO: 73, a light chain complementarity determining region 2 (LCDR2) comprising an amino acid sequence that is at least 95% identical to SEQ ID NO: 85, and a light chain complementarity determining region 3 (LCDR3) comprising an amino acid sequence that is at least 95% identical to SEQ ID NO: 97;
   (a10) an antibody variable region that comprises a heavy chain complementarity determining region 1 (HCDR1) comprising an amino acid sequence that is at least 95% identical to SEQ ID NO: 38, a heavy chain complementarity determining region 2 (HCDR2) comprising an amino acid sequence that is at least 95% identical to SEQ ID NO: 50, a heavy chain complementarity determining region 3 (HCDR3) comprising an amino acid sequence that is at least 95% identical to SEQ ID NO: 62, a light chain complementarity determining region 1 (LCDR1) comprising an amino acid sequence that is at least 95% identical to SEQ ID NO: 74, a light chain complementarity determining region 2 (LCDR2) comprising an amino acid sequence that is at least 95% identical to SEQ ID NO: 86, and a light chain complementarity determining region 3 (LCDR3) comprising an amino acid sequence that is at least 95% identical to SEQ ID NO: 98;
   (a11) an antibody variable region that comprises a heavy chain complementarity determining region 1 (HCDR1) comprising an amino acid sequence that is at least 95% identical to SEQ ID NO: 39, a heavy chain complementarity determining region 2 (HCDR2) comprising an amino acid sequence that is at least 95% identical to SEQ ID NO: 51, a heavy chain complementarity determining region 3 (HCDR3) comprising an amino acid sequence that is at least 95% identical to SEQ ID NO: 63, a light chain complementarity determining region 1 (LCDR1) comprising an amino acid sequence that is at least 95% identical to SEQ ID NO: 75, a light chain complementarity determining region 2 (LCDR2) comprising an amino acid sequence that is at least 95% identical to SEQ ID NO: 87, and a light chain complementarity determining region 3 (LCDR3) comprising an amino acid sequence that is at least 95% identical to SEQ ID NO: 99;
   (a12) an antibody variable region that comprises a heavy chain complementarity determining region 1 (HCDR1) comprising an amino acid sequence that is at least 95% identical to SEQ ID NO: 40, a heavy chain complementarity determining region 2 (HCDR2) comprising an amino acid sequence that is at least 95% identical to SEQ ID NO: 52, a heavy chain complementarity determining region 3 (HCDR3) comprising an amino acid sequence that is at least 95% identical to SEQ ID NO: 64, a light chain complementarity determining region 1 (LCDR1) comprising an amino acid sequence that is at least 95% identical to SEQ ID NO: 76, a light chain complementarity determining region 2 (LCDR2) comprising an amino acid sequence that is at least 95% identical to SEQ ID NO: 88, and a light chain complementarity determining region 3 (LCDR3) comprising an amino acid sequence that is at least 95% identical to SEQ ID NO: 100;
   (b1) an antibody variable region that comprises a HCDR1 comprising an amino acid sequence of SEQ ID NO: 29, a HCDR2 comprising an amino acid sequence of SEQ ID NO: 41, a HCDR3 comprising an amino acid sequence of SEQ ID NO: 53, a LCDR1 comprising an amino acid sequence of SEQ ID NO: 65, a LCDR2 comprising an amino acid sequence of SEQ ID NO: 77, and a LCDR3 comprising an amino acid sequence of SEQ ID NO: 89;
   (b2) an antibody variable region that comprises a HCDR1 comprising an amino acid sequence of SEQ ID NO: 30, a HCDR2 comprising an amino acid sequence of SEQ ID NO: 42, a HCDR3 comprising an amino acid sequence of SEQ ID NO: 54, a LCDR1 comprising an amino acid sequence of SEQ ID NO: 66, a LCDR2 comprising an amino acid sequence of SEQ ID NO: 78, and a LCDR3 comprising an amino acid sequence of SEQ ID NO: 90;
   (b3) an antibody variable region that comprises a HCDR1 comprising an amino acid sequence of SEQ ID NO: 31, a HCDR2 comprising an amino acid sequence of SEQ ID NO: 43, a HCDR3 comprising an amino acid sequence of SEQ ID NO: 55, a LCDR1 comprising an amino acid sequence of SEQ ID NO: 67, a LCDR2 comprising an amino acid sequence of SEQ ID NO: 79, and a LCDR3 comprising an amino acid sequence of SEQ ID NO: 91;
   (b4) an antibody variable region that comprises a HCDR1 comprising an amino acid sequence of SEQ ID NO: 32, a HCDR2 comprising an amino acid sequence of SEQ ID NO: 44, a HCDR3 comprising an amino acid sequence of SEQ ID NO: 56, a LCDR1 comprising an amino acid sequence of SEQ ID NO: 68, a LCDR2 comprising an amino acid sequence of SEQ ID NO: 80, and a LCDR3 comprising an amino acid sequence of SEQ ID NO: 92;
   (b5) an antibody variable region that comprises a HCDR1 comprising an amino acid sequence of SEQ ID NO: 33, a HCDR2 comprising an amino acid sequence of SEQ ID NO: 45, a HCDR3 comprising an amino acid sequence of SEQ ID NO: 57, a LCDR1 comprising an amino acid sequence of SEQ ID NO: 69, a LCDR2 comprising an amino acid sequence of SEQ ID NO: 81, and a LCDR3 comprising an amino acid sequence of SEQ ID NO: 93;
   (b6) an antibody variable region that comprises a HCDR1 comprising an amino acid sequence of SEQ ID NO: 34, a HCDR2 comprising an amino acid sequence of SEQ ID NO: 46, a HCDR3 comprising an amino acid sequence of SEQ ID NO: 58, a LCDR1 comprising an amino acid sequence of SEQ ID NO: 70, a LCDR2 comprising an amino acid sequence of SEQ ID NO: 82, and a LCDR3 comprising an amino acid sequence of SEQ ID NO: 94;
   (b7) an antibody variable region that comprises a HCDR1 comprising an amino acid sequence of SEQ ID NO: 35, a HCDR2 comprising an amino acid sequence of SEQ ID NO: 47, a HCDR3 comprising an amino acid sequence of SEQ ID NO: 59, a LCDR1 comprising an amino acid sequence of SEQ ID NO: 71, a LCDR2 comprising an amino acid sequence of SEQ ID NO: 83, and a LCDR3 comprising an amino acid sequence of SEQ ID NO: 95;
   (b8) an antibody variable region that comprises a HCDR1 comprising an amino acid sequence of SEQ ID NO: 36, a HCDR2 comprising an amino acid sequence of SEQ ID NO: 48, a HCDR3 comprising an amino acid sequence of SEQ ID NO: 60, a LCDR1 comprising an amino acid sequence of SEQ ID NO: 72, a LCDR2 comprising an amino acid sequence of SEQ ID NO: 84, and a LCDR3 comprising an amino acid sequence of SEQ ID NO: 96;
   (b9) an antibody variable region that comprises a HCDR1 comprising an amino acid sequence of SEQ ID NO: 37, a HCDR2 comprising an amino acid sequence of SEQ ID NO: 49, a HCDR3 comprising an amino acid sequence of SEQ ID NO: 61, a LCDR1 comprising an amino acid sequence of SEQ ID NO: 73, a LCDR2 comprising an amino acid sequence of SEQ ID NO: 85, and a LCDR3 comprising an amino acid sequence of SEQ ID NO: 97;
   (b10) an antibody variable region that comprises a HCDR1 comprising an amino acid sequence of SEQ ID NO: 38, a HCDR2 comprising an amino acid sequence of SEQ ID NO: 50, a HCDR3 comprising an amino acid sequence of SEQ ID NO: 62, a LCDR1 comprising an amino acid sequence of SEQ ID NO: 74, a LCDR2 comprising an amino acid sequence of SEQ ID NO: 86, and a LCDR3 comprising an amino acid sequence of SEQ ID NO: 98;
   (b11) an antibody variable region that comprises a HCDR1 comprising an amino acid sequence of SEQ ID NO: 39, a HCDR2 comprising an amino acid sequence of SEQ ID NO: 51, a HCDR3 comprising an amino acid sequence of SEQ ID NO: 63, a LCDR1 comprising an amino acid sequence of SEQ ID NO: 75, a LCDR2 comprising an amino acid sequence of SEQ ID NO: 87, and a LCDR3 comprising an amino acid sequence of SEQ ID NO: 99;
   (b12) an antibody variable region that comprises a HCDR1 comprising an amino acid sequence of SEQ ID NO: 40, a HCDR2 comprising an amino acid sequence of SEQ ID NO: 52, a HCDR3 comprising an amino acid sequence of SEQ ID NO: 64, a LCDR1 comprising an amino acid sequence of SEQ ID NO: 76, a LCDR2 comprising an amino acid sequence of SEQ ID NO: 88, and a LCDR3 comprising an amino acid sequence of SEQ ID NO: 100;
   (c1) an antibody variable region that comprises a heavy chain variable domain (VH) of SEQ ID NO: 5, and a light chain variable domain (VL) of SEQ ID NO: 17;
   (c2) an antibody variable region that comprises a heavy chain variable domain (VH) of SEQ ID NO: 6, and a light chain variable domain (VL) of SEQ ID NO: 18;
   (c3) an antibody variable region that comprises a heavy chain variable domain (VH) of SEQ ID NO: 7, and a light chain variable domain (VL) of SEQ ID NO: 19;
   (c4) an antibody variable region that comprises a heavy chain variable domain (VH) of SEQ ID NO: 8, and a light chain variable domain (VL) of SEQ ID NO: 20;
   (c5) an antibody variable region that comprises a heavy chain variable domain (VH) of SEQ ID NO: 9, and a light chain variable domain (VL) of SEQ ID NO: 21;
   (c6) an antibody variable region that comprises a heavy chain variable domain (VH) of SEQ ID NO: 10, and a light chain variable domain (VL) of SEQ ID NO: 22;
   (c7) an antibody variable region that comprises a heavy chain variable domain (VH) of SEQ ID NO: 11, and a light chain variable domain (VL) of SEQ ID NO: 23;
   (c8) an antibody variable region that comprises a heavy chain variable domain (VH) of SEQ ID NO: 12, and a light chain variable domain (VL) of SEQ ID NO: 24;
   (c9) an antibody variable region that comprises a heavy chain variable domain (VH) of SEQ ID NO: 13, and a light chain variable domain (VL) of SEQ ID NO: 25;
   (c10) an antibody variable region that comprises a heavy chain variable domain (VH) of SEQ ID NO: 14, and a light chain variable domain (VL) of SEQ ID NO: 26;
   (c11) an antibody variable region that comprises a heavy chain variable domain (VH) of SEQ ID NO: 15, and a light chain variable domain (VL) of SEQ ID NO: 27;
   (c12) an antibody variable region that comprises a heavy chain variable domain (VH) of SEQ ID NO: 16, and a light chain variable domain (VL) of SEQ ID NO: 28;
   (d) an antibody variable region that competes for binding to HER2 with any one of the antibody variable regions of (a1) to (c12);
   (e) an antibody variable region that binds to the same epitope on HER2 with any one of the antibody variable regions of (a1) to (c12); and preferably further comprises:
[B] a second antigen-binding domain comprising any one of the following antibody variable regions:
   (a) an antibody variable region that comprises a HCDR1 comprising an amino acid sequence of SEQ ID NO: 103, a HCDR2 comprising an amino acid sequence of SEQ ID NO: 104, a HCDR3 comprising an amino acid sequence of SEQ ID NO: 105, a LCDR1 comprising an amino acid sequence of SEQ ID NO: 106, a LCDR2 comprising an amino acid sequence of SEQ ID NO: 107, and a LCDR3 comprising an amino acid sequence of SEQ ID NO: 108;
   (b) an antibody variable region that comprises a heavy chain variable domain (VH) of SEQ ID NO: 101, and a light chain variable domain (VL) of SEQ ID NO: 102.

In one preferred embodiment, the antigen-binding molecule of the present invention is a bispecific antibody that specifically binds to human HER2 S310 and CD3.

In one embodiment, the antigen-binding molecule of the present invention comprises a first antigen-binding domain which has at least 1-time, 2-time, 3-time, 4-time, 5-time, 10-time, 20-time, 50-time, 100-time, 1000-time, 10000-time stronger binding activity to human HER2 S310F, as compared to any of the reference antibody as described in Table 2 or 3. Preferably, the binding activity is measured by SPR at the following condition: 37 degrees C., pH 7.4, 20 mM ACES, 150 mM NaCl, 0.05% Tween 20, 0.005% NaN3; the antibody is immobilized on a CM4 sensor chip, the antigen serves as analyte. In an embodiment, the antigen-binding molecule of the present invention, which is specific to the HER2 S310F mutant, comprises a first antigen-binding domain which has a smaller KD ratio of the KD value for binding to human HER2 S310F to the KD value for binding to human wild type HER2 (i.e. KD_{HER2 S310F}/KD_{wild type HER2}), compared to a reference antibody, preferably a reference antibody as described in Table 2 or 3. In a preferred embodiment, the antigen-binding molecule of the present invention comprises a first antigen-binding domain which has at least 10-fold, 100-fold, 1,000-fold or 10,000-fold, or 100,000-fold smaller (i.e., 1/10, 1/100, 1/1000, 1/10000, or 1/100000) KD ratio of the KD value for binding to human HER2 S310F to the KD value for binding to human wild type HER2 (i.e. KD_{HER2 S310F}/KD_{wild type HER2}), compared to any of the reference antibody as described in Table 2 or 3. Preferably, the binding activity is measured by SPR at the following condition: 37 degrees C., pH 7.4, 20 mM ACES, 150 mM NaCl, 0.05% Tween 20, 0.005% NaN3; the antigen-binding molecule is immobilized on a CM4 sensor chip, the antigen serves as analyte.

In one aspect, the present invention provides a human T cell comprising a nucleic acid sequence encoding a chimeric antigen receptor (CAR). In some embodiments, a CAR of the present invention comprises the first antigen binding domain of the present invention, preferably one of the antigen-binding domain as described in Table 2 or 3. In further embodiments, a CAR of the present invention comprises the first antigen binding domain and a transmembrane domain, and an intracellular domain and a signaling domain of a costimulatory molecule. In some embodiments, the intracellular domain may include a CD3-zeta signaling domain. In some embodiments, the antigen binding fragment is a scFv. In some embodiments, the T cell comprises a vector that comprises the nucleic acid sequence. For example, the vector is a lentiviral vector. In another aspect, the present invention provides a pharmaceutical composition comprising the human T cell as described above.

### BRIEF DESCRIPTION OF DRAWINGS

FIG. 1 Binding analysis of the anti-HER2 S310F monospecific antibodies to transfectants.

FIG. 2 Binding analysis of the anti-HER2 S310F monospecific antibodies to cancer cell lines.

FIG. 3 T cell activation activity of anti-HER2 S310F/CD3 bispecific antibodies in the presence of HER2 S310F or wild type HER2 expressing transfectants.

FIG. 4 T-cell-dependent cell cytotoxicity of anti-HER2 S310F/CD3 bispecific antibodies against HER2 S310F or wild type HER2 expressing transfectants.

FIG. 5 T-cell-dependent cell cytotoxicity of anti-HER2 S310F/CD3 bispecific antibodies against cancer cell lines.

### DESCRIPTION OF EMBODIMENTS

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993).

The definitions and detailed description below are provided to facilitate understanding of the present invention illustrated herein.

### Amino Acids

Herein, amino acids are described by one- or three-letter codes or both, for example, Ala/A, Leu/L, Arg/R, Lys/K, Asn/N, Met/M, Asp/D, Phe/F, Cys/C, Pro/P, Gln/Q, Ser/S, Glu/E, Thr/T, Gly/G, Trp/W, His/H, Tyr/Y, Ile/I, or Val/V.

### Alteration of Amino Acids

For amino acid alteration in the amino acid sequence of an antigen-binding molecule, known methods such as site-directed mutagenesis methods (Kunkel et al. (Proc. Natl. Acad. Sci. USA (1985) 82, 488-492)) and overlap extension PCR may be appropriately employed. Furthermore, several known methods may also be employed as amino acid alteration methods for substitution to non-natural amino acids (Annu Rev. Biophys. Biomol. Struct. (2006) 35, 225-249; and Proc. Natl. Acad. Sci. U.S.A. (2003) 100 (11), 6353-6357). For example, it is suitable to use a cell-free translation system (Clover Direct (Protein Express)) containing a tRNA which has a non-natural amino acid bound to a complementary amber suppressor tRNA of one of the stop codons, the UAG codon (amber codon).

In the present specification, the meaning of the term “and/or” when describing the site of amino acid alteration includes every combination where “and” and “or” are suitably combined. Specifically, for example, “the amino acids at positions 33, 55, and/or 96 are substituted” includes the following variation of amino acid alterations: amino acid(s) at (a) position 33, (b) position 55, (c) position 96, (d) positions 33 and 55, (e) positions 33 and 96, (f) positions 55 and 96, and (g) positions 33, 55, and 96.

Furthermore, herein, as an expression showing alteration of amino acids, an expression that shows before and after a number indicating a specific position, one-letter or three-letter codes for amino acids before and after alteration, respectively, may be used appropriately. For example, the alteration N100bL or Asn100bLeu used when substituting an amino acid contained in an antibody variable region indicates substitution of Asn at position 100b (according to Kabat numbering) with Leu. That is, the number shows the amino acid position according to Kabat numbering, the one-letter or three-letter amino-acid code written before the number shows the amino acid before substitution, and the one-letter or three-letter amino-acid code written after the number shows the amino acid after substitution. Similarly the alteration P238D or Pro238Asp used when substituting an amino acid of the Fc region contained in an antibody constant region indicates substitution of Pro at position 238 (according to EU numbering) with Asp. That is, the number shows the amino acid position according to EU numbering, the one-letter or three-letter amino-acid code written before the number shows the amino acid before substitution, and the one-letter or three-letter amino-acid code written after the number shows the amino acid after substitution.

### Antigen-Binding Molecule

The term “antigen-binding molecules”, as used herein, refers to any molecule that comprises an antigen-binding domain or any molecule that has binding activity to an antigen, and may further refers to molecules such as a peptide or protein having a length of about five amino acids or more. The peptide and protein are not limited to those derived from a living organism, and for example, they may be a polypeptide produced from an artificially designed sequence. They may also be any of a naturally-occurring polypeptide, synthetic polypeptide, recombinant polypeptide, and such.

A favorable example of an antigen-binding molecule of the present invention is an antigen-binding molecule that comprises a plurality of antigen-binding domains. In certain embodiments, the antigen-binding molecule of the present invention is an antigen-binding molecule that comprises two antigen-binding domains with different antigen-binding specificities. In certain embodiments, the antigen-binding molecule of the present invention is an antigen-binding molecule that comprises two antigen-binding molecules comprising two antigen-binding domains with different antigen-binding specificities, and an FcRn-binding domain contained in an antibody Fc region. As a method for extending the blood half-life of a protein administered to a living body, the method of adding an FcRn-binding domain of an antibody to the protein of interest and utilizing the function of FcRn-mediated recycling is well known.

### Antigen-Binding Domain

The term “antigen-binding domain”, as used herein, refers to an antibody portion which comprises a region that specifically binds and is complementary to the whole or a portion of an antigen. When the molecular weight of an antigen is large, an antibody can only bind to a particular portion of the antigen. The particular portion is called “epitope”. An antigen-binding domain can be provided from one or more antibody variable domains. Preferably, the antigen-binding domains contain antibody variable region that comprising both the antibody light chain variable region (VL) and antibody heavy chain variable region (VH). Such preferable antigen-binding domains include, for example, “single-chain Fv (scFv)”, “single-chain antibody”, “Fv”, “single-chain Fv2 (scFv2)”, “Fab”, “F(ab′)2”, and VHH (Variable domain of Heavy chain of Heavy chain [antibody]) structure.

The antigen-binding domain of an antigen-binding molecule of the present invention “specifically binds to HER2 S310F or a T cell receptor complex molecule”. That is, HER2 S310F and a T cell receptor complex molecule are respectively preferable antigens of interest—the antigen-binding domain has binding activity to its antigen of interest, but which do not substantially recognize and bind other molecules in a sample containing a mixed population of antigenic molecules. As used herein, the phrase “has binding activity” refers to the activity of an antigen-binding domain, antibody, antigen-binding molecule, antibody variable fragment, or such (hereinafter, “antigen-binding domain or such”) to bind to an antigen of interest at a level of specific binding higher than the level of non-specific or background binding. In other words, such an antigen-binding domain or such “has a specific/significant binding activity” towards the antigen of interest. The specificity can be measured by any methods for detecting affinity or binding activity as mentioned herein or known in the art. The above-mentioned level of specific binding may be high enough to be recognized by a skilled person as being significant. For example, when a skilled person can detect or observe any significant or relatively strong signals or values of binding between the antigen-binding domain or such and the antigen of interest in a suitable binding assay, it can be said that the antigen-binding domain or such has a “specific/significant binding activity” towards the antigen of interest. Alternatively, “have a specific/significant binding activity” can be rephrased as “specifically/significantly bind” (to the antigen of interest). Sometimes, the phrase “having binding activity” has substantially the same meaning as the phrase “having a specific/significant binding activity” in the art. As used herein, an antigen-binding molecule or an antibody that “specifically binds” to an antigen refers to antigen-binding molecule or an antibody that binds to the antigen and substantially identical antigens with high affinity, which means having a KD of 10-7 M or less, preferably 10-8 M or less, even more preferably 10-9 M or less, and most preferably between 10-8 M and 10-10 M or less, but does not bind with high affinity to unrelated antigens, as measured by a surface plasmon resonance assay or a cell binding assay.

In some embodiments, binding activity or affinity of the antigen-binding domains of the present invention (e.g., anti-HER2 S310F/CD3 bispecific antibody) to the antigen of interest (e.g., human HER2 S310F) are assessed at 37 degrees C. using e.g., Biacore T200 instrument (GE Healthcare) or Biacore 8K instrument (GE Healthcare). Anti-human Fc (e.g., GE Healthcare) is immobilized onto all flow cells of a CM4 sensor chip using amine coupling kit (e.g, GE Healthcare). The antigen-binding molecules or antigen-binding domains are captured onto the anti-Fc sensor surfaces, then the antigen (e.g. HER2 S310F or CD3) is injected over the flow cell. The capture levels of the molecules/domains may be aimed at 200 resonance unit (RU). Recombinant human HER2 S310F or wild-type HER2 may be injected at 400 to 25 nM prepared by two-fold serial dilution, followed by dissociation. All antigen-binding domains and analytes are prepared in ACES pH 7.4 containing 20 mM ACES, 150 mM NaCl, 0.05% Tween 20, 0.005% NaN3. Sensor surface is regenerated each cycle with 3 M MgCl2. Binding affinity are determined by processing and fitting the data to 1:1 binding model using e.g., Biacore T200 Evaluation software, version 2.0 (GE Healthcare) or Biacore 8K Evaluation software (GE Healthcare). The KD values for HER2 S310F and wild-type HER2 are determined, and the KD ratio (i.e., KDHER2 S310F/KDwild type HER2) is calculated for assessing the specific binding activity or affinity of the antigen-binding domains of the present invention.

### HER2 and HER2 S310F

The term “HER2” (also known as Proto-oncogene Neu, ErbB-2, Tyrosine kinase-type cell surface receptor HER2, CD340) in the context of “wild type HER2”, as used herein, refers to any native HER2 isoforms from human, including as disclosed in RefSeq accession numbers NP_001005862.1 (receptor tyrosine-protein kinase erbB-2 isoform b; SEQ ID NO: 162), NP_001276865.1 (receptor tyrosine-protein kinase erbB-2 isoform c; SEQ ID NO: 163), NP_001276866.1 (receptor tyrosine-protein kinase erbB-2 isoform d; SEQ ID NO: 164), NP_001276867.1 (receptor tyrosine-protein kinase erbB-2 isoform e; SEQ ID NO: 165), NP_004439.2 (receptor tyrosine-protein kinase erbB-2 isoform a; SEQ ID NO: 166). The HER2 S310F protein can dimerize in a ligand-independent manner to transmit signals to downstream signal transduction pathways. In some embodiments, the present invention provides an antigen-binding molecule or antigen-binding molecule that prevents ligand-independent dimerization of a human HER2 S310F mutant.

Herein, the term “HER2 S310F” refers to any HER2 mutant with a Serine to Phenylalanine mutation at the amino acid residue position corresponding to the 310th position of NP_004439.2 (receptor tyrosine-protein kinase erbB-2 isoform a; SEQ ID NO: 166).

```
NP_004439.2 (receptor tyrosine-protein
kinase erbB-2 isoform a; SEQ ID NO: 166)
                10 20 30 40
MELAALCRWG LLLALLPPGA ASTQVCTGTD MKLRLPASPE
                50 60 70 80
THLDMLRHLY QGCQVVQGNL ELTYLPTNAS LSFLQDIQEV
                90 100 110 120
QGYVLIAHNQ VRQVPLQRLR IVRGTQLFED NYALAVLDNG
              130 140 150 160
DPLNNTTPVT GASPGGLREL QLRSLTEILK GGVLIQRNPQ
              170 180 190 200
LCYQDTILWK DIFHKNNQLA LTLIDTNRSR ACHPCSPMCK
              210 220 230 240
GSRCWGESSE DCQSLTRTVC AGGCARCKGP LPTDCCHEQC
              250 260 270 280
AAGCTGPKHS DCLACLHFNH SGICELHCPA LVTYNTDTFE
              290 300 310 320
SMPNPEGRYT FGASCVTACP YNYLSTDVGS CTLVCPLHNQ
              330 340 350 360
EVTAEDGTQR CEKCSKPCAR VCYGLGMEHL REVRAVTSAN
              370 380 390 400
IQEFAGCKKI FGSLAFLPES FDGDPASNTA PLQPEQLQVF
              410 420 430 440
ETLEEITGYL YISAWPDSLP DLSVFQNLQV IRGRILHNGA
              450 460 470 480
YSLTLQGLGI SWLGLRSLRE LGSGLALIHH NTHLCFVHTV
              490 500 510 520
PWDQLFRNPH QALLHTANRP EDECVGEGLA CHQLCARGHC
              530 540 550 560
WGPGPTQCVN CSQFLRGQEC VEECRVLQGL PREYVNARHC
              570 580 590 600
LPCHPECQPQ NGSVTCFGPE ADQCVACAHY KDPPFCVARC
              610 620 630 640
PSGVKPDLSY MPIWKFPDEE GACQPCPINC THSCVDLDDK
              650 660 670 680
GCPAEQRASP LTSIISAVVG ILLVVVLGVV FGILIKRRQQ
              690 700 710 720
KIRKYTMRRL LQETELVEPL TPSGAMPNQA QMRILKETEL
              730 740 750 760
RKVKVLGSGA FGTVYKGIWI PDGENVKIPV AIKVLRENTS
              770 780 790 800
PKANKEILDE AYVMAGVGSP YVSRLLGICL TSTVQLVTQL
              810 820 830 840
MPYGCLLDHV RENRGRLGSQ DLLNWCMQIA KGMSYLEDVR
              850 860 870 880
LVHRDLAARN VLVKSPNHVK ITDFGLARLL DIDETEYHAD
              890 900 910 920
GGKVPIKWMA LESILRRRFT HQSDVWSYGV TVWELMTFGA
              930 940 950 960
KPYDGIPARE IPDLLEKGER LPQPPICTID VYMIMVKCWM
              970 980 990 1000
IDSECRPRFR ELVSEFSRMA RDPQRFVVIQ NEDLGPASPL
            1010 1020 1030 1040
DSTFYRSLLE DDDMGDLVDA EEYLVPQQGF FCPDPAPGAG
            1050 1060 1070 1080
GMVHHRHRSS STRSGGGDLT LGLEPSEEEA PRSPLAPSEG
            1090 1100 1110 1120
AGSDVFDGDL GMGAAKGLQS LPTHDPSPLQ RYSEDPTVPL
            1130 1140 1150 1160
PSETDGYVAP LTCSPQPEYV NQPDVRPQPP SPREGPLPAA
            1170 1180 1190 1200
RPAGATLERP KTLSPGKNGV VKDVFAFGGA VENPEYLTPQ
            1210 1220 1230 1240
GGAAPQPHPP PAFSPAFDNL YYWDQDPPER GAPPSTFKGT
            1250
PTAENPEYLG LDVPV
```

### Affinity/Binding Activity

“Affinity” or “binding activity” refers to the strength of the sum total of noncovalent interactions between a single binding site of a molecule (e.g., an antigen-binding molecule or antibody) and its binding partner (e.g., an antigen). Unless indicated otherwise, as used herein, “binding affinity/activity” refers to intrinsic binding affinity which reflects a 1:1 interaction between members of a binding pair (e.g., antigen-binding molecule and antigen, or antibody and antigen). The affinity of a molecule X for its partner Y can generally be represented by the dissociation constant (KD). Affinity can be measured by common methods known in the art, including those described herein. Specific illustrative and exemplary embodiments for measuring binding affinity are described in the following.

Herein, “no binding activity” (or “no specific binding activity”) refers to activity of an antibody to bind to an antigen of no interest at a level of binding that includes non-specific or background binding but does not include specific binding. In other words, such an antibody “does not bind to” (or “does not specifically bind to”) the antigen of no interest. The specificity can be measured by any assay methods mentioned in this specification or known in the art, by determining the KD ratio between the antigen of interest and the antigen of no interest. The above-mentioned level of non-specific or background binding may be zero, or may not be zero but near zero, or may be very low enough to be technically neglected by those skilled in the art. For example, when a skilled person cannot detect or observe any significant (or relatively strong) signal for binding between the antibody and the antigen of no interest in a suitable binding assay, it can be said that the antibody “has no (specific) binding activity” or “does not (specifically) bind to” the antigen of no interest. Meanwhile, “weaker binding activity” refers to activity of an antibody to bind to an antigen of interest at a level that is lower than the level of binding of a certain reference (or control) antibody to the antigen. The degree of “weakness” may be measured by any assay methods mentioned herein or known in the art, for example, by determining a difference in the KD value or ratio between the antibody of interest and the reference/control antibody.

### Methods to Determine Affinity

In certain embodiments, the antigen-binding domain of an antigen-binding molecule or antibody provided herein has a dissociation constant (KD) of 1 micro M or less, 120 nM or less, 100 nM or less, 80 nM or less, 70 nM or less, 50 nM or less, 40 nM or less, 30 nM or less, 20 nM or less, 10 nM or less, 2 nM or less, 1 nM or less, 0.1 nM or less, 0.01 nM or less, or 0.001 nM or less (e.g., 10-8 M or less, 10-8 M to 10-13 M, 10-9 M to 10-13 M) for its antigen. In certain embodiments, the KD value of the first antigen-binding domain of the antibody/antigen-binding molecule for HER2 S310F falls within the range of 1-40, 1-50, 1-70, 1-80, 30-50, 30-70, 30-80, 40-70, 40-80, or 60-80 nM.

In one embodiment, KD is measured by a radiolabeled antigen binding assay (RIA). In one embodiment, an RIA is performed with the Fab version of an antibody of interest and its antigen. For example, solution binding affinity of Fabs for antigen is measured by equilibrating Fab with a minimal concentration of (125I)-labeled antigen in the presence of a titration series of unlabeled antigen, then capturing bound antigen with an anti-Fab antibody-coated plate (see, e.g., Chen et al., J. Mol. Biol. 293:865-881(1999)). To establish conditions for the assay, MICROTITER® multi-well plates (Thermo Scientific) are coated overnight with 5 micro g/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23 degrees C.). In a non-adsorbent plate (Nunc #269620), 100 pM or 26 pM [125I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20@) in PBS. When the plates have dried, 150 micro 1/well of scintillant (MICROSCINT-20®; Packard) is added, and the plates are counted on a TOPCOUNT® gamma counter (Packard) for ten minutes. Concentrations of each Fab that give less than or equal to 20% of maximal binding are chosen for use in competitive binding assays.

According to another embodiment, KD is measured using a BIACORE® surface plasmon resonance assay. For example, an assay using a BIACORE®-2000 or a BIACORE®-3000 (BIAcore, Inc., Piscataway, NJ) is performed at 25 degrees C. with immobilized antigen CM5 chips at approximately 10 response units (RU). In one embodiment, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 micro g/ml (approximately 0.2 micro M) before injection at a flow rate of 5 micro 1/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20®) surfactant (PBST) at 25 degrees C. at a flow rate of approximately 25 micro 1/min. Association rates (kon) and dissociation rates (koff) are calculated using a simple one-to-one Langmuir binding model (BIACORE®) Evaluation Software version 3.2) by simultaneously fitting the association and dissociation sensorgrams. The equilibrium dissociation constant (KD) is calculated as the ratio koff/kon. See, e.g., Chen et al., J. Mol. Biol. 293:865-881 (1999). If the on-rate exceeds 106 M-1 s-1 by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25 degrees C. of a 20 nM anti-antigen antibody (Fab form) in PBS, pH 7.2, in the presence of increasing concentrations of antigen as measured in a spectrometer, such as a stop-flow equipped spectrophotometer (Aviv Instruments) or a 8000-series SLM-AMINCO® spectrophotometer (ThermoSpectronic) with a stirred cuvette.

Methods for measuring the affinity of the antigen-binding domain of an antibody are described above, and one skilled in art can carry out affinity measurement for other antigen-binding domains.

### Antibody

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

### Class of Antibody

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgAQ1, and IgA2. The heavy chain constant domains that correspond to the different classes of immunoglobulins are called alpha, delta, epsilon, gamma, and mu, respectively.

### Framework

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

### Human Consensus Framework

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda MD (1991), vols. 1-3. In one embodiment, for the VL, the subgroup is subgroup kappa I as in Kabat et al., supra. In one embodiment, for the VH, the subgroup is subgroup III as in Kabat et al., supra.

### HVR

The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise six HVRs: three in the VH (H1, H2, H3), and three in the VL (L1, L2, L3). Exemplary HVRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991));
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)); and
(d) combinations of (a), (b), and/or (c), including HVR amino acid residues 46-56 (L2), 47-56 (L2), 48-56 (L2), 49-56 (L2), 26-35 (H1), 26-35b (H1), 49-65 (H2), 93-102 (H3), and 94-102 (H3).

Unless otherwise indicated, HVR residues and other residues in the variable domain (e.g., FR residues) are numbered herein according to Kabat et al., supra.

HVR-H1, HVR-H2, HVR-H3, HVR-L1, HVR-L2, and HVR-L3 is also mentioned as “HCDR1”, “HCDR2”, “HCDR3”, LCDR1”, “LCDR2”, and “LCDR3” respectively.

### Variable Region

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6th ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

### Identity (Sequence Identity)

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, Megalign (DNASTAR) software, or GENETYX® (Genetyx Co., Ltd.). Those skilled in the art can determine appropriate parameters for aligning sequences, including any algorithms needed to achieve maximal alignment over the full length of the sequences being compared.

The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, California, or may be compiled from the source code. The ALIGN-2 program should be compiled for use on a UNIX operating system, including digital UNIX V4.0D. All sequence comparison parameters are set by the ALIGN-2 program and do not vary. In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

### Chimeric Antibody

The term “chimeric” antibody refers to an antibody in which a portion of the heavy and/or light chain is derived from a particular source or species, while the remainder of the heavy and/or light chain is derived from a different source or species. Similarly, the term “chimeric antibody variable domain” refers to an antibody variable region in which a portion of the heavy and/or light chain variable region is derived from a particular source or species, while the remainder of the heavy and/or light chain variable region is derived from a different source or species.

### Humanized Antibody

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization. A “humanized antibody variable region” refers to the variable region of a humanized antibody.

### Human Antibody

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues. A “human antibody variable region” refers to the variable region of a human antibody.

### T Cell-Recruiting Antibody

A “T cell-recruiting (TR) antibody” has cytotoxicity by recruiting T cells as effector cells. Examples of TR antibodies include a bispecific antibody that recognizes and binds to (i) any one of the subunits forming a T-cell receptor complex on T-cells (i.e., “T-cell receptor complex molecule”), such as CD3, in particular a human CD3 epsilon chain, and (ii) an antigen on cancer cells. By binding to both the T-cell receptor complex molecule (such as CD3 or human CD3 epsilon chain) and the cancer antigen, the TR antibody recruits T cells and facilitates killing of cancer cells. In the context of the present specification, preferably, the antigen is a human HER2 S310F mutant.

Methods for Producing an Antibody with Desired Binding Activity

Methods for producing an antibody with desired binding activity are known to those skilled in the art. Below is an example that describes a method for producing an antibody (anti-HER2 S310F antibody) that binds to HER2 S310F. Antibodies that bind to a T-cell receptor complex and so on can also be produced according to the example described below.

Anti-HER2 S310F antibodies can be obtained as polyclonal or monoclonal antibodies using known methods. The anti-HER2 S310F antibodies preferably produced are monoclonal antibodies derived from mammals. Such mammal-derived monoclonal antibodies include antibodies produced by hybridomas or host cells transformed with an expression vector carrying an antibody gene by genetic engineering techniques.

Monoclonal antibody-producing hybridomas can be produced using known techniques, for example, as described below. Specifically, mammals are immunized by conventional immunization methods using a HER2 S310F protein as a sensitizing antigen. Resulting immune cells are fused with known parental cells by conventional cell fusion methods. Then, hybridomas producing an anti-HER2 S310F antibody can be selected by screening for monoclonal antibody-producing cells using conventional screening methods.

Specifically, monoclonal antibodies are prepared as mentioned below. First, a polypeptide comprising HER2 S310F can be synthesized or expressed which will be used as a sensitizing antigen or immunogen for antibody preparation. Alternatively, a nucleotide encoding the full length or preferably the extracellular domain (ECD) of HER2 S310F can be expressed to produce a HER2 S310F-containing protein. That is, a gene sequence encoding full-length HER2 S310F or HER2 S310F ECD is inserted into a known expression vector, and appropriate host cells are transformed with this vector. The desired human full-length HER2 S310F or HER2 S310F ECD protein is purified from the host cells or their culture supernatants by known methods.

The purified full-length HER2 S310F or HER2 S310F ECD protein can be used as a sensitizing antigen or immunogen for use in immunization of mammals. Partial peptides of full-length HER2 S310F or HER2 S310F ECD can also be used as sensitizing antigens. In this case, the partial peptides may also be obtained by chemical synthesis from the human HER2 amino acid sequence with the S310F substitution. Furthermore, they may also be obtained by incorporating a portion of the HER2 gene having S310F mutation into an expression vector and expressing it.

For sensitizing antigen, alternatively it is possible to use a fusion protein prepared by fusing a desired partial polypeptide or peptide of the full-length HER2 S310F or HER2 S310F ECD protein with a different polypeptide. For example, antibody Fc fragments and peptide tags are preferably used to produce fusion proteins to be used as sensitizing antigens. Vectors for expression of such fusion proteins can be constructed by fusing in frame genes encoding two or more desired polypeptide fragments and inserting the fusion gene into an expression vector as described above. Methods for producing fusion proteins are described in Molecular Cloning 2nd ed. (Sambrook, J et al., Molecular Cloning 2nd ed., 9.47-9.58 (1989) Cold Spring Harbor Lab. Press). Methods for preparing HER2 S310F to be used as a sensitizing antigen, and immunization methods using HER2 S310F are also described in the Examples of this specification later.

In one embodiment, synthesized polypeptide comprising amino acids 23-646 of human HER2 S310F (i.e. extracellular domain or ECD) with rabbit Fc tag on its C terminus (SEQ ID NO: 1 as shown below) was expressed transiently using the Expi293 cell line (Thermo Fisher), purified and used for immunization.

SEQ ID NO: 1
MGWSCIILFLVATATGVHSTQVCTGTDMKLRLPASPETHLDMLRHLYQGC
QVVQGNLELTYLPTNASLSFLQDIQEVQGYVLIAHNQVRQVPLQRLRIVR
GTQLFEDNYALAVLDNGDPLNNTTPVTGASPGGLRELQLRSLTEILKGGV
LIQRNPQLCYQDTILWKDIFHKNNQLALTLIDTNRSRACHPCSPMCKGSR
CWGESSEDCQSLTRTVCAGGCARCKGPLPTDCCHEQCAAGCTGPKHSDCL
ACLHFNHSGICELHCPALVTYNTDTFESMPNPEGRYTFGASCVTACPYNY
LSTDVGFCTLVCPLHNQEVTAEDGTQRCEKCSKPCARVCYGLGMEHLREV
RAVTSANIQEFAGCKKIFGSLAFLPESFDGDPASNTAPLQPEQLQVFETL
EEITGYLYISAWPDSLPDLSVFQNLQVIRGRILHNGAYSLTLQGLGISWL
GLRSLRELGSGLALIHHNTHLCFVHTVPWDQLERNPHQALLHTANRPEDE
CVGEGLACHQLCARGHCWGPGPTQCVNCSQFLRGQECVEECRVLQGLPRE
YVNARHCLPCHPECQPQNGSVTCFGPEADQCVACAHYKDPPFCVARCPSG
VKPDLSYMPIWKFPDEEGACQPCPINCTHSCVDLDDKGCPAEQGGGGSAP
STCSKPMCPPPELLGGPSVFIFPPKPKDTLMISRTPEVTCVVVDVSQDDP
EVQFTWYINNEQVRTARPPLREQQFNSTIRVVSTLPIAHQDWLRGKEFKC
KVHNKALPAPIEKTISKARGQPLEPKVYTMGPPREELSSRSVSLICMING
FYPSDISVEWEKNGKAEDNYKTTPTVLDSDGSYFLYSKLSVPTSEWQRGD
VFTCSVMHEALHNHYTQKSISRSPGK

As shown above, SEQ ID NO: 1 comprises the amino acid sequence of 23-646 of human HER2 S310F ECD (the F amino acid residue corresponding to position 310 of HER2 is bold and underlined) with linker (bold) followed by a rabbit Fc tag (underlined) on its C terminus including the signal sequence (italics, first 19 amino acid residues). The signal sequence is not part of the mature polypeptide chain.

There is no particular limitation on the mammals to be immunized with the sensitizing antigen. However, it is preferable to select the mammals by considering their compatibility with the parent cells to be used for cell fusion. In general, rodents such as mice, rats, and hamsters, rabbits, and monkeys are preferably used.

The above animals are immunized with a sensitizing antigen by known methods. Generally performed immunization methods include, for example, intraperitoneal or subcutaneous injection of a sensitizing antigen into mammals. Specifically, a sensitizing antigen is appropriately diluted with PBS (Phosphate-Buffered Saline), physiological saline, or the like. If desired, a conventional adjuvant such as Freund's complete adjuvant is mixed with the antigen, and the mixture is emulsified. Then, the sensitizing antigen is administered to a mammal several times at 4- to 21-day intervals. Appropriate carriers may be used in immunization with the sensitizing antigen. In particular, when a low-molecular-weight partial peptide is used as the sensitizing antigen, it is sometimes desirable to couple the sensitizing antigen peptide to a carrier protein such as albumin or keyhole limpet hemocyanin for immunization.

Alternatively, hybridomas producing a desired antibody can be prepared using DNA immunization as mentioned below. DNA immunization is an immunization method that confers immunostimulation by expressing a sensitizing antigen in an animal immunized as a result of administering a vector DNA constructed to allow expression of an antigen protein-encoding gene in the animal. As compared to conventional immunization methods in which a protein antigen is administered to animals to be immunized, DNA immunization is expected to be superior in that:
immunostimulation can be provided while retaining the structure of a membrane protein such as HER2 S310F; and
there is no need to purify the antigen for immunization.

In order to prepare a monoclonal antibody of the present invention using DNA immunization, first, a DNA expressing a HER2 S310F protein is administered to an animal to be immunized. The HER2 S310F-encoding DNA can be synthesized by known methods such as PCR. The obtained DNA is inserted into an appropriate expression vector, and then this is administered to an animal to be immunized. Preferably used expression vectors include, for example, commercially-available expression vectors such as pcDNA3.1. Vectors can be administered to an organism using conventional methods. For example, DNA immunization is performed by using a gene gun to introduce expression vector-coated gold particles into cells in the body of an animal to be immunized. Antibodies that recognized HER2 S310F can also be produced by the methods described in WO 2003/104453.

After immunizing a mammal as described above, an increase in the titer of a HER2 S310F-binding antibody is confirmed in the serum. Then, immune cells are collected from the mammal, and then subjected to cell fusion. In particular, splenocytes are preferably used as immune cells.

A mammalian myeloma cell is used as a cell to be fused with the above-mentioned immunocyte. The myeloma cells preferably comprise a suitable selection marker for screening. A selection marker confers characteristics to cells for their survival (or death) under a specific culture condition. Hypoxanthine-guanine phosphoribosyltransferase deficiency (hereinafter abbreviated as HGPRT deficiency) and thymidine kinase deficiency (hereinafter abbreviated as TK deficiency) are known as selection markers. Cells with HGPRT or TK deficiency have hypoxanthine-aminopterin-thymidine sensitivity (hereinafter abbreviated as HAT sensitivity). HAT-sensitive cells cannot synthesize DNA in a HAT selection medium, and are thus killed. However, when the cells are fused with normal cells, they can continue DNA synthesis using the salvage pathway of the normal cells, and therefore they can grow even in the HAT selection medium.

HGPRT-deficient and TK-deficient cells can be selected in a medium containing 6-thioguanine, 8-azaguanine (hereinafter abbreviated as 8AG), or 5′-bromodeoxyuridine, respectively. Normal cells are killed because they incorporate these pyrimidine analogs into their DNA. Meanwhile, cells that are deficient in these enzymes can survive in the selection medium, since they cannot incorporate these pyrimidine analogs. In addition, a selection marker referred to as G418 resistance provided by the neomycin-resistant gene confers resistance to 2-deoxystreptamine antibiotics (gentamycin analogs). Various types of myeloma cells that are suitable for cell fusion are known.

For example, myeloma cells including the following cells can be preferably used:
P3(P3x63Ag8.653) (J. Immunol. (1979) 123 (4), 1548-1550);
P3x63Ag8U.1 (Current Topics in Microbiology and Immunology (1978)81, 1-7);
NS-1 (C. Eur. J. Immunol. (1976)6 (7), 511-519);
MPC-11 (Cell (1976) 8 (3), 405-415);
SP2/0 (Nature (1978) 276 (5685), 269-270);
FO (J. Immunol. Methods (1980) 35 (1-2), 1-21);
S194/5.XX0.BU.1 (J. Exp. Med. (1978) 148 (1), 313-323);
R210 (Nature (1979) 277 (5692), 131-133), etc.

Cell fusions between the immunocytes and myeloma cells are essentially carried out using known methods, for example, a method by Kohler and Milstein et al. (Methods Enzymol. (1981) 73: 3-46).

More specifically, cell fusion can be carried out, for example, in a conventional culture medium in the presence of a cell fusion-promoting agent. The fusion-promoting agents include, for example, polyethylene glycol (PEG) and Sendai virus (HVJ). If required, an auxiliary substance such as dimethyl sulfoxide is also added to improve fusion efficiency.

The ratio of immunocytes to myeloma cells may be determined at one's own discretion, preferably, for example, one myeloma cell for every one to ten immunocytes. Culture media to be used for cell fusions include, for example, media that are suitable for the growth of myeloma cell lines, such as RPMI1640 medium and MEM medium, and other conventional culture medium used for this type of cell culture. In addition, serum supplements such as fetal calf serum (FCS) may be preferably added to the culture medium.

For cell fusion, predetermined amounts of the above immune cells and myeloma cells are mixed well in the above culture medium. Then, a PEG solution (for example, the average molecular weight is about 1,000 to 6,000) prewarmed to about 37 degrees Celcius (C) is added thereto at a concentration of generally 30% to 60% (w/v). This is gently mixed to produce desired fusion cells (hybridomas). Then, an appropriate culture medium mentioned above is gradually added to the cells, and this is repeatedly centrifuged to remove the supernatant. Thus, cell fusion agents and such which are unfavorable to hybridoma growth can be removed.

The hybridomas thus obtained can be selected by culture using a conventional selective medium, for example, HAT medium (a culture medium containing hypoxanthine, aminopterin, and thymidine). Cells other than the desired hybridomas (non-fused cells) can be killed by continuing culture in the above HAT medium for a sufficient period of time. Typically, the period is several days to several weeks. Then, hybridomas producing the desired antibody are screened and singly cloned by conventional limiting dilution methods.

The hybridomas thus obtained can be selected using a selection medium based on the selection marker possessed by the myeloma used for cell fusion. For example, HGPRT- or TK-deficient cells can be selected by culture using the HAT medium (a culture medium containing hypoxanthine, aminopterin, and thymidine). Specifically, when HAT-sensitive myeloma cells are used for cell fusion, cells successfully fused with normal cells can selectively proliferate in the HAT medium. Cells other than the desired hybridomas (non-fused cells) can be killed by continuing culture in the above HAT medium for a sufficient period of time. Specifically, desired hybridomas can be selected by culture for generally several days to several weeks. Then, hybridomas producing the desired antibody are screened and singly cloned by conventional limiting dilution methods.

Desired antibodies can be preferably selected and singly cloned by screening methods based on known antigen/antibody reaction. For example, a HER2 S310F-binding monoclonal antibody can bind to HER2 S310F expressed on the cell surface. Such a monoclonal antibody can be screened by fluorescence activated cell sorting (FACS). FACS is a system that assesses the binding of an antibody to cell surface by analyzing cells contacted with a fluorescent antibody using laser beam, and measuring the fluorescence emitted from individual cells.

To screen for hybridomas that produce a monoclonal antibody of the present invention by FACS, HER2 S310F-expressing cells are first prepared. Cells preferably used for screening are mammalian cells in which HER2 S310F is forcedly expressed. As control, the activity of an antibody to bind to cell-surface HER2 S310F can be selectively detected using non-transformed mammalian cells as host cells. Specifically, hybridomas producing an anti-HER2 S310F monoclonal antibody can be isolated by selecting hybridomas that produce an antibody which binds to cells forced to express HER2 S310F, but not to host cells.

Alternatively, the activity of an antibody to bind to immobilized HER2 S310F-expressing cells can be assessed based on the principle of ELISA. For example, HER2 S310F-expressing cells are immobilized to the wells of an ELISA plate. Culture supernatants of hybridomas are contacted with the immobilized cells in the wells, and antibodies that bind to the immobilized cells are detected. When the monoclonal antibodies are derived from mouse, antibodies bound to the cells can be detected using an anti-mouse immunoglobulin antibody. Hybridomas producing a desired antibody having the antigen-binding ability are selected by the above screening, and they can be cloned by a limiting dilution method or the like.

Monoclonal antibody-producing hybridomas thus prepared can be passaged in a conventional culture medium, and stored in liquid nitrogen for a long period.

The above hybridomas are cultured by a conventional method, and desired monoclonal antibodies can be prepared from the culture supernatants. Alternatively, the hybridomas are administered to and grown in compatible mammals, and monoclonal antibodies are prepared from the ascites. The former method is suitable for preparing antibodies with high purity.

Antibodies encoded by antibody genes that are cloned from antibody-producing cells such as the above hybridomas can also be preferably used. A cloned antibody gene is inserted into an appropriate vector, and this is introduced into a host to express the antibody encoded by the gene. Methods for isolating antibody genes, inserting the genes into vectors, and transforming host cells have already been established, for example, by Vandamme et al. (Eur. J. Biochem. (1990) 192(3), 767-775). Methods for producing recombinant antibodies are also known as described below.

Preferably, the present invention provides nucleic acids that encode an antigen-binding molecule or a multispecific antigen-binding molecule of the present invention. The present invention also provides a vector into which the nucleic acid encoding the antigen-binding molecule or multispecific antigen-binding molecule is introduced, i.e., a vector comprising the nucleic acid. Furthermore, the present invention provides a cell comprising the nucleic acid or the vector. The present invention also provides a method for producing the antigen-binding molecule or multispecific antigen-binding molecule by culturing the cell. Preferably, the present invention provides a method of preparing the antigen-binding molecule or multispecific antigen-binding molecule, which comprises expressing a nucleic acid encoding the antigen-binding molecule or multispecific antigen-binding molecule in a cell. The present invention further provides antigen-binding molecule or multispecific antigen-binding molecules produced by the method.

For example, a cDNA encoding the variable region (V region) of an anti-HER2 S310F antibody is prepared from hybridoma cells expressing the anti-HER2 S310F antibody. For this purpose, total RNA is first extracted from hybridomas. Methods used for extracting mRNAs from cells include, for example:
the guanidine ultracentrifugation method (Biochemistry (1979) 18(24), 5294-5299), and
the AGPC method (Anal. Biochem. (1987) 162(1), 156-159)

Extracted mRNAs can be purified using the mRNA Purification Kit (GE Healthcare Bioscience) or such. Alternatively, kits for extracting total mRNA directly from cells, such as the QuickPrep mRNA Purification Kit (GE Healthcare Bioscience), are also commercially available. mRNAs can be prepared from hybridomas using such kits. cDNAs encoding the antibody V region can be synthesized from the prepared mRNAs using a reverse transcriptase. cDNAs can be synthesized using the AMV Reverse Transcriptase First-strand cDNA Synthesis Kit (Seikagaku Co.) or such. Furthermore, the SMART RACE cDNA amplification kit (Clontech) and the PCR-based 5′-RACE method (Proc. Natl. Acad. Sci. USA (1988) 85(23), 8998-9002; Nucleic Acids Res. (1989) 17(8), 2919-2932) can be appropriately used to synthesize and amplify cDNAs. In such a cDNA synthesis process, appropriate restriction enzyme sites described below may be introduced into both ends of a cDNA.

The cDNA fragment of interest is purified from the resulting PCR product, and then this is ligated to a vector DNA. A recombinant vector is thus constructed, and introduced into E. coli or such. After colony selection, the desired recombinant vector can be prepared from the colony-forming E. coli. Then, whether the recombinant vector has the cDNA nucleotide sequence of interest is tested by a known method such as the dideoxy nucleotide chain termination method.

The 5′-RACE method which uses primers to amplify the variable region gene is conveniently used for isolating the gene encoding the variable region. First, a 5′-RACE cDNA library is constructed by cDNA synthesis using RNAs extracted from hybridoma cells as a template. A commercially available kit such as the SMART RACE cDNA amplification kit is appropriately used to synthesize the 5′-RACE cDNA library.

The antibody gene is amplified by PCR using the prepared 5′-RACE cDNA library as a template. Primers for amplifying the mouse antibody gene can be designed based on known antibody gene sequences. The nucleotide sequences of the primers vary depending on the immunoglobulin subclass. Therefore, it is preferable that the subclass is determined in advance using a commercially available kit such as the Iso Strip mouse monoclonal antibody isotyping kit (Roche Diagnostics).

Specifically, for example, primers that allow amplification of genes encoding gamma1, gamma2a, gamma2b, and gamma3 heavy chains and kappa and lambda light chains are used to isolate mouse IgG-encoding genes. In general, a primer that anneals to a constant region site close to the variable region is used as a 3′-side primer to amplify an IgG variable region gene. Meanwhile, a primer attached to a 5′ RACE cDNA library construction kit is used as a 5′-side primer.

PCR products thus amplified are used to reshape immunoglobulins composed of a combination of heavy and light chains. A desired antibody can be selected using the HER2 S310F-binding activity of a reshaped immunoglobulin as an indicator. For example, when the objective is to isolate an antibody against HER2 S310F, it is more preferred that the binding of the antibody to HER2 S310F is specific. A HER2 S310F-binding antibody can be screened, for example, by the following steps:
(1) contacting a HER2 S310F-expressing cell with an antibody comprising the V region encoded by a cDNA isolated from a hybridoma;
(2) detecting the binding of the antibody to the HER2 S310F-expressing cell;
(3) selecting an antibody that specifically binds to the HER2 S310F-expressing cell; and preferably
(4) selecting an antibody the shows stronger binding to HER2 S310F-expressing cell than to wild type HER2, or an antibody that binds to the HER2 S310F-expressing cell but does not bind to wild type HER2.

Methods for detecting the binding of an antibody to HER2 S310F-expressing cells are known. Specifically, the binding of an antibody to HER2 S310F-expressing cells can be detected by the above-described techniques such as FACS. Immobilized samples of HER2 S310F-expressing cells are appropriately used to assess the binding activity of an antibody.

Preferred antibody screening methods that use the binding activity as an indicator also include panning methods using phage vectors. Screening methods using phage vectors are advantageous when the antibody genes are isolated from heavy-chain and light-chain subclass libraries from a polyclonal antibody-expressing cell population. Genes encoding the heavy-chain and light-chain variable regions can be linked by an appropriate linker sequence to form a single-chain Fv (scFv). Phages presenting scFv on their surface can be produced by inserting a gene encoding scFv into a phage vector. The phages are contacted with an antigen of interest. Then, a DNA encoding scFv having the binding activity of interest can be isolated by collecting phages bound to the antigen. This process can be repeated as necessary to enrich scFv having a desired binding activity.

After isolation of the cDNA encoding the V region of the anti-HER2 S310F antibody of interest, the cDNA is digested with restriction enzymes that recognize the restriction sites introduced into both ends of the cDNA. Preferred restriction enzymes recognize and cleave a nucleotide sequence that occurs in the nucleotide sequence of the antibody gene at a low frequency. Furthermore, a restriction site for an enzyme that produces a sticky end is preferably introduced into a vector to insert a single-copy digested fragment in the correct orientation. The cDNA encoding the V region of the anti-HER2 S310F antibody is digested as described above, and this is inserted into an appropriate expression vector to construct an antibody expression vector. In this case, if a gene encoding the antibody constant region (C region) and a gene encoding the above V region are fused in-frame, a chimeric antibody is obtained. Herein, “chimeric antibody” means that the origin of the constant region is different from that of the variable region. Thus, in addition to mouse/human heterochimeric antibodies, human/human allochimeric antibodies are included in the chimeric antibodies of the present invention. A chimeric antibody expression vector can be constructed by inserting the above V region gene into an expression vector that already has the constant region. Specifically, for example, a recognition sequence for a restriction enzyme that excises the above V region gene can be appropriately placed on the 5′ side of an expression vector carrying a DNA encoding a desired antibody constant region (C region). A chimeric antibody expression vector is constructed by fusing in frame the two genes digested with the same combination of restriction enzymes.

To produce an anti-HER2 S310F monoclonal antibody, antibody genes are inserted into an expression vector so that the genes are expressed under the control of an expression regulatory region. The expression regulatory region for antibody expression includes, for example, enhancers and promoters. Furthermore, an appropriate signal sequence may be attached to the amino terminus so that the expressed antibody is secreted to the outside of cells. In the Examples described below, a peptide having the amino acid sequence MGWSCIILFLVATATGVHS (SEQ ID NO: 175) is used as a signal sequence. Meanwhile, other appropriate signal sequences may be attached. The expressed polypeptide is cleaved at the carboxyl terminus of the above sequence, and the resulting polypeptide is secreted to the outside of cells as a mature polypeptide. Then, appropriate host cells are transformed with the expression vector, and recombinant cells expressing the anti-HER2 S310F antibody-encoding DNA are obtained.

DNAs encoding the antibody heavy chain (H chain) and light chain (L chain) are separately inserted into different expression vectors to express the antibody gene. An antibody molecule having the H and L chains can be expressed by co-transfecting the same host cell with vectors into which the H-chain and L-chain genes are respectively inserted. Alternatively, host cells can be transformed with a single expression vector into which DNAs encoding the H and L chains are inserted (see WO 94/11523).

There are various known host cell/expression vector combinations for antibody preparation by introducing isolated antibody genes into appropriate hosts. All of these expression systems are applicable to isolation of domains including antibody variable regions of the present invention. Appropriate eukaryotic cells used as host cells include animal cells, plant cells, and fungal cells. Specifically, the animal cells include, for example, the following cells.
(1) mammalian cells: CHO, COS, myeloma, baby hamster kidney (BHK), HeLa, Vero, or such;
(2) amphibian cells: Xenopus oocytes, or such; and
(3) insect cells: sf9, sf21, Tn5, or such.

In addition, as a plant cell, an antibody gene expression system using cells derived from the Nicotiana genus such as Nicotiana tabacum is known. Callus cultured cells can be appropriately used to transform plant cells.

Furthermore, the following cells can be used as fungal cells: yeasts: the Saccharomyces genus such as Saccharomyces cerevisiae, and the Pichia genus such as Pichia pastoris; and filamentous fungi: the Aspergillus genus such as Aspergillus niger.

Furthermore, antibody gene expression systems that utilize prokaryotic cells are also known. For example, when using bacterial cells, E. coli cells, Bacillus subtilis cells, and such can suitably be utilized in the present invention. Expression vectors carrying the antibody genes of interest are introduced into these cells by transfection. The transfected cells are cultured in vitro, and the desired antibody can be prepared from the culture of transformed cells.

In addition to the above-described host cells, transgenic animals can also be used to produce a recombinant antibody. That is, the antibody can be obtained from an animal into which the gene encoding the antibody of interest is introduced. For example, the antibody gene can be constructed as a fusion gene by inserting in frame into a gene that encodes a protein produced specifically in milk. Goat beta-casein or such can be used, for example, as the protein secreted in milk. DNA fragments containing the fused gene inserted with the antibody gene is injected into a goat embryo, and then this embryo is introduced into a female goat. Desired antibodies can be obtained as a protein fused with the milk protein from milk produced by the transgenic goat born from the embryo-recipient goat (or progeny thereof). In addition, to increase the volume of milk containing the desired antibody produced by the transgenic goat, hormones can be administered to the transgenic goat as necessary (Ebert, K. M. et al., Bio/Technology (1994) 12 (7), 699-702).

### Methods for Producing a Humanized Antibody

When an antigen-binding molecule described herein is administered to human, a domain derived from a genetically recombinant antibody that has been artificially modified to reduce the heterologous antigenicity against human and such, can be appropriately used as the domain of the antigen-binding molecule including an antibody variable region. Such genetically recombinant antibodies include, for example, humanized antibodies. These modified antibodies are appropriately produced by known methods. Furthermore, generally, the binding specificity of a certain antibody can be introduced into another antibody by CDR grafting.

Specifically, humanized antibodies prepared by grafting the CDR of a non-human animal antibody such as a mouse antibody to a human antibody and such are known. Common genetic engineering techniques for obtaining humanized antibodies are also known. Specifically, for example, overlap extension PCR is known as a method for grafting a mouse antibody CDR to a human FR. In overlap extension PCR, a nucleotide sequence encoding a mouse antibody CDR to be grafted is added to primers for synthesizing a human antibody FR. Primers are prepared for each of the four FRs. It is generally considered that when grafting a mouse CDR to a human FR, selecting a human FR that has high identity to a mouse FR is advantageous for maintaining the CDR function. That is, it is generally preferable to use a human FR comprising an amino acid sequence which has high identity to the amino acid sequence of the FR adjacent to the mouse CDR to be grafted.

Nucleotide sequences to be ligated are designed so that they will be connected to each other in frame. Human FRs are individually synthesized using the respective primers. As a result, products in which the mouse CDR-encoding DNA is attached to the individual FR-encoding DNAs are obtained. Nucleotide sequences encoding the mouse CDR of each product are designed so that they overlap with each other. Then, complementary strand synthesis reaction is conducted to anneal the overlapping CDR regions of the products synthesized using a human antibody gene as template. Human FRs are ligated via the mouse CDR sequences by this reaction.

The full length V region gene, in which three CDRs and four FRs are ultimately ligated, is amplified using primers that anneal to its 5′- or 3′-end, which are added with suitable restriction enzyme recognition sequences. An expression vector for humanized antibody can be produced by inserting the DNA obtained as described above and a DNA that encodes a human antibody C region into an expression vector so that they will ligate in frame. After the recombinant vector is transfected into a host to establish recombinant cells, the recombinant cells are cultured, and the DNA encoding the humanized antibody is expressed to produce the humanized antibody in the cell culture (see, European Patent Publication No. EP 239400 and International Patent Publication No. WO 1996/002576).

By qualitatively or quantitatively measuring and evaluating the antigen-binding activity of the humanized antibody produced as described above, one can suitably select human antibody FRs that allow CDRs to form a favorable antigen-binding site when ligated through the CDRs. Amino acid residues in FRs may be substituted as necessary, so that the CDRs of a reshaped human antibody form an appropriate antigen-binding site. For example, amino acid sequence mutations can be introduced into FRs by applying the PCR method used for grafting a mouse CDR into a human FR. More specifically, partial nucleotide sequence mutations can be introduced into primers that anneal to the FR. Nucleotide sequence mutations are introduced into the FRs synthesized by using such primers. Mutant FR sequences having the desired characteristics can be selected by measuring and evaluating the activity of the amino acid-substituted mutant antibody to bind to the antigen by the above-mentioned method (Sato, K. et al., Cancer Res. (1993) 53: 851-856) Methods for producing a human antibody.

Alternatively, desired human antibodies can be obtained by immunizing transgenic animals having the entire repertoire of human antibody genes (see WO 1993/012227; WO 1992/003918; WO 1994/002602; WO 1994/025585; WO 1996/034096; WO 1996/033735) by DNA immunization.

Furthermore, techniques for preparing human antibodies by panning using human antibody libraries are also known. For example, the V region of a human antibody is expressed as a single-chain antibody (scFv) on phage surface by the phage display method. Phages expressing a scFv that binds to the antigen can be selected. The DNA sequence encoding the human antibody V region that binds to the antigen can be determined by analyzing the genes of selected phages. The DNA sequence of the scFv that binds to the antigen is determined. An expression vector is prepared by fusing the V region sequence in frame with the C region sequence of a desired human antibody, and inserting this into an appropriate expression vector. The expression vector is introduced into cells appropriate for expression such as those described above. The human antibody can be produced by expressing the human antibody-encoding gene in the cells. These methods are already known (see WO 1992/001047; WO 1992/020791; WO 1993/006213; WO 1993/011236; WO 1993/019172; WO 1995/001438; WO 1995/015388).

### Vector

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

### Host Cell

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

### Epitope

“Epitope” means an antigenic determinant in an antigen, and refers to an antigen site to which the antigen-binding domain of an antigen-binding molecule or antibody disclosed herein binds. Thus, for example, the epitope can be defined according to its structure. Alternatively, the epitope may be defined according to the antigen-binding activity of an antigen-binding molecule or antibody that recognizes the epitope. When the antigen is a peptide or polypeptide, the epitope can be specified by the amino acid residues forming the epitope. Alternatively, when the epitope is a sugar chain, the epitope can be specified by its specific sugar chain structure.

A linear epitope is an epitope that contains an epitope whose primary amino acid sequence is recognized. Such a linear epitope typically contains at least three and most commonly at least five, for example, about 8 to 10 or 6 to 20 amino acids in its specific sequence.

In contrast to the linear epitope, “conformational epitope” is an epitope in which the primary amino acid sequence containing the epitope is not the only determinant of the recognized epitope (for example, the primary amino acid sequence of a conformational epitope is not necessarily recognized by an epitope-defining antibody). Conformational epitopes may contain a greater number of amino acids compared to linear epitopes. A conformational epitope-recognizing antigen-binding domain recognizes the three-dimensional structure of a peptide or protein. For example, when a protein molecule folds and forms a three-dimensional structure, amino acids and/or polypeptide main chains that form a conformational epitope become aligned, and the epitope is made recognizable by the antigen-binding domain. Methods for determining epitope conformations include, for example, X ray crystallography, two-dimensional nuclear magnetic resonance, site-specific spin labeling, and electron paramagnetic resonance, but are not limited thereto. See, for example, Epitope Mapping Protocols in Methods in Molecular Biology (1996), Vol. 66, Morris (ed.).

Examples of a method for assessing the epitope binding by a test antigen-binding molecule or antibody containing an anti-HER2 S310F antigen-binding domain are described below. According to the examples below, methods for assessing the epitope binding by a test antigen-binding molecule or antibody containing an antigen-binding domain for an antigen other than HER2 S310F, can also be appropriately conducted.

For example, whether a test antigen-binding molecule or antibody containing an anti-HER2 S310F antigen-binding domain recognizes a linear epitope in the HER2 S310F molecule can be confirmed for example as mentioned below. A linear peptide comprising an amino acid sequence forming the extracellular domain of HER2 S310F is synthesized for the above purpose. The peptide can be synthesized chemically, or obtained by genetic engineering techniques using a region encoding the amino acid sequence corresponding to the extracellular domain in a HER2 S310F cDNA. Then, a test antigen-binding molecule or antibody containing an anti-HER2 S310F antigen-binding domain is assessed for its binding activity towards a linear peptide comprising the amino acid sequence forming the extracellular domain. For example, an immobilized linear peptide can be used as an antigen by ELISA to evaluate the binding activity of the polypeptide complex towards the peptide. Alternatively, the binding activity towards a linear peptide can be assessed based on the level that the linear peptide inhibits the binding of the antigen-binding molecule or antibody to HER2 S310F-expressing cells. These tests can demonstrate the binding activity of the antigen-binding molecule or antibody towards the linear peptide.

Whether a test antigen-binding molecule or antibody containing an anti-HER2 S310F antigen-binding domain recognizes a conformational epitope can be assessed as follows. HER2 S310F-expressing cells are prepared for the above purpose. A test antigen-binding molecule or antibody containing an anti-HER2 S310F antigen-binding domain can be determined to recognize a conformational epitope when it strongly binds to HER2 S310F-expressing cells upon contact, but does not substantially bind to an immobilized linear peptide comprising an amino acid sequence forming the extracellular domain of HER2 S310F. Herein, “not substantially bind” means that the binding activity is 80% or less, generally 50% or less, preferably 30% or less, and particularly preferably 15% or less compared to the binding activity towards cells expressing HER2 S310F.

Methods for assaying the binding activity of a test antigen-binding molecule or antibody containing an anti-HER2 S310F antigen-binding domain towards HER2 S310F-expressing cells include, for example, the methods described in Antibodies: A Laboratory Manual (Ed Harlow, David Lane, Cold Spring Harbor Laboratory (1988) 359-420). Specifically, the assessment can be performed based on the principle of ELISA or fluorescence activated cell sorting (FACS) using HER2 S310F-expressing cells as antigen.

In the ELISA format, the binding activity of a test antigen-binding molecule or antibody containing an anti-HER2 S310F antigen-binding domain towards HER2 S310F-expressing cells can be assessed quantitatively by comparing the levels of signal generated by enzymatic reaction. Specifically, a test polypeptide complex is added to an ELISA plate onto which HER2 S310F-expressing cells are immobilized. Then, the test antigen-binding molecule or antibody bound to the cells is detected using an enzyme-labeled antibody that recognizes the test antigen-binding molecule or antibody. Alternatively, when FACS is used, a dilution series of a test antigen-binding molecule or antibody is prepared, and the antibody binding titer for HER2 S310F-expressing cells can be determined to compare the binding activity of the test antigen-binding molecule or antibody towards HER2 S310F-expressing cells.

The binding of a test antigen-binding molecule or antibody towards an antigen expressed on the surface of cells suspended in buffer or the like can be detected using a flow cytometer. Known flow cytometers include, for example, the following devices:
FACSCanto®II
FACSAria®
FACSArray®
FACSVantage®SE
FACSCalibur® (all are trade names of BD Biosciences)
EPICS ALTRA HyPerSort
Cytomics FC 500
EPICS XL-MCL ADC EPICS XL ADC
Cell Lab Quanta/Cell Lab Quanta SC (all are trade names of Beckman Coulter)

Preferable methods for assaying the binding activity of a test antigen-binding molecule or antibody containing an anti-HER2 S310F antigen-binding domain towards an antigen include, for example, the following method. First, HER2 S310F-expressing cells are reacted with a test antigen-binding molecule or antibody, and then this is stained with an FITC-labeled secondary antibody that recognizes the antigen-binding molecule or antibody. The test antigen-binding molecule or antibody is appropriately diluted with a suitable buffer to prepare the antigen-binding molecule or antibody at a desired concentration. For example, the antigen-binding molecule or antibody can be used at a concentration within the range of 10 micrograms (micro g)/ml to 10 ng/ml. Then, the fluorescence intensity and cell count are determined using FACSCalibur (BD). The fluorescence intensity obtained by analysis using the CELL QUEST Software (BD), i.e., the Geometric Mean value, reflects the quantity of antibody bound to cells. That is, the binding activity of a test antigen-binding molecule or antibody, which is represented by the quantity of the test antigen-binding molecule or antibody bound, can be determined by measuring the Geometric Mean (Geo-mean) value.

Whether a test antigen-binding molecule or antibody containing an anti-HER2 S310F antigen-binding domain shares a common epitope with another antigen-binding molecule or antibody can be assessed based on the competition between the two antigen-binding molecules or antibodies for the same epitope. The competition between the antigen-binding molecules or antibodies can be detected by cross-blocking assay or the like. For example, the competitive ELISA assay is a preferred cross-blocking assay.

Specifically, in cross-blocking assay, the HER2 S310F protein immobilized to the wells of a microtiter plate is pre-incubated in the presence or absence of a candidate competitor antigen-binding molecule or antibody, and then a test antigen-binding molecule or antibody is added thereto. The quantity of test antigen-binding molecule or antibody bound to the HER2 S310F protein in the wells is indirectly correlated with the binding ability of a candidate competitor antigen-binding molecule or antibody that competes for the binding to the same epitope. That is, the greater the affinity of the competitor antigen-binding molecule or antibody for the same epitope, the lower the binding activity of the test antigen-binding molecule or antibody towards the HER2 S310F protein-coated wells.

The quantity of the test antigen-binding molecule or antibody bound to the wells via the HER2 S310F protein can be readily determined by labeling the antigen-binding molecule or antibody in advance. For example, a biotin-labeled antigen-binding molecule or antibody is measured using an avidin/peroxidase conjugate and appropriate substrate. In particular, cross-blocking assay that uses enzyme labels such as peroxidase is called “competitive ELISA assay”. The antigen-binding molecule or antibody can also be labeled with other labeling substances that enable detection or measurement. Specifically, radiolabels, fluorescent labels, and such are known.

When the candidate competitor antigen-binding molecule or antibody can block the binding by a test antigen-binding molecule or antibody containing an anti-HER2 S310F antigen-binding domain by at least 20%, preferably at least 20 to 50%, and more preferably at least 50% compared to the binding activity in a control experiment conducted in the absence of the competitor antigen-binding molecule or antibody, the test antigen-binding molecule or antibody is determined to substantially bind to the same epitope bound by the competitor antigen-binding molecule or antibody, or compete for the binding to the same epitope.

When the structure of an epitope bound by a test antigen-binding molecule or antibody containing an anti-HER2 S310F antigen-binding domain has already been identified, whether the test and control antigen-binding molecules or antibodies share a common epitope can be assessed by comparing the binding activities of the two antigen-binding molecules or antibodies towards a peptide prepared by introducing amino acid mutations into the peptide forming the epitope.

To measure the above binding activities, for example, the binding activities of test and control antigen-binding molecules or antibodies towards a linear peptide into which a mutation is introduced are compared in the above ELISA format. Besides the ELISA methods, the binding activity towards the mutant peptide bound to a column can be determined by flowing test and control antigen-binding molecules or antibodies in the column, and then quantifying the antigen-binding molecule or antibody eluted in the elution solution. Methods for adsorbing a mutant peptide to a column, for example, in the form of a GST fusion peptide, are known.

Alternatively, when the identified epitope is a conformational epitope, whether test and control antigen-binding molecules or antibodies share a common epitope can be assessed by the following method. First, HER2 S310F-expressing cells and cells expressing HER2 S310F with a mutation introduced into the epitope are prepared. The test and control antigen-binding molecules or antibodies are added to a cell suspension prepared by suspending these cells in an appropriate buffer such as PBS. Then, the cell suspensions are appropriately washed with a buffer, and an FITC-labeled antibody that recognizes the test and control antigen-binding molecules or antibodies is added thereto. The fluorescence intensity and number of cells stained with the labeled antibody are determined using FACSCalibur (BD). The test and control antigen-binding molecules or antibodies are appropriately diluted using a suitable buffer, and used at desired concentrations. For example, they may be used at a concentration within the range of 10 micro g/ml to 10 ng/ml. The fluorescence intensity determined by analysis using the CELL QUEST Software (BD), i.e., the Geometric Mean value, reflects the quantity of labeled antibody bound to cells. That is, the binding activities of the test and control antigen-binding molecules or antibodies, which are represented by the quantity of labeled antibody bound, can be determined by measuring the Geometric Mean value.

In the above method, whether an antigen-binding molecule or antibody does “not substantially bind to cells expressing mutant HER2 S310F” can be assessed, for example, by the following method. First, the test and control antigen-binding molecules or antibodies bound to cells expressing mutant HER2 S310F are stained with a labeled antibody. Then, the fluorescence intensity of the cells is determined. When FACSCalibur is used for fluorescence detection by flow cytometry, the determined fluorescence intensity can be analyzed using the CELL QUEST Software. From the Geometric Mean values in the presence and absence of the antigen-binding molecule or antibody, the comparison value (delta Geo-Mean) can be calculated according to the following formula to determine the ratio of increase in fluorescence intensity as a result of the binding by the antigen-binding molecule or antibody.

delta Geo-Mean=Geo-Mean (in the presence of the antigen-binding molecule or antibody)/Geo-Mean (in the absence of the antigen-binding molecule or antibody)

The Geometric Mean comparison value (delta Geo-Mean value for the mutant HER2 S310F molecule) determined by the above analysis, which reflects the quantity of a test antigen-binding molecule or antibody bound to cells expressing mutant HER2 S310F, is compared to the delta Geo-Mean comparison value that reflects the quantity of the test antigen-binding molecule or antibody bound to HER2 S310F-expressing cells. In this case, the concentrations of the test antigen-binding molecule or antibody used to determine the delta Geo-Mean comparison values for HER2 S310F-expressing cells and cells expressing mutant HER2 S310F are particularly preferably adjusted to be equal or substantially equal. An antigen-binding molecule or antibody that has been confirmed to recognize an epitope in HER2 S310F is used as a control antigen-binding molecule or antibody.

If the delta Geo-Mean comparison value of a test antigen-binding molecule or antibody for cells expressing mutant HER2 S310F is smaller than the delta Geo-Mean comparison value of the test antigen-binding molecule or antibody for HER2 S310F-expressing cells by at least 80%, preferably 50%, more preferably 30%, and particularly preferably 15%, then the test antigen-binding molecule or antibody “does not substantially bind to cells expressing mutant HER2 S310F”. The formula for determining the Geo-Mean (Geometric Mean) value is described in the CELL QUEST Software User's Guide (BD biosciences). When the comparison shows that the comparison values are substantially equivalent, the epitope for the test and control antigen-binding molecules or antibodies can be determined to be the same.

### Specificity

“Specific” means that a molecule that binds specifically to one or more binding partners does not show any significant binding to molecules other than the partners. Furthermore, “specific” is also used when an antigen-binding domain is specific to a particular epitope of multiple epitopes contained in an antigen. When an epitope bound by an antigen-binding domain is contained in multiple different antigens, an antigen-binding molecule containing the antigen-binding domain can bind to various antigens that have the epitope.

In the context of this specification, preferably, an antibody of the present invention is “specific” to the HER2 S310F mutant with low or no cross-reactivity against wild-type HER2. The high specificity of the antibody of the present invention may be demonstrated by a smaller KD ratio of the KD value for binding to HER2 S310F to the KD value for binding to wild type HER2 (i.e. KDHER2 S310F/KDwild type HER2), compared to the KD ratio of a control antibody which is not “specific” to HER2 S310F. Wild-type HER2 is expressed in normal tissues, and involved in various cellular processes such as proliferation, differentiation, cell migration, and cell survival. Therefore, the possible concern is that a non-specific HER2 antibody which targets wild-type HER2 (instead of or in addition to HER2 S310F) could cause serious cell toxicity and damages to healthy tissues or organs. The specificity towards the HER2 S310F mutant is particularly desirable in that the risk of adverse or side effects can be significantly reduced, and higher safety of therapeutic agents containing such a “specific” antibody can be expected.

### Monospecific Antigen-Binding Molecules

The term “monospecific antigen-binding molecule” is used to refer to antigen-binding molecules that specifically bind to only one type of antigen. A favorable example of a monospecific antigen-binding molecule is an antigen-binding molecule that comprises a single type of antigen-binding domain. Monospecific antigen-binding molecules can comprise a single antigen-binding domain or a plurality of antigen-binding domains of the same type. A favorable example of monospecific antigen-binding molecules is a monospecific antibody. When the monospecific antigen-binding molecule is a monospecific antibody of the IgG form, the monospecific antibody comprises two antibody variable fragments that have the same antigen-binding specificity.

In the context of this specification, preferably, a monospecific antigen-binding molecules or monospecific antibody of the present invention has a single antigen-binding domain or two or more of antigen-binding domains that specifically bind to a human HER2 S310F mutant but do not (specifically) bind to wild-type HER2.

### Antibody Fragment

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g. scFv); and multispecific antibodies formed from antibody fragments.

The terms “full length antibody,” “intact antibody,” and “whole antibody” are used herein interchangeably to refer to an antibody having a structure substantially similar to a native antibody structure or having heavy chains that contain an Fc region as defined herein.

### Variable Fragment (Fv)

Herein, the term “variable fragment (Fv)” refers to the minimum unit of an antibody-derived antigen-binding domain that is composed of a pair of the antibody light chain variable region (VL) and antibody heavy chain variable region (VH). In 1988, Skerra and Pluckthun found that homogeneous and active antibodies can be prepared from the E. coli periplasm fraction by inserting an antibody gene downstream of a bacterial signal sequence and inducing expression of the gene in E. coli (Science (1988) 240(4855), 1038-1041). In the Fv prepared from the periplasm fraction, VH associates with VL in a manner so as to bind to an antigen. scFv, single-chain antibody, and sc(Fv)2

Herein, the terms “scFv”, “single-chain antibody”, and “sc(Fv)2” all refer to an antibody fragment of a single polypeptide chain that contains variable regions derived from the heavy and light chains, but not the constant region. In general, a single-chain antibody also contains a polypeptide linker between the VH and VL domains, which enables formation of a desired structure that is thought to allow antigen binding. The single-chain antibody is discussed in detail by Pluckthun in “The Pharmacology of Monoclonal Antibodies, Vol. 113, Rosenburg and Moore, eds., Springer-Verlag, New York, 269-315 (1994)”. See also International Patent Publication WO 1988/001649; U.S. Pat. Nos. 4,946,778 and 5,260,203. In a particular embodiment, the single-chain antibody can be bispecific and/or humanized.

scFv is an antigen-binding domain in which VH and VL forming Fv are linked together by a peptide linker (Proc. Natl. Acad. Sci. U.S.A. (1988) 85(16), 5879-5883). VH and VL can be retained in close proximity by the peptide linker.

sc(Fv)2 is a single-chain antibody in which four variable regions of two VL and two VH are linked by linkers such as peptide linkers to form a single chain (J Immunol. Methods (1999) 231(1-2), 177-189). The two VH and two VL may be derived from different monoclonal antibodies. Such sc(Fv)2 preferably includes, for example, a bispecific sc(Fv)2 that recognizes two epitopes present in a single antigen as disclosed in the Journal of Immunology (1994) 152(11), 5368-5374. sc(Fv)2 can be produced by methods known to those skilled in the art. For example, sc(Fv)2 can be produced by linking scFv by a linker such as a peptide linker.

Herein, the form of an antigen-binding domain forming an sc(Fv)2 include an antibody in which the two VH units and two VL units are arranged in the order of VH, VL, VH, and VL ([VH]-linker-[VL]-linker-[VH]-linker-[VL]) beginning from the N terminus of a single-chain polypeptide. The order of the two VH units and two VL units is not limited to the above form, and they may be arranged in any order. Examples of the form are listed below.
[VL]-linker-[VH]-linker-[VH]-linker-[VL][VH]-linker-[VL]-linker-[VL]-linker-[VH][VH]-linker-[VH]-linker-[VL]-linker-[VL][VL]-linker-[VL]-linker-[VH]-linker-[VH][VL]-linker-[VH]-linker-[VL]-linker-[VH]

The molecular form of sc(Fv)2 is also described in detail in WO 2006/132352. According to these descriptions, those skilled in the art can appropriately prepare desired sc(Fv)2 to produce the polypeptide complexes disclosed herein.

Furthermore, the antigen-binding molecules or antibodies of the present invention may be conjugated with a carrier polymer such as PEG or an organic compound such as an anticancer agent. Alternatively, a sugar chain addition sequence is preferably inserted into the antigen-binding molecules or antibodies such that the sugar chain produces a desired effect.

The linkers to be used for linking the variable regions of an antibody comprise arbitrary peptide linkers that can be introduced by genetic engineering, synthetic linkers, and linkers disclosed in, for example, Protein Engineering, 9(3), 299-305, 1996. However, peptide linkers are preferred in the present invention. The length of the peptide linkers is not particularly limited, and can be suitably selected by those skilled in the art according to the purpose. The length is preferably five amino acids or more (without particular limitation, the upper limit is generally 30 amino acids or less, preferably 20 amino acids or less), and particularly preferably 15 amino acids. When sc(Fv)2 contains three peptide linkers, their length may be all the same or different.

For example, such peptide linkers include:

Ser
Gly Ser
Gly Gly Ser
Ser Gly Gly
Gly Gly Gly Ser (SEQ ID NO: 167)
Ser Gly Gly Gly (SEQ ID NO: 168)
Gly Gly Gly Gly Ser (SEQ ID NO: 169)
Ser Gly Gly Gly Gly (SEQ ID NO: 170)
Gly Gly Gly Gly Gly Ser (SEQ ID NO: 171)
Ser Gly Gly Gly Gly Gly (SEQ ID NO: 172)
Gly Gly Gly Gly Gly Gly Ser (SEQ ID NO: 173)
Ser Gly Gly Gly Gly Gly Gly (SEQ ID NO: 174)
(Gly Gly Gly Gly Ser (SEQ ID NO: 169))n
(Ser Gly Gly Gly Gly (SEQ ID NO: 170))n

where n is an integer of 1 or larger. The length or sequences of peptide linkers can be selected accordingly by those skilled in the art depending on the purpose.

Synthetic linkers (chemical crosslinking agents) are routinely used to crosslink peptides, and examples include:
N-hydroxy succinimide (NHS),
disuccinimidyl suberate (DSS),
bis(sulfosuccinimidyl) suberate (BS3),
dithiobis(succinimidyl propionate) (DSP),
dithiobis(sulfosuccinimidyl propionate) (DTSSP),
ethylene glycol bis(succinimidyl succinate) (EGS),
ethylene glycol bis(sulfosuccinimidyl succinate) (sulfo-EGS),
disuccinimidyl tartrate (DST), disulfosuccinimidyl tartrate (sulfo-DST),
bis[2-(succinimidoxycarbonyloxy)ethyl]sulfone (BSOCOES), and
bis[2-(sulfosuccinimidoxycarbonyloxy)ethyl]sulfone (sulfo-BSOCOES).

These crosslinking agents are commercially available.

In general, three linkers are required to link four antibody variable regions together. The linkers to be used may be of the same type or different types. Fab, F(ab′)2, and Fab′

“Fab” consists of a single light chain, and a CH1 domain and variable region from a single heavy chain. The heavy chain of Fab molecule cannot form disulfide bonds with another heavy chain molecule.

“F(ab′)2” or “Fab” is produced by treating an immunoglobulin (monoclonal antibody) with a protease such as pepsin and papain, and refers to an antibody fragment generated by digesting an immunoglobulin (monoclonal antibody) near the disulfide bonds present between the hinge regions in each of the two H chains. For example, papain cleaves IgG upstream of the disulfide bonds present between the hinge regions in each of the two H chains to generate two homologous antibody fragments, in which an L chain comprising VL (L-chain variable region) and CL (L-chain constant region) is linked to an H-chain fragment comprising VH (H-chain variable region) and CH gamma 1 (gamma 1 region in an H-chain constant region) via a disulfide bond at their C-terminal regions. Each of these two homologous antibody fragments is called Fab′.

“F(ab′)2” consists of two light chains and two heavy chains comprising the constant region of a CH1 domain and a portion of CH2 domains so that disulfide bonds are formed between the two heavy chains. The F(ab′)2 disclosed herein can be preferably produced as follows. A whole monoclonal antibody or such comprising a desired antigen-binding domain is partially digested with a protease such as pepsin; and Fc fragments are removed by adsorption onto a Protein A column. The protease is not particularly limited, as long as it can cleave the whole antibody in a selective manner to produce F(ab′)2 under an appropriate setup enzyme reaction condition such as pH. Such proteases include, for example, pepsin and ficin.

### Fc Region

The term “Fe region” or “Fc domain” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) or glycine-lysine (residues 446-447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

### Fc Receptor

The term “Fe receptor” or “FcR” refers to a receptor that binds to the Fc region of an antibody. In some embodiments, an FcR is a native human FcR. In some embodiments, an FcR is one which binds an IgG antibody (a gamma receptor) and includes receptors of the Fc gamma RI, Fc gamma RII, and Fc gamma RIII subclasses, including allelic variants and alternatively spliced forms of those receptors. Fc gamma RII receptors include Fc gamma RIIA (an “activating receptor”) and Fc gamma RIIB (an “inhibiting receptor”), which have similar amino acid sequences that differ primarily in the cytoplasmic domains thereof. Activating receptor Fc gamma RIIA contains an immunoreceptor tyrosine-based activation motif (ITAM) in its cytoplasmic domain. Inhibiting receptor Fc gamma RIIB contains an immunoreceptor tyrosine-based inhibition motif (ITIM) in its cytoplasmic domain. (see, e.g., Daeron, Annu. Rev. Immunol. 15:203-234 (1997)). FcRs are reviewed, for example, in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein.

The term “Fe receptor” or “FcR” also includes the neonatal receptor, FcRn, which is responsible for the transfer of maternal IgGs to the fetus (Guyer et al., J. Immunol. 117:587 (1976) and Kim et al., J. Immunol. 24:249 (1994)) and regulation of homeostasis of immunoglobulins. Methods of measuring binding to FcRn are known (see, e.g., Ghetie and Ward., Immunol. Today 18(12):592-598 (1997); Ghetie et al., Nature Biotechnology, 15(7):637-640 (1997); Hinton et al., J. Biol. Chem. 279(8):6213-6216 (2004); WO 2004/92219 (Hinton et al.).

Binding to human FcRn in vivo and plasma half life of human FcRn high affinity binding polypeptides can be assayed, e.g., in transgenic mice or transfected human cell lines expressing human FcRn, or in primates to which the polypeptides with a variant Fc region are administered. WO 2000/42072 (Presta) describes antibody variants with increased or decreased binding to FcRs. See also, e.g., Shields et al. J. Biol. Chem. 9(2):6591-6604 (2001).

### Fc Gamma Receptor

Fc gamma receptor refers to a receptor capable of binding to the Fc domain of monoclonal IgG1, IgG2, IgG3, or IgG4 antibodies, and includes all members belonging to the family of proteins substantially encoded by an Fc gamma receptor gene. In human, the family includes Fc gamma RI (CD64) including isoforms Fc gamma RIa, Fc gamma RIb and Fc gamma RIc; Fc gamma RII (CD32) including isoforms Fc gamma RIIa (including allotype H131 and R131), Fc gamma RIIb (including Fc gamma RIIb-1 and Fc gamma RIIb-2), and Fc gamma RIIc; and Fc gamma RIII (CD16) including isoform Fc gamma RIIIa (including allotype V158 and F158) and Fc gamma RIIIb (including allotype Fc gamma RIIIb-NA1 and Fc gamma RIIIb-NA2); as well as all unidentified human Fc gamma receptors, Fc gamma receptor isoforms, and allotypes thereof. However, Fc gamma receptor is not limited to these examples. Without being limited thereto, Fc gamma receptor includes those derived from humans, mice, rats, rabbits, and monkeys. Fc gamma receptor may be derived from any organisms. Mouse Fc gamma receptor includes, without being limited to, Fc gamma RI (CD64), Fc gamma RII (CD32), Fc gamma RIII (CD16), and Fc gamma RIII-2 (CD16-2), as well as all unidentified mouse Fc gamma receptors, Fc gamma receptor isoforms, and allotypes thereof. Such preferred Fc gamma receptors include, for example, human Fc gamma RI (CD64), Fc gamma RIIA (CD32), Fc gamma RIIB (CD32), Fc gamma RIIIA (CD16), and/or Fc gamma RIIIB (CD16). The polynucleotide sequence and amino acid sequence of Fc gamma RI are shown in SEQ ID NOs: 157 (NM_000566.3) and 152 (NP_000557.1), respectively; the polynucleotide sequence and amino acid sequence of Fc gamma RIIA are shown in SEQ ID NOs: 158 (BC020823.1) and 153 (AAH20823.1), respectively; the polynucleotide sequence and amino acid sequence of Fc gamma RIIB are shown in SEQ ID NOs: 159 (BC146678.1) and 154 (AAI46679.1), respectively; the polynucleotide sequence and amino acid sequence of Fc gamma RIIIA are shown in SEQ ID NOs: 160 (BC033678.1) and 155 (AAH333678.1), respectively; and the polynucleotide sequence and amino acid sequence of Fc gamma RIIIB are shown in SEQ ID NOs: 161 (BC128562.1) and 156 (AAI28563.1), respectively (RefSeq accession number is shown in each parentheses). Whether an Fc gamma receptor has binding activity to the Fc domain of a monoclonal IgG1, IgG2, IgG3, or IgG4 antibody can be assessed by ALPHA screen (Amplified Luminescent Proximity Homogeneous Assay), surface plasmon resonance (SPR)-based BIACORE method, and others (Proc. Natl. Acad. Sci. USA (2006) 103(11), 4005-4010), in addition to the above-described FACS and ELISA formats.

Meanwhile, “Fc ligand” or “effector ligand” refers to a molecule and preferably a polypeptide that binds to an antibody Fc domain, forming an Fc/Fc ligand complex. The molecule may be derived from any organisms. The binding of an Fc ligand to Fc preferably induces one or more effector functions. Such Fc ligands include, but are not limited to, Fc receptors, Fc gamma receptor, Fc alpha receptor, Fc beta receptor, FcRn, C1q, and C3, mannan-binding lectin, mannose receptor, Staphylococcus Protein A, Staphylococcus Protein G, and viral Fc gamma receptors. The Fc ligands also include Fc receptor homologs (FcRH) (Davis et al., (2002) Immunological Reviews 190, 123-136), which are a family of Fc receptors homologous to Fc gamma receptor. The Fc ligands also include unidentified molecules that bind to Fc.

### Fc Gamma Receptor-Binding Activity

The impaired binding activity of Fc domain to any of the Fc gamma receptors Fc gamma RI, Fc gamma RIIA, Fc gamma RIIB, Fc gamma RIIIA, and/or Fc gamma RIIIB can be assessed by using the above-described FACS and ELISA formats as well as ALPHA screen (Amplified Luminescent Proximity Homogeneous Assay) and surface plasmon resonance (SPR)-based BIACORE method (Proc. Natl. Acad. Sci. USA (2006) 103(11), 4005-4010).

ALPHA screen is performed by the ALPHA technology based on the principle described below using two types of beads: donor and acceptor beads. A luminescent signal is detected only when molecules linked to the donor beads interact biologically with molecules linked to the acceptor beads and when the two beads are located in close proximity. Excited by laser beam, the photosensitizer in a donor bead converts oxygen around the bead into excited singlet oxygen. When the singlet oxygen diffuses around the donor beads and reaches the acceptor beads located in close proximity, a chemiluminescent reaction within the acceptor beads is induced. This reaction ultimately results in light emission. If molecules linked to the donor beads do not interact with molecules linked to the acceptor beads, the singlet oxygen produced by donor beads do not reach the acceptor beads and chemiluminescent reaction does not occur.

For example, a biotin-labeled antigen-binding molecule or antibody is immobilized to the donor beads and glutathione S-transferase (GST)-tagged Fc gamma receptor is immobilized to the acceptor beads. In the absence of an antigen-binding molecule or antibody comprising a competitive mutant Fc domain, Fc gamma receptor interacts with an antigen-binding molecule or antibody comprising a wild-type Fc domain, inducing a signal of 520 to 620 nm as a result. The antigen-binding molecule or antibody having a non-tagged mutant Fc domain competes with the antigen-binding molecule or antibody comprising a wild-type Fc domain for the interaction with Fc gamma receptor. The relative binding affinity can be determined by quantifying the reduction of fluorescence as a result of competition. Methods for biotinylating the antigen-binding molecules or antibodies such as antibodies using Sulfo-NHS-biotin or the like are known. Appropriate methods for adding the GST tag to an Fc gamma receptor include methods that involve fusing polypeptides encoding Fc gamma receptor and GST in-frame, expressing the fused gene using cells introduced with a vector carrying the gene, and then purifying using a glutathione column. The induced signal can be preferably analyzed, for example, by fitting to a one-site competition model based on nonlinear regression analysis using software such as GRAPHPAD PRISM (GraphPad; San Diego).

One of the substances for observing their interaction is immobilized as a ligand onto the gold thin layer of a sensor chip. When light is shed on the rear surface of the sensor chip so that total reflection occurs at the interface between the gold thin layer and glass, the intensity of reflected light is partially reduced at a certain site (SPR signal). The other substance for observing their interaction is injected as an analyte onto the surface of the sensor chip. The mass of immobilized ligand molecule increases when the analyte binds to the ligand. This alters the refraction index of solvent on the surface of the sensor chip. The change in refraction index causes a positional shift of SPR signal (conversely, the dissociation shifts the signal back to the original position). In the Biacore system, the amount of shift described above (i.e., the change of mass on the sensor chip surface) is plotted on the vertical axis, and thus the change of mass over time is shown as measured data (sensorgram). Kinetic parameters (association rate constant (ka) and dissociation rate constant (kd)) are determined from the curve of sensorgram, and affinity (KD) is determined from the ratio between these two constants. Inhibition assay is preferably used in the BIACORE methods. Examples of such inhibition assay are described in Proc. Natl. Acad. Sci. USA (2006) 103(11), 4005-4010.

Fc Region with a Reduced Fc Gamma Receptor-Binding Activity

Herein, “a reduced Fc gamma receptor-binding activity” means, for example, that based on the above-described analysis method the competitive activity of a test antigen-binding molecule or antibody is 50% or less, preferably 45% or less, 40% or less, 35% or less, 30% or less, 20% or less, or 15% or less, and particularly preferably 10% or less, 9% or less, 8% or less, 7% or less, 6% or less, 5% or less, 4% or less, 3% or less, 2% or less, or 1% or less than the competitive activity of a control (e.g., wild-type) antigen-binding molecule or antibody. In an embodiment, the “reduced Fc gamma receptor-binding activity” is a reduced Fc gamma receptor-binding activity compared to a wild type Fc domain of IgG1.

Antigen-binding molecules or antibodies comprising the Fc domain of a monoclonal IgG1, IgG2, IgG3, or IgG4 antibody can be appropriately used as control antigen-binding molecules or antibodies. The Fc domain structures are shown in SEQ ID NOs: 148 (A is added to the N terminus of RefSeq accession number AAC82527.1), 149 (A is added to the N terminus of RefSeq accession number AAB59393.1), 150 (A is added to the N terminus of RefSeq accession number CAA27268.1), and 151 (A is added to the N terminus of RefSeq accession number AAB59394.1). Furthermore, when an antigen-binding molecule or antibody comprising an Fc domain mutant of an antibody of a particular isotype is used as a test substance, the effect of the mutation of the mutant on the Fc gamma receptor-binding activity is assessed using as a control an antigen-binding molecule or antibody comprising an Fc domain of the same isotype. As described above, antigen-binding molecules or antibodies comprising an Fc domain mutant whose Fc gamma receptor-binding activity has been judged to be reduced are appropriately prepared.

Such known mutants include, for example, mutants having a deletion of amino acids 231A-238S (EU numbering) (WO 2009/011941), as well as mutants C226S, C229S, P238S, (C220S) (J. Rheumatol (2007) 34, 11); C226S and C229S (Hum. Antibod. Hybridomas (1990) 1(1), 47-54); C226S, C229S, E233P, L234V, and L235A (Blood (2007) 109, 1185-1192).

Specifically, the preferred antigen-binding molecules or antibodies include those comprising an Fc domain with a mutation (such as substitution) of at least one amino acid selected from the following amino acid positions: 220, 226, 229, 231, 232, 233, 234, 235, 236, 237, 238, 239, 240, 264, 265, 266, 267, 269, 270, 295, 296, 297, 298, 299, 300, 325, 327, 328, 329, 330, 331, or 332 (EU numbering), in the amino acids forming the Fc domain of an antibody of a particular isotype. The isotype of antibody from which the Fc domain originates is not particularly limited, and it is possible to use an appropriate Fc domain derived from a monoclonal IgG1, IgG2, IgG3, or IgG4 antibody. It is preferable to use Fc domains derived from IgG1 antibodies.

The preferred antigen-binding molecules or antibodies include, for example, those comprising an Fc domain which has any one of the substitutions shown below, whose positions are specified according to EU numbering (each number represents the position of an amino acid residue in the EU numbering; and the one-letter amino acid symbol before the number represents the amino acid residue before substitution, while the one-letter amino acid symbol after the number represents the amino acid residue after the substitution) in the amino acids forming the Fc domain of IgG1 antibody:
(a) L234F, L235E, P331S;
(b) C226S, C229S, P238S;
(c) C226S, C229S; or
(d) C226S, C229S, E233P, L234V, L235A;
as well as those having an Fc domain which has a deletion of the amino acid sequence at positions 231 to 238.

Furthermore, the preferred antigen-binding molecules or antibodies also include those comprising an Fc domain that has any one of the substitutions shown below, whose positions are specified according to EU numbering in the amino acids forming the Fc domain of an IgG2 antibody:
(e) H268Q, V309L, A330S, and P331S;
(f) V234A;
(g) G237A;
(h) V234A and G237A;
(i) A235E and G237A; or
(j) V234A, A235E, and G237A.

Each number represents the position of an amino acid residue in EU numbering; and the one-letter amino acid symbol before the number represents the amino acid residue before substitution, while the one-letter amino acid symbol after the number represents the amino acid residue after the substitution.

Furthermore, the preferred antigen-binding molecules or antibodies also include those comprising an Fc domain that has any one of the substitutions shown below, whose positions are specified according to EU numbering in the amino acids forming the Fc domain of an IgG3 antibody:
(k) F241A;
(l) D265A; or
(m) V264A.

Each number represents the position of an amino acid residue in EU numbering; and the one-letter amino acid symbol before the number represents the amino acid residue before substitution, while the one-letter amino acid symbol after the number represents the amino acid residue after the substitution.

Furthermore, the preferred antigen-binding molecules or antibodies also include those comprising an Fc domain that has any one of the substitutions shown below, whose positions are specified according to EU numbering in the amino acids forming the Fc domain of an IgG4 antibody:
(n) L235A, G237A, and E318A;
(o) L235E; or
(p) F234A and L235A.

Each number represents the position of an amino acid residue in EU numbering; and the one-letter amino acid symbol before the number represents the amino acid residue before substitution, while the one-letter amino acid symbol after the number represents the amino acid residue after the substitution.

The other preferred antigen-binding molecules or antibodies include, for example, those comprising an Fc domain in which any amino acid at position 233, 234, 235, 236, 237, 327, 330, or 331 (EU numbering) in the amino acids forming the Fc domain of an IgG1 antibody is substituted with an amino acid of the corresponding position in EU numbering in the corresponding IgG2 or IgG4.

The preferred antigen-binding molecules or antibodies also include, for example, those comprising an Fc domain in which any one or more of the amino acids at positions 234, 235, and 297 (EU numbering) in the amino acids forming the Fc domain of an IgG1 antibody is substituted with other amino acids. The type of amino acid after substitution is not particularly limited; however, the antigen-binding molecules or antibodies comprising an Fc domain in which any one or more of the amino acids at positions 234, 235, and 297 are substituted with alanine are particularly preferred.

The preferred antigen-binding molecules or antibodies also include, for example, those comprising an Fc domain in which an amino acid at position 265 (EU numbering) in the amino acids forming the Fc domain of an IgG1 antibody is substituted with another amino acid. The type of amino acid after substitution is not particularly limited; however, antigen-binding molecules or antibodies comprising an Fc domain in which an amino acid at position 265 is substituted with alanine are particularly preferred.

### Antigen-Binding Domains Bind to HER2 S310F

The phrase “an antigen-binding domain binds to HER2 S310F” or “an anti-HER2 S310F antigen-binding domain” as used herein refers to an antigen-binding domain that specifically binds to the above-mentioned HER2 S310F protein, or the whole or a portion of a partial peptide of the HER2 S310F protein.

In certain embodiments, the antigen-binding domain binds to HER2 S310F is a domain comprising antibody variable region (antibody light-chain and heavy-chain variable regions (VL and VH)). Suitable examples of such domains comprising antibody light-chain and heavy-chain variable regions include “single chain Fv (scFv)”, “single chain antibody”, “Fv”, “single chain Fv2 (scFv2)”, “Fab”, “F(ab′)2”, etc. In specific embodiments, the antigen-binding domain binds to HER2 S310F is a domain comprising an antibody variable fragment. Domains comprising an antibody variable fragment may be provided from variable domains of one or a plurality of antibodies.

In certain embodiments, the antigen-binding domain binds to HER2 S310F comprises the heavy-chain variable region and light-chain variable region of an anti-HER2 S310F antibody. In certain embodiments, the antigen-binding domain binds to HER2 S310F is a domain comprising a Fab structure.

Preferably, the anti-HER2 S310F antibody comprises an H chain comprising the amino acid sequence (H-chain variable region) and an L chain comprising the amino acid sequence (L-chain variable region) as described in the Table 1, respectively.

In some embodiments, the anti-HER2 S310F antigen-binding domain binds specifically to the extracellular domain of HER2 S310F (amino acids 23-652 of SEQ ID NO: 166). In some embodiments, the anti-HER2 S310F antigen-binding domain binds specifically to an epitope within extracellular domain of HER2 S310F which comprises an amino acid residue Phenylalanine correspond to S310F position. In some embodiments, the anti-HER2 S310F antigen-binding domain does not bind to human wild type HER2, preferably human wild type HER2 expressed on the surface of human cells. In some embodiments, the anti-HER2 S310F antigen-binding domain does not bind, or does not substantially bind to a wild type HER2 isoforms from human, including as disclosed in RefSeq accession numbers NP_001005862.1(receptor tyrosine-protein kinase erbB-2 isoform b; SEQ ID NO: 162), NP_001276865.1 (receptor tyrosine-protein kinase erbB-2 isoform c; SEQ ID NO: 163), NP_001276866.1 (receptor tyrosine-protein kinase erbB-2 isoform d; SEQ ID NO: 164), NP_001276867.1 (receptor tyrosine-protein kinase erbB-2 isoform e; SEQ ID NO: 165), NP_004439.2 (receptor tyrosine-protein kinase erbB-2 isoform a; SEQ ID NO: 166), preferably expressed on the surface of human cells without HER2 S310F mutation. In some embodiments, the anti-HER2 S310F antigen-binding domain binds to the HER2 S310F protein expressed on the surface of eukaryotic cells. In some embodiments, the anti-HER2 S310F antigen-binding domain binds to the HER2 S310F protein expressed on the surface of cancer cells.

In specific embodiments, the antigen-binding domain binds to HER2 S310F comprises any one of the antibody variable fragments shown in Table 1 below.

TABLE 1
Sequences of HVRs or VH, VL in an antigen-binding domain binds to
HER2 S310F

| Antibody | Heavy chain | HVR- | HVR- | HVR- | Light chain | HVR- | HVR- | HVR- |
|---|---|---|---|---|---|---|---|---|
| Name | variable region | H1 | H2 | H3 | variable region | L1 | L2 | L3 |
| MHR | QSVEESGGRLVTP | TYDM | YIYAG | GGAG | ARVLTQTPSPV | QASQ | YAAT | QGYF |
| 0007 | GTPLTLTCTVSGF | S | SIGTY | ASFDF | SAAVGGTVTIT | SVYS | LAS | SGGV |
| | SLSTYDMSWVRQ | | YASW | | CQASQSVYSN | NNLL | | YT |
| | APGKGLEW1GYI | | VNG | | NLLSWYQQKP | S | | |
| | YAGSIGTYYASW | | | | GQPPKLLIYYA | | | |
| | VNGRFTISKTSTT | | | | ATLASGVPSRF | | | |
| | MDLRMTSLTAAD | | | | KGSGSGTQFTL | | | |
| | TATYFCARGGAG | | | | TISDIQCDDAA | | | |
| | ASFDFWGPGTLV | | | | TYYCQGYFSG | | | |
| | TVSS | | | | GVYTFGGGTK | | | |
| | | | | | VEIK | | | |
| MHR | QSLEESGGRLVTP | SYAMI | MVYIH | YNGG | DIVMTQTPSSV | QASQ | YAST | QNN |
| 0009 | GTPLTLTCKASGF | | SGGTY | GSGT | SEPVGGTVTIK | SIYSY | LAS | YGSD |
| | SLSSYAMIWVRQ | | YASW | DL | CQASQSIYSYL | LS | | SNIY |
| | APGKGLESIGMV | | AKG | | SWYQQKPGQR | | | GIA |
| | YIHSGGTYYASW | | | | PKLLIYYASTL | | | |
| | AKGRFTISKTSTT | | | | ASGVPSRFKGS | | | |
| | VDLKITSPTTEDT | | | | GSGTQFTLTIS | | | |
| | ATYFCARYNGGG | | | | DLECADAATY | | | |
| | SGTDLWGPGTLV | | | | YCQNNYGSDS | | | |
| | TVSS | | | | NIYGIAFGGGT | | | |
| | | | | | KVEIK | | | |
| MHR | QSVEESGGRLVTP | RYAM | IIVSSS | IRGYD | DVVMTQTPAS | QASQ | DASD | QSYY |
| 0010 | GTPLTLTCTVSGF | S | GSTYY | NIGY | VEAAVGGTVT | SISSY | LAS | YSSS |
| | SLSRYAMSWVRQ | | ATWA | GYPW | IKCQASQSISS | LA | | SSYG |
| | APGKGLEWIGIIV | | TG | FYYF | YLAWYQQKP | | | FA |
| | SSSGSTYYATWA | | | NL | GQPPKLLIYDA | | | |
| | TGRFTISKTSTTV | | | | SDLASGVPSRF | | | |
| | DLKITSPTTEDTA | | | | KGSGSGTQFTL | | | |
| | TYFCARIRGYDNI | | | | TISDLECADAA | | | |
| | GYGYPWFYYFNL | | | | TYYCQSYYYS | | | |
| | WGPGTLVTVSS | | | | SSSSYGFAFGG | | | |
| | | | | | GTKVEIK | | | |
| MHR | QSVEESGGRLVTP | SNAIS | IISSSG | GVDI | EVVMTQTPAS | QASQ | YAST | QSNY |
| 0012 | GTPLTLTCTVSGF | | STYYA | YGHA | VSEPVGGTVTI | SISSY | LAS | GSSS |
| | SLSSNAISWVRQA | | SWAK | YATP | KCQASQSISSY | LA | | SSYE |
| | PGKGLEYIGIISSS | | G | YGMD | LAWYQQKPG | | | DT |
| | GSTYYASWAKGR | | | L | QPPKRLIYYAS | | | |
| | FTISKTSTTVDLK1 | | | | TLASGVPSRFK | | | |
| | TSPTTEDTATYFC | | | | GSGSGTEFTLT | | | |
| | ARGVDIYGHAYA | | | | ISDLECDDAAT | | | |
| | TPYGMDLWGPGT | | | | YYCQSNYGSS | | | |
| | LVTVSS | | | | SSSYEDTFGGG | | | |
| | | | | | TKVEIK | | | |
| MHR | QSVEESGGRLVTP | NYDM | YISSSG | DGSSS | AYDMTQTPAS | QASQ | TAFY | QQG |
| 0014 | GTPLTLTCTVSGF | T | STSYA | DYDR | VEVAVGGTVT | NIYSF | LAS | YSRS |
| | SLSNYDMTWVR | | SWAK | LDL | IKCQASQNIYS | LA | | NVD |
| | QTPGKGLEWIGYI | | G | | FLAWYQQKPG | | | NT |
| | SSSGSTSYASWA | | | | QPPKLLIYTAF | | | |
| | KGRFTISKTSTTV | | | | YLASGVPSRFK | | | |
| | DLKMTSLTTEDT | | | | GSGSGTEYTLT | | | |
| | ATYFCARDGSSS | | | | ISGVECADAAT | | | |
| | DYDRLDLWGQG | | | | YYCQQGYSRS | | | |
| | TLVTVSS | | | | NVDNTEGGGT | | | |
| | | | | | KVEIK | | | |
| MHR | QSVEESGGRLVTP | SNAM | IISSSG | GVDI | EVVMTQTPAS | QASQ | YSST | QSNY |
| 0015 | GTPLTLTCTVSGF | G | STYYA | YGHA | VSEPVGGTVTI | SISSY | LAS | GSSS |
| | SLSSNAMGWVRQ | | SWAK | YATP | KCQASQSISSY | LS | | SSYE |
| | APGKGLEYIGIISS | | G | YGMD | LSWYQQKPGQ | | | DT |
| | SGSTYYASWAKG | | | L | PPKRLIYYSST | | | |
| | RFTISKTSTTVDL | | | | LASGVPSRFKG | | | |
| | KITSPTTEDTATY | | | | SGSGTEFTLTIS | | | |
| | FCARGVDIYGHA | | | | DLECDDAATY | | | |
| | YATPYGMDLWG | | | | YCQSNYGSSSS | | | |
| | PGTLVTVSS | | | | SYEDTEGGGT | | | |
| | | | | | KVEIK | | | |
| MHR | QSVEESGGRLVTP | SNAIS | IISSSG | GVDI | EVVMTQTPAS | QASQ | YSST | QSNY |
| 0016 | GTPLTLTCTVSGF | | STYYA | YGHA | VSEPVGGTVTI | SISSY | LAS | GSSS |
| | SLSSNAISWVRQA | | NWAK | YATP | KCQASQSISSY | LS | | SSYE |
| | PGKGLEYIGIISSS | | G | YGMD | LSWYQQKPGQ | | | DT |
| | GSTYYANWAKG | | | L | PPKLLIYYSST | | | |
| | RFTISKTSSTTVD | | | | LASGVPSRFKG | | | |
| | LKMTSATTEDTA | | | | SGSGTEFTLTIS | | | |
| | TYFCARGVDIYG | | | | DLECADAATY | | | |
| | HAYATPYGMDL | | | | YCQSNYGSSSS | | | |
| | WGPGTLVTVSS | | | | SYEDTFGGGT | | | |
| | | | | | KVEIK | | | |
| MHR | QSLEESGGRLVTP | SYAMI | MVYIH | YNGG | DIVMTQTPSSV | QASQ | YAST | QNN |
| 0017 | GTPLTLTCKASGF | | SGGTY | GSGT | SEPVGGTVTIK | SIYSY | LAS | YGSD |
| | SLSSYAMIWVRQ | | YASW | DL | CQASQSIYSYL | LS | | SNIY |
| | APGKGLESIGMV | | AKG | | SWYQQKPGQR | | | GIA |
| | YIHSGGTYYASW | | | | PKLLIYYASTL | | | |
| | AKGRFTISKTSTT | | | | ASGVPSRFKGS | | | |
| | VDLKITSPTTEDT | | | | GSGTQFTLTIS | | | |
| | ATYFCAKYNGGG | | | | DLECADAATY | | | |
| | SGTDLWGPGTLV | | | | YCQNNYGSDS | | | |
| | TVSS | | | | NIYGIAFGGGT | | | |
| | | | | | KVEIK | | | |
| MHR | QSVEESGGRLVTP | SNAM | IISSSG | GVDI | EVVMTQTPAS | QASQ | YSST | QSNY |
| 0018 | GTPLTLTCTVSGF | G | STYYA | YGHA | VSEPVGGTVTI | SISSY | LAS | GSSS |
| | SLSSNAMGWVRQ | | SWAK | YATP | KCQASQSISSY | LS | | SSYE |
| | APGKGLEYIGIISS | | G | YGMD | LSWYQQKPGQ | | | DT |
| | SGSTYYASWAKG | | | L | PPKRLIYYSST | | | |
| | RFTISKTSTTVDL | | | | LASGVPSRFKG | | | |
| | KITSPTTEDTATY | | | | SGSGTEFTLTIS | | | |
| | FCARGVDIYGHA | | | | DLECDDAATY | | | |
| | YATPYGMDLWG | | | | YCQSNYGSSSS | | | |
| | PGTLVTVSS | | | | SYEDTFGGGT | | | |
| | | | | | KVEIK | | | |
| MHR | QSVEESGGRLVA | SDAM | IISSSG | GVDI | EVVMTQTPAS | QASQ | YAST | QSNY |
| 0019 | PGTPLTLTCTVSG | A | STYYA | YGHA | VSEPVGGTVTI | SISSY | LAS | GSSS |
| | IDLSSDAMAWVR | | SWAK | YATP | KCQASQSISSY | LS | | SSYE |
| | QAPGKGLEYIGIIS | | G | YGMD | LSWYQQKPGQ | | | DT |
| | SSGSTYYASWAK | | | L | PPKLLIYYSST | | | |
| | GRFTISKTSTTVD | | | | LASGVPSRFKG | | | |
| | LKITSPTTEDTAT | | | | SGSGTEFTLTIS | | | |
| | YFCARGVDIYGH | | | | DLECADAATY | | | |
| | AYATPYGMDLW | | | | YCQSNYGSSSS | | | |
| | GPGTLVTVSS | | | | SYEDTFGGGT | | | |
| | | | | | KVEIK | | | |
| MHR | QSVEESGGRLVTP | SYAM | IISSSG | GVDI | EVVMTQTPAS | QASQ | YAST | QSNY |
| 0060 | GTPLTLTCTVSGF | G | STYYA | YGHP | VSEPVGGTVTI | SISSY | LAS | GSSS |
| | SLSSYAMGWVRQ | | NWAK | YATP | KCQASQSISSY | LA | | SSYE |
| | APGKGLEYIGIISS | | G | YGMD | LSWYQQKPGQ | | | DT |
| | SGSTYYANWAK | | | L | PPKLLIYYSST | | | |
| | GRFTISKTSTTVD | | | | LASGVPSRFKG | | | |
| | LKITSPTTEDTAT | | | | SGSGTEFTLTIS | | | |
| | YFCARGVDIYGH | | | | DLECADAATY | | | |
| | PYATPYGMDLW | | | | YCQSNYGSSSS | | | |
| | GPGTLVTVSS | | | | SYEDTFGGGT | | | |
| | | | | | KVEIK | | | |
| MHR | QSVEESGGRLVTP | SNAIS | IISSNGI | GVDI | EVVMTQTPAS | QASQ | YAST | QSNY |
| 0082 | GTPLTLTCTVSGF | | TYYAS | YGHA | VSEPVGGTVTI | SISSY | LAS | GSSS |
| | SLSSNAISWVRQA | | WAKG | YATP | KCQASQSISSY | LS | | SSYE |
| | PGKGLQYIGIISSN | | | YGMD | LSWYQQKPGQ | | | DT |
| | GITYYASWAKGR | | | L | PPKRLIYYAST | | | |
| | FTISKTSTTVDLKI | | | | LASGVPSRFKG | | | |
| | TSPTTEDTATYFC | | | | SGSGTEFTLTIS | | | |
| | ARGVDIYGHAYA | | | | DLECDDAATY | | | |
| | TPYGMDLWGPGT | | | | YCQSNYGSSSS | | | |
| | LVTVSS | | | | SYEDTFGGGT | | | |
| | | | | | KVEIK | | | |

In specific embodiments, the antigen-binding domain binds to HTER2 S310F is a domain that comprises an antibody variable fragment that competes for binding to human HER2 S310F with any one of the antibody variable fragments shown in Table 2. In specific embodiments, the antigen-binding domain binds to HER2 S310F is a domain that comprises an antibody variable fragment that binds to the same epitope within human HER2 S310F as any one of the antibody variable fragments shown in Table 2.

Alternatively, the antigen-binding domain binds to HER2 S310F comprises an antibody variable fragment that competes for binding to human HER2 S310F with any one of the above-mentioned antibody variable fragments. Alternatively, the antigen-binding domain binds to HER2 S310F comprises an antibody variable fragment that binds to the same epitope to which any one of the above-mentioned antibody variable fragments binds on human HER2 S310F.

Antigen-binding domains bind to T cell receptor complex The phrase “an antigen-binding domain binds to T cell receptor complex” or “an anti-T cell receptor complex antigen-binding domain” as used herein refers to an antigen-binding domain that specifically binds to the whole or a portion of a partial peptide of a T cell receptor complex. The T cell receptor complex may be a T cell receptor itself, or an adaptor molecule constituting a T cell receptor complex along with a T cell receptor. CD3 is suitable as an adaptor molecule.

In certain embodiments, the antigen-binding domain binds to T cell receptor complex-binding activity is a domain comprising antibody variable region (antibody light-chain and heavy-chain variable regions (VL and VH)). Suitable examples of such domains comprising antibody light-chain and heavy-chain variable regions include “single chain Fv (scFv)”, “single chain antibody”, “Fv”, “single chain Fv2 (scFv2)”, “Fab”, “F(ab′)2”, etc. In specific embodiments, the antigen-binding domain binds to T cell receptor complex is a domain comprising an antibody variable fragment. Domains comprising an antibody variable fragment may be provided from variable domains of one or a plurality of antibodies.

In certain embodiments, the antigen-binding domain binds to T cell receptor complex-comprises the heavy-chain variable region and light-chain variable region of an anti-T cell receptor complex antibody. In certain embodiments, the antigen-binding domain binds to T cell receptor complex is a domain comprising a Fab structure.

### Antigen-Binding Domains Bind to T Cell Receptor

The phrase “an antigen-binding domain binds to T cell receptor” or “an anti-T cell receptor antigen-binding domain” as used herein refers to an antigen-binding domain that specifically binds to the whole or a portion of a partial peptide of a T cell receptor. The portion of a T cell receptor to which the antigen-binding domain binds may be a variable region of the T cell receptor or a constant region of the T cell receptor; however, an epitope present in the constant region is preferred. Examples of the constant region sequence include the T cell receptor alpha chain of RefSeq Accession No. CAA26636.1 (SEQ ID NO: 140), the T cell receptor beta chain of RefSeq Accession No. C25777 (SEQ ID NO: 141), the T cell receptor gamma 1 chain of RefSeq Accession No. A26659 (SEQ ID NO: 142), the T cell receptor gamma 2 chain of RefSeq Accession No. AAB63312.1 (SEQ ID NO: 143), and the T cell receptor delta chain of RefSeq Accession No. AAA61033.1 (SEQ ID NO: 144).

In certain embodiments, the antigen-binding domain binds to T cell receptor is a domain comprising antibody variable region (antibody light-chain and heavy-chain variable regions (VL and VH)). Suitable examples of such domains comprising antibody light-chain and heavy-chain variable regions include “single chain Fv (scFv)”, “single chain antibody”, “Fv”, “single chain Fv2 (scFv2)”, “Fab”, “F(ab′)2”, etc. In specific embodiments, the antigen-binding domain binds to T cell receptor is a domain comprising an antibody variable fragment. Domains comprising an antibody variable fragment may be provided from variable domains of one or a plurality of antibodies.

In certain embodiments, the antigen-binding domain binds to T cell receptor comprises the heavy-chain variable region and light-chain variable region of an anti-T cell receptor antibody. In certain embodiments, the antigen-binding binds to T cell receptor is a domain comprising a Fab structure.

### Antigen-Binding Domains Bind to CD3

The phrase “an antigen-binding domain binds to CD3” or “an anti-CD3 antigen-binding domain” as used herein refers to an antigen-binding domain that specifically binds to the whole or a portion of a partial peptide of CD3. The antigen-binding domain binds to CD3 may be any epitope-binding domain as long as the epitope exists in the gamma-chain, delta-chain, or epsilon-chain sequence that constitutes human CD3. Regarding the structure of the gamma chain, delta chain, or epsilon chain constituting CD3, their polynucleotide sequences are disclosed in RefSeq Accession NOs. NM_000073.2, NM_000732.4 and NM_000733.3, and their polypeptide sequences are shown in SEQ ID NOs: 145 (NP_000064.1), 146 (NP_000723.1), and 147 (NP_000724.1), wherein the RefSeq accession numbers are shown in parentheses.

In certain embodiments, the antigen-binding domain binds to CD3 is a domain comprising antibody light-chain and heavy-chain variable regions (VL and VH). Suitable examples of such domains comprising antibody light-chain and heavy-chain variable regions include “single chain Fv (scFv)”, “single chain antibody”, “Fv”, “single chain Fv 2 (scFv2)”, “Fab”, “F(ab′)2”, etc. In specific embodiments, the antigen-binding domain binds to CD3 is a domain comprising an antibody variable fragment. Domains comprising an antibody variable fragment may be provided from variable domains of one or a plurality of antibodies.

In certain embodiments, the antigen-binding domain binds to CD3 comprises the heavy-chain variable region and light-chain variable region of an anti-CD3 antibody. In certain embodiments, the antigen-binding domain binds to CD3 is a domain comprising a Fab structure.

The antigen-binding domains bind to CD3-binding activity of the present invention may bind to any epitope, as long as the epitope is located within the gamma chain, delta chain, or epsilon chain sequence forming human CD3. In the present invention, preferred antigen-binding domains bind to CD3 include those comprising a CD3 antibody light-chain variable region (VL) and a CD3 antibody heavy-chain variable region (VH), which bind to an epitope in the extracellular domain of the epsilon chain of a human CD3 complex. Such preferred antigen-binding domains bind to CD3 include those comprising a CD3 antibody light-chain variable region (VL) and a CD3 antibody heavy-chain variable region (VH) of the OKT3 antibody (Proc. Natl. Acad. Sci. USA (1980) 77, 4914-4917) or various known CD3 antibodies such as an antibody with the light-chain variable region (VL) of NCBI Accession No. AAB24132 and the heavy-chain variable region (VH) of NCBI Accession No. AAB24133 (Int. J. Cancer Suppl. 7, 45-50 (1992)). Several antibodies that bind to different epitopes of human CD3 epsilon are known in the art, e.g. the antibody OKT3 (see e.g. Kung, P. et al, Science 206 (1979) 347-349; Salmeron, A. et al, J Immunol 147 (1991) 3047-3052; U.S. Pat. No. 9,226,962B2), the antibody UCHT1 (see e.g. Callard, R. E. et al, Clin Exp Immunol 43 (1981) 497-505; Arnett et al. PNAS 2004) or the antibody SP34 (human cynomolgus CD3 cross-reactive; see e.g. Pessano, S. et al, EMBO J 4 (1985) 337-344, Conrad M. L., et. al, Cytometry A 71 (2007) 925-933). WO2015181098 also discloses human cynomolgus cross-reactive antibody specifically binds to human and cynomolgus T cells, activates human T cells and does not bind to the same epitope as the antibody OKT3, the antibody UCHT1 and/or antibody the SP34. Furthermore, such appropriate antigen-binding domains bind to CD3 include those derived from a CD3 antibody with desired characteristics, which are obtained by immunizing a desired animal with the gamma chain, delta chain, or epsilon chain forming human CD3 by the above-described methods. Appropriate anti-CD3 antibodies from which an antigen-binding domain binds to CD3 is derived include human antibodies and antibodies appropriately humanized as described above.

### Multispecific Antigen-Binding Molecules

“Multispecific antigen-binding molecules” refers to antigen-binding molecules that bind specifically to more than one antigen. In a favorable embodiment, multispecific antigen-binding molecules of the present invention comprise two or more antigen-binding domains, and different antigen-binding domains bind specifically to different antigens.

The multispecific antigen-binding molecule of the present invention comprises a first antigen-binding domain binds to HER2 S310F, and a second antigen-binding domain binds to T cell receptor complex. The combinations of an antigen-binding domain binds to HER2 S310F selected from those described in “Antigen-binding domains bind to HER2 S310F” above and an antigen-binding domain binds to T cell receptor complex selected from those described in “Antigen-binding domains bind to T-cell receptor complex” to “Antigen-binding domains binds to CD3” above can be used.

For example, the first antigen-binding domain is a domain comprising antibody heavy-chain and light-chain variable regions, and/or the second antigen-binding domain is a domain comprising antibody heavy-chain and light-chain variable regions. Alternatively, the first antigen-binding domain is a domain comprising an antibody variable fragment, and/or the second antigen-binding domain is a domain comprising an antibody variable fragment. Alternatively, the first antigen-binding domain is a domain comprising a Fab structure, and/or the second antigen-binding domain is a domain comprising a Fab structure.

In certain embodiments, the present invention provides a multispecific antigen-binding molecule comprising a first antigen-binding domain that comprises an antibody variable fragment that binds to HER2 S310F, and a second antigen-binding domain that comprises an antibody variable fragment that binds to T cell receptor complex. In certain embodiments, the present invention provides bispecific antigen-binding molecules that comprise a first antigen-binding domain binds to HER2 S310F, a second antigen-binding domain binds to T cell receptor complex, and a third domain comprising an Fc region that has a reduced Fc gamma receptor-binding activity. The Fc region may have a reduced Fc gamma receptor-binding activity compared with the Fc domain of an IgG1, IgG2, IgG3, or IgG4 antibody. In an embodiment, the Fc region is an Fc region with an amino acid mutation at any of the Fc region-constituting amino acids of SEQ ID NOs: 148 to 151 (IgG1 to IgG4).

In certain embodiments, the present invention provides bispecific antibodies that comprise a first antibody variable fragment that binds to human HER2 S310F, and a second antibody variable fragment that binds to CD3. In certain embodiments, the present invention provides bispecific antibodies that comprise a first antibody variable fragment binds to human HER2 S310F, a second antibody variable fragment binds to CD3, and an Fc region that has a reduced Fc gamma receptor-binding activity. In certain embodiments, the present invention provides bispecific antibodies that comprise a first antibody variable fragment binds to human HER2 S310F, a second antibody variable fragment that binds to CD3 eplison chain, and an Fc region that has a reduced Fc gamma receptor-binding activity compared with naturally occurring IgG Fc regions.

Examples of a preferred embodiment of the “multispecific antigen-binding molecule” of the present invention include multispecific antibodies. When an Fc region with reduced Fc gamma receptor-binding activity is used as the multispecific antibody Fc region, an Fc region derived from the multispecific antibody may be used appropriately. Bispecific antibodies are particularly preferred as the multispecific antibodies of the present invention. In this case, a bispecific antibody is an antibody having two different specificities. IgG-type bispecific antibodies can be secreted from a hybrid hybridoma (quadroma) produced by fusing two types of hybridomas that produce IgG antibodies (Milstein et al., Nature (1983) 305, 537-540).

Furthermore, IgG-type bispecific antibodies are secreted by introducing the genes of L chains and H chains constituting the two types of IgGs of interest, i.e., a total of four genes, into cells, and co-expressing them. However, the number of combinations of H and L chains of IgG that can be produced by these methods is theoretically ten combinations. Accordingly, it is difficult to purify an IgG comprising the desired combination of H and L chains from ten types of IgGs. Furthermore, theoretically, the amount of secretion of the IgG having the desired combination will decrease remarkably, and therefore large-scale culturing will be necessary, and production costs will increase further.

Therefore, techniques for promoting the association among H chains and between L and H chains having the desired combinations can be applied to the multispecific antigen-binding molecules of the present invention.

For example, techniques for suppressing undesired H-chain association by introducing electrostatic repulsion at the interface of the second constant region or the third constant region of the antibody H chain (CH2 or CH3) can be applied to multispecific antibody association (WO2006/106905).

In the technique of suppressing unintended H-chain association by introducing electrostatic repulsion at the interface of CH2 or CH3, examples of amino acid residues in contact at the interface of the other constant region of the H chain include regions corresponding to the residues at EU numbering positions 356, 439, 357, 370, 399, and 409 in the CH3 region.

More specifically, examples include an antibody comprising two types of H-chain CH3 regions, in which one to three pairs of amino acid residues in the first H-chain CH3 region, selected from the pairs of amino acid residues indicated in (1) to (3) below, carry the same type of charge: (1) amino acid residues comprised in the H chain CH3 region at EU numbering positions 356 and 439; (2) amino acid residues comprised in the H-chain CH3 region at EU numbering positions 357 and 370; and (3) amino acid residues comprised in the H-chain CH3 region at EU numbering positions 399 and 409.

Furthermore, the antibody may be an antibody in which pairs of the amino acid residues in the second H-chain CH3 region which is different from the first H-chain CH3 region mentioned above, are selected from the aforementioned pairs of amino acid residues of (1) to (3), wherein the one to three pairs of amino acid residues that correspond to the aforementioned pairs of amino acid residues of (1) to (3) carrying the same type of charges in the first H-chain CH3 region mentioned above carry opposite charges from the corresponding amino acid residues in the first H-chain CH3 region mentioned above.

Each of the amino acid residues indicated in (1) to (3) above come close to each other during association. Those skilled in the art can find out positions that correspond to the above-mentioned amino acid residues of (1) to (3) in a desired H-chain CH3 region or H-chain constant region by homology modeling and such using commercially available software, and amino acid residues of these positions can be appropriately subjected to modification.

In the antibodies mentioned above, “charged amino acid residues” are preferably selected, for example, from amino acid residues included in either one of the following groups:
(a) glutamic acid (E) and aspartic acid (D); and
(b) lysine (K), arginine (R), and histidine (H).

In the above-mentioned antibodies, the phrase “carrying the same charge” means, for example, that all of the two or more amino acid residues are selected from the amino acid residues included in either one of groups (a) and (b) mentioned above. The phrase “carrying opposite charges” means, for example, that when at least one of the amino acid residues among two or more amino acid residues is selected from the amino acid residues included in either one of groups (a) and (b) mentioned above, the remaining amino acid residues are selected from the amino acid residues included in the other group.

In a preferred embodiment, the antibodies mentioned above may have their first H-chain CH3 region and second H-chain CH3 region crosslinked by disulfide bonds.

In the present invention, amino acid residues subjected to modification are not limited to the above-mentioned amino acid residues of the antibody variable regions or the antibody constant regions. Those skilled in the art can identify the amino acid residues that form an interface in mutant polypeptides or heteromultimers by homology modeling and such using commercially available software; and amino acid residues of these positions can then be subjected to modification so as to regulate the association.

Other known techniques can also be used for the association of multispecific antibodies of the present invention. Fc region-containing polypeptides comprising different amino acids can be efficiently associated with each other by substituting an amino acid side chain present in one of the H-chain Fc regions of the antibody with a larger side chain (knob), and substituting an amino acid side chain present in the corresponding Fc region of the other H chain with a smaller side chain (hole) to allow placement of the knob within the hole (WO1996/027011; Ridgway J B et al., Protein Engineering (1996) 9, 617-621; Merchant A. M. et al. Nature Biotechnology (1998) 16, 677-681; and US20130336973).

In addition, other known techniques can also be used for formation of multispecific antibodies of the present invention. Association of polypeptides having different sequences can be induced efficiently by complementary association of CH3 using a strand-exchange engineered domain CH3 produced by changing part of one of the H-chain CH3s of an antibody to a corresponding IgA-derived sequence and introducing a corresponding IgA-derived sequence into the complementary portion of the other H-chain CH3 (Protein Engineering Design & Selection, 23; 195-202, 2010). This known technique can also be used to efficiently form multispecific antibodies of interest.

In addition, technologies for antibody production using association of antibody CH1 and CL and association of VH and VL as described in WO 2011/028952, WO2014/018572, and Nat Biotechnol. 2014 February; 32(2):191-8; technologies for producing bispecific antibodies using separately prepared monoclonal antibodies in combination (Fab Arm Exchange) as described in WO2008/119353, WO2011/131746, WO2015/046467 and WO2016159213; technologies for regulating association between antibody heavy-chain CH3s as described in WO2012/058768 and WO2013/063702; technologies for producing bispecific antibodies composed of two types of light chains and one type of heavy chain as described in WO2012/023053; technologies for producing bispecific antibodies using two bacterial cell strains that individually express one of the chains of an antibody comprising a single H chain and a single L chain as described by Christoph et al. (Nature Biotechnology Vol. 31, p 753-758 (2013)); and such may be used for the formation of multispecific antibodies.

Alternatively, even when a multispecific antibody of interest cannot be formed efficiently, a multispecific antibody of the present invention can be obtained by separating and purifying the multispecific antibody of interest from the produced antibodies. For example, a method for enabling purification of two types of homomeric forms and the heteromeric antibody of interest by ion-exchange chromatography by imparting a difference in isoelectric points by introducing amino acid substitutions into the variable regions of the two types of H chains has been reported (WO2007114325). To date, as a method for purifying heteromeric antibodies, methods using Protein A to purify a heterodimeric antibody comprising a mouse IgG2a H chain that binds to Protein A and a rat IgG2b H chain that does not bind to Protein A have been reported (WO98050431 and WO95033844). Furthermore, a heterodimeric antibody can be purified efficiently on its own by using H chains comprising substitution of amino acid residues at EU numbering positions 435 and 436, which is the IgG-Protein A binding site, with Tyr, His, or such which are amino acids that yield a different Protein A affinity, or using H chains with a different protein A affinity, to change the interaction of each of the H chains with Protein A, and then using a Protein A column.

Furthermore, an Fc region whose Fc region C-terminal heterogeneity has been improved can be appropriately used as an Fc region of the present invention. More specifically, the present invention provides Fc regions produced by deleting glycine at position 446 and lysine at position 447 as specified by EU numbering from the amino acid sequences of two polypeptides constituting an Fc region derived from IgG1, IgG2, IgG3, or IgG4.

A plurality, such as two or more, of these technologies can be used in combination. Furthermore, these technologies can be appropriately and separately applied to the two H chains to be associated. Furthermore, these techniques can be used in combination with the above-mentioned Fc region which has reduced binding activity to an Fc gamma receptor. Furthermore, an antigen-binding molecule of the present invention may be a molecule produced separately so that it has the same amino acid sequence, based on the antigen-binding molecule subjected to the above-described modifications.

Preferably, the antigen-binding molecule of the present invention may comprise a first antigen-binding domain binds to HER2 S310F, and a second antigen-binding domain binds to T cell receptor complex. In an embodiment, the second antigen-binding domain binds to T cell receptor. In another embodiment, the second antigen-binding domain binds to CD3 epsilon chain. In an embodiment, the first antigen-binding domain binds to human HER2 S310F. In a further embodiment, the first antigen-binding domain binds to HER2 S310F on the surface of a eukaryotic cell. In an embodiment, the first antigen-binding domain binds to human HER2 S310F on the surface of a eukaryotic cell including cancer cell.

The phrase “anti-HER2 S310F arm” in this specification refers to the antibody heavy chain and antibody light chain which binds to HER2 S310F in a bispecific antibody. The phrase “anti-CD3 arm” in this specification refers to the antibody heavy chain and antibody light chain which binds to CD3 in a bispecific antibody.

Preferably, the antigen-binding molecule of the present invention may have cellular cytotoxicity (also referred to as “cytotoxicity”). In an embodiment, the cellular cytotoxicity is T cell-dependent cellular cytotoxicity (TDCC). In another embodiment, the cytotoxicity is a cellular cytotoxicity towards cells expressing HER2 S310F on their surfaces. The HER2 S310F-expressing cells may be cancer cells.

In a preferred aspect, an antibody (or antigen-binding molecule) of the present invention has cytotoxicity (or cellular cytotoxicity), or preferably T cell-dependent cellular cytotoxicity (TDCC) against HER2 S310F-expressing cells such as cancer cells. HER2 S310F may be expressed on the surface of such cells. The (cellular) cytotoxicity or TDCC of an antibody (or antigen-binding molecule) of the present invention can be evaluated by any suitable method known in the art. For example, TDCC can be measured by lactate dehydrogenase (LDH) release assay as described in Example 5.2. In this assay, target cells (e.g. HER2 S310F-expressing cells) are incubated with T cells (e.g. PBMCs) or expanded T cells in the presence of a test antibody, and the activity of LDH that has been released from target cells killed by T cells is measured using a suitable reagent. Typically, the cytotoxic activity is calculated as a percentage of the LDH activity resulting from the incubation with the antibody relative to the LDH activity resulting from 100% killing of target cells (e.g. lysed by treatment with Triton-X). If the cytotoxic activity calculated as mentioned above is higher, the test antibody is determined to have higher TDCC. Additionally or alternatively, for example, TDCC can also be measured by real-time cell growth inhibition assay as described in Example 5.2. In this assay, target cells (e.g. HER2 S310F-expressing cells) are incubated with T cells (e.g. PBMCs) or expanded T cells in the presence of a test antibody on a 96-well plate, and the growth of the target cells is monitored by methods known in the art, for example, by using a suitable analyzing instrument (e.g. xCELLigence Real-Time Cell Analyzer). The rate of cell growth inhibition (CGI: %) is determined from the cell index value according to the formulation given as CGI (%)=100−(CIAb×100/CINoAb). “CIAb” represents the cell index value of wells with the antibody on a specific experimental time and “CINoAb” represents the average cell index value of wells without the antibody. If the CGI rate of the antibody is high, i.e., has a significantly positive value, it can be said that the antibody has TDCC activity and is more preferable in the present invention.

In a preferred aspect, an antibody (or antigen-binding molecule) of the present invention has T cell activation activity. T cell activation can be assayed by methods known in the art, such as a method using an engineered T cell line that expresses a reporter gene (e.g. luciferase) in response to its activation (e.g. Jurkat/NFAT-RE Reporter Cell Line (T Cell Activation Bioassay, Promega)). In this method, target cells (e.g. HER2 S310F-expressing cells) are cultured with T cells in the presence of a test antibody, and then the level or activity of the expression product of the reporter gene is measured by appropriate methods as an index of T cell activation. When the reporter gene is a luciferase gene, luminescence arising from reaction between luciferase and its substrate may be measured as an index of T cell activation. If T cell activation measured as described above is higher, the test antibody is determined to have higher T cell activation activity.

In one preferred embodiment, the multispecific antigen-binding molecule of the present invention comprises one or more polypeptide chains as listed in Table 3.

### Chimeric Antigen Receptor (CAR)

Another aspect of the invention relates to immune cells that express a HER2 S310F-targeting chimeric antigen receptor (CAR) and uses thereof. In some embodiments, a CAR of the present invention comprises the first antigen binding domain of the present invention which specifically bind to HER2 S310F, preferably one of the antigen binding domains as described in Table 2. In certain aspects, the CAR comprises a scFv specific for HER2 S310F, or any natural or artificial moiety that can specifically bind HER2 S310F. In particular embodiments, the CAR utilizes a single chain variable fragment (scFv) specific for HER2 S310F. In certain embodiments, the CAR utilizes an scFv specific for HER2 S310F that is derived from a monoclonal antibody. The CAR may be a second generation or third generation CAR, but in specific embodiments the CAR is not a third generation CAR and comprises only one costimulatory endodomain.

In specific embodiments, an individual is given a certain type of immunotherapy for treating and preventing HER2 S310F-positive cancer, such as an immune cell that recognizes HER2 S310F. In specific embodiments, the immune cell is a T cell, NK cell, or NKT cell. Other effector cells include those that can exhibit antitumor activities either innately or are modified to exhibit this effect. In specific embodiments the immune cells comprise HER2 S310F-specific CAR and may be further modified other than the HER2 S310F-specific CAR. In specific embodiments, the HER2 S310F-specific CAR comprises a scFv derived from any of the antigen-binding molecules described in Table 2. Another genetic modification of the immune cells is to express one or more chemokine receptors, such that they are utilized to enhance T-cell homing to tumor sites, for example. In specific embodiments of the CAR T cells, one can transgenically express one or more stimulatory cytokines. In certain embodiments, one can render HER2 S310F-CAR T cells resistant to an inhibitory tumor microenvironment. One may also avoid ‘on target/off cancer’ toxicity with genetic modifications to increase safety, such as an inducible suicide gene (such as caspase-9, for example) and/or inhibitory receptors to limit the effector function of T cells to tumor sites.

In particular embodiments, an individual in need thereof, such as one that is known to have a HER2 S310F-positive cancer or suspected of having a HER2 S310F-positive cancer, is provided a therapeutically effective amount of immune cells encompassed by the disclosure. In particular embodiments, an individual may be given between 1×104/m2 and 1×1010/m2 HER2 S310F-CAR T cells in a given administration, although other doses may be utilized. Multiple administrations of cells may be provided to the individual. In certain embodiments, one does or does not use lymphodepleting chemotherapy or irradiation prior to T-cell transfer. In particular embodiments, there is no post-therapy infusion with a cytokine, such as IL2, although in alternative embodiments there is post-therapy infusion with a cytokine. In specific embodiments, one can combine HER2 S310F-CAR immune cells with one or more additional immunological cancer therapies, such as checkpoint antibodies, immune modulating agents, or vaccines to increase T-cell activation and prolong in vivo survival. Other cancer therapies may also be used, such as surgery, radiation, drug therapy, and/or hormone therapy, for example.

In one embodiment, there is a polynucleotide that encodes a HER2 S310F chimeric antigen receptor, and the chimeric antigen receptor may comprise a transmembrane domain selected from the group consisting of CD3-zeta, CD28. CD8, 4-1BB, CTLA4, CD27, and a combination thereof. In some embodiments, the chimeric antigen receptor comprises no more than one costimulatory endodomain, although in certain embodiments the chimeric antigen receptor comprises more than one costimulatory endodomain. In particular embodiments, the chimeric antigen receptor comprises co-stimulatory molecule endodomains selected from the group consisting of CD28, CD27, 4-1BB, OX40 ICOS, Myd88, CD40, and a combination thereof. The chimeric antigen receptor may comprise a scFv specific for HER2 S310F that is selected from the group consisting of trastuzmab, FRP5, scFv800E6, F5cys, pertuzumab and a combination thereof.

In a certain embodiment, there is an expression vector comprising a polynucleotide encompassed by the disclosure, and the vector may be a viral vector, such as a retroviral vector, lentiviral vector, adenoviral vector, or adeno-associated viral vector, or it may be a non-viral vector.

In a particular embodiment, there is a cell comprising a polynucleotide or expression vector as encompassed by the disclosure. In specific embodiments, the cell is an immune cell, such as a T cell, NK cell, or NKT cell. The cell may be specific for another antigen, including a tumor antigen in some cases. In specific embodiments, the cells are pp65CMV-specific T cells, CMV-specific T cells, EBV-specific T cells, Varicella Virus-specific T cells, Influenza Virus-specific T cells and/or Adenovirus-specific T cells.

In one embodiment, there is a method of treating an individual for cancer, comprising the step of providing to the individual a therapeutically effective amount of a plurality of any of the cells as encompassed by the disclosure. In specific embodiments, the cancer is HER2 S310F positive. The cancer may be refractory or recurrent. In specific embodiments, the cancer is sarcoma or glioblastoma. The sarcoma may be osteosarcoma, for example. Doses may be formulated otherwise, such as per weight or per age. In certain embodiments, the therapeutically effective amount of a plurality of the cells is at a dose of at least 1×104/m2, 1×105/m2, 1×106/m2, 1×107/m2, 1×108/m2, 1×109/m2, or 1×1010/m2. In specific embodiments, the therapeutically effective amount of a plurality of the cells is at a dose of no more than 1×1010/m2, 1×109/m2, 1×108/m2, 1×107/m2, 1×106/m2, 1×105/m2, or 1×104/m2. In particular embodiments, the method occurs without or with the administration of one or more cytokines and without or with lymphodepleting therapy and occurs with a cell dose in the range of 1×104/m2 to 1×1010/m2. The cytokine may be IL2, IL7, IL12, and/or IL15.

In particular embodiments, the use of immune cells expressing HER2 S310F-specific chimeric antigen receptors occurs ex vivo. For example, the immune cells may be exposed ex vivo to one or more cells, one or more tissues and/or one or more organs for the cells to target HER2 S310F-bearing cells, including HER2 S310F-expressing cancer cells, In a specific embodiment, the HER2 S310F-specific chimeric antigen receptor-expressing immune cells are utilized to process one or more cells, one or more tissues and/or one or more organs ex vivo. In particular examples, one can purge tissue(s) or organ(s) from some or all HER2 S310F-expressing cancer cells by exposing ex vivo an effective amount of the HER2 S310F-specific chimeric antigen receptor-expressing immune cells to the respective tissue(s) or organ(s). As one example, bone marrow can be exposed ex vivo to the HER2 S310F-specific chimeric antigen receptor-expressing immune cells prior to transplant or HER2 S310F-specific chimeric antigen receptor-expressing immune cells can be used for processing an organ that harbors a malignancy prior to introduction into a host in need thereof.

Methods of generating immune cells that express a HER2 S310F-specific chimeric antigen receptor are contemplated herein. In specific embodiments, immune cells to be manipulated to express a HER2 S310F-specific chimeric antigen receptor are obtained from another party, including commercially or a skilled artisan, or are isolated from an individual to be treated with the HER2 S310F-specific chimeric antigen receptor immune cell or are isolated from another individual. The immune cell may modified to express the HER2 S310F-specific chimeric antigen receptor using standard means in the art, such as upon transduction of a polynucleotide that encodes the HER2 S310F-specific chimeric antigen receptor. In cases wherein the obtained or isolated immune cell is genetically modified to express the HER2 S310F-specific chimeric antigen receptor and also comprises a second genetic modification (such as expresses another chimeric antigen receptor or another type of non-natural receptor), the order in which the genetic modifications can occur may be in any order. Any polynucleotide that encodes a HER2 S310F-specific chimeric antigen receptor or another type of receptor may be transduced into the cell using a vector, such as a viral or non-viral vector. A viral vector may be a retroviral, lentiviral, adenoviral, adeno-associated viral vector, and so forth.

In specific embodiments, a cell is an immune cell that transgenically expresses one or more chemokine receptors, such as wherein the chemokine receptor is a receptor for a chemokine expressed by the cancer. In specific embodiments, the chemokine is CXCL1, CXCL8, CCL2, and/or CCL17. An individual may be provided a therapeutically effective amount of an additional cancer therapy, such as one given to the individual before, during, and/or after the individual is given the plurality of cells. In specific embodiments, the additional therapy comprises surgery, drug therapy, chemotherapy, radiation, immunotherapy, or a combination thereof. In specific embodiments, the individual is given lymphodepleting therapy prior to being given the plurality of cells, although in some embodiments the individual is not given lymphodepleting therapy prior to being given the plurality of cells.

In certain embodiments, the immunotherapy comprises one or more checkpoint antibodies, such as checkpoint antibodies that recognize CTLA4, PD-1, PD-L1, TIM3, BLTA, VISTA and/or LAG3. In particular embodiments, the cell comprises an inhibitory receptor.

In an embodiment, there is a kit, comprising a polynucleotide as encompassed by the disclosure, an expression vector as encompassed by the disclosure, and/or cells as encompassed by the disclosure, wherein the polynucleotide, expression vector, and or cells are housed in a suitable container.

In certain embodiments, there are HER2 S310F chimeric antigen receptor modified CMV-specific T-cells for use in cancer.

The invention further relate to methods for treating a patient for an illness including administering to the patient an effective amount of the engineered cells of the present disclosure. Various illnesses can be treated according to the present methods including cancer. In some embodiments, the cancer or cancer cell is bladder cancer, cervical cancer, colorectal cancer (CRC), non-small-cell lung carcinoma (NSCLC), esophagus cancer, head and neck cancer, skin cancer (melanoma), bile duct cancer, kidney cancer, stomach cancer, small intestine cancer, liver cancer, uterus cancer, duodenum cancer, breast cancer, gall bladder cancer, preferably characterized by expression of HER2 S310F. In one preferred embodiment, the cancer or cancer cell is bladder cancer with HER2 S310F.

In some embodiments, the method includes administering to a human patient a pharmaceutical composition including an effective antitumor amount of a population of human T cells, wherein the human T cells of the population include human T cells that comprise the nucleic acid sequence as described in the present disclosure.

### Cancer

The terms “cancer” and “cancerous” refer to or describe the physiological condition in mammals that is typically characterized by unregulated cell growth/proliferation. Examples of cancer include, but are not limited to, carcinoma, lymphoma (e.g., Hodgkin's and non-Hodgkin's lymphoma), blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma, leukemia and other lymphoproliferative disorders, and various types of head and neck cancer.

A “HER2 S310F” positive cancer comprises cancer cells characterized by HER2 S310F gene, mRNA or protein expression, which can, for example, be identified by next generation sequencing (NGS) or real-time polymerase chain reaction (RT-PCR) or any known protein detection method or assay known in the art (e.g. ELISA). In some embodiments, the cancer or cancer cell is bladder cancer, cervical cancer, colorectal cancer (CRC), non-small-cell lung carcinoma (NSCLC), esophagus cancer, head and neck cancer, skin cancer (melanoma), bile duct cancer, kidney cancer, stomach cancer, small intestine cancer, liver cancer, uterus cancer, duodenum cancer, breast cancer, gall bladder cancer, preferably characterized by expression of HER2 S310F (e.g. Nat Med. 2017 June; 23(6):703-713). In one preferred embodiment, the cancer or cancer cell is bladder cancer with HER2 S310F.

### Tumor

The term “tumor” refers to all neoplastic cell growth and proliferation, whether malignant or benign, and all pre-cancerous and cancerous cells and tissues. The terms “cancer,” “cancerous,” “cell proliferative disorder,” “proliferative disorder” and “tumor” are not mutually exclusive as referred to herein.

### Ovarian Cancer

“Ovarian cancer” refers to a heterogeneous group of malignant tumors derived from the ovary. Approximately 90% of malignant ovarian tumors are epithelial in origin; the remainder are germ cell and stromal tumors. Epithelial ovarian tumors are classified into the following histological subtypes: serous adenocarcinomas (constituting about 50% of epithelial ovarian tumors); endometrioid adenocarcinomas (approximately 20%); mucinous adenocarcinomas (approximately 10%); clear cell carcinomas (approximately 5-10%); Brenner (transitional cell) tumors (relatively uncommon). The prognosis for ovarian cancer, which is the sixth most common cancer in women, is usually poor, with five year survival rates ranging from 5-30%. For reviews of ovarian cancer, see Fox et al. (2002) “Pathology of epithelial ovarian cancer,” in Ovarian Cancer ch. 9 (Jacobs et al., eds., Oxford University Press, New York); Morin et al. (2001) “Ovarian Cancer,” in Encyclopedic Reference of Cancer, pp. 654-656 (Schwab, ed., Springer-Verlag, New York). The present invention contemplates methods of diagnosing or treating any of the epithelial ovarian tumor subtypes described above, and in particular, the serous adenocarcinoma subtype.

### Gastric Cancer

The term “gastric cancer”, or “gastric tumor”, or “stomach tumor”, or “stomach cancer” refers to any tumor or cancer of the stomach, including, e.g., adenocarcinomas (such as diffuse type and intestinal type), and less prevalent forms such as lymphomas, leiomyosarcomas, and squamous cell carcinomas.

### Breast Tumor

The term “breast tumor” or “breast cancer” refers to any tumor or cancer of the breast, including, e.g., adenocarcinomas, such as invasive or in situ ductal carcinoma, invasive or in situ lobular carcinoma, medullary carcinoma, colloid carcinoma, and papillary carcinoma; and less prevalent forms, such as cystosarcoma phylloides, sarcomas, squamous cell carcinomas, and carcinosarcomas.

### Colorectal Tumor

The term “colorectal tumor” or “colorectal cancer” refers to any tumor or cancer of the large bowel, which includes the colon (the large intestine from the cecum to the rectum) and the rectum, including, e.g., adenocarcinomas and less prevalent forms, such as lymphomas and squamous cell carcinomas.

### Stomach Tumor

The term “stomach tumor” or “stomach cancer” refers to any tumor or cancer of the stomach, including, e.g., adenocarcinomas (such as diffuse type and intestinal type), and less prevalent forms such as lymphomas, leiomyosarcomas, and squamous cell carcinomas.

### Complement Dependent Cytotoxicity

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass), which are bound to their cognate antigen. To assess complement activation, a CDC assay, e.g., as described in Gazzano-Santoro et al., J. Immunol. Methods 202:163 (1996), may be performed. Polypeptide variants with altered Fc region amino acid sequences (polypeptides with a variant Fc region) and increased or decreased C1q binding capability are described, e.g., in U.S. Pat. No. 6,194,551 B1 and WO 1999/51642. See also, e.g., Idusogie et al. J. Immunol. 164: 4178-4184 (2000).

### Immunoconjugates

The invention also provides immunoconjugates comprising the antigen-binding molecule of the present invention (e.g. anti-HER2 S310F antibody) herein conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes.

In one embodiment, an immunoconjugate is an antibody-drug conjugate (ADC) in which an antibody is conjugated to one or more drugs, including but not limited to a maytansinoid (see U.S. Pat. Nos. 5,208,020, 5,416,064 and European Patent EP 0 425 235 B1); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Pat. Nos. 5,712,374, 5,714,586, 5,739,116, 5,767,285, 5,770,701, 5,770,710, 5,773,001, and 5,877,296; Hinman et al., Cancer Res. 53:3336-3342 (1993); and Lode et al., Cancer Res. 58:2925-2928 (1998)); an anthracycline such as daunomycin or doxorubicin (see Kratz et al., Current Med. Chem. 13:477-523 (2006); Jeffrey et al., Bioorganic & Med. Chem. Letters 16:358-362 (2006); Torgov et al., Bioconj. Chem. 16:717-721 (2005); Nagy et al., Proc. Natl. Acad. Sci. USA 97:829-834 (2000); Dubowchik et al., Bioorg. & Med. Chem. Letters 12:1529-1532 (2002); King et al., J. Med. Chem. 45:4336-4343 (2002); and U.S. Pat. No. 6,630,579); methotrexate; vindesine; a taxane such as docetaxel, paclitaxel, larotaxel, tesetaxel, and ortataxel; a trichothecene; and CC1065.

In another embodiment, an immunoconjugate comprises an antibody as described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolacca americana proteins (PAPI, PAPII, and PAP-S), Momordica charantia inhibitor, curcin, crotin, Saponaria officinalis inhibitor, gelonin, mitogellin, restrictocin, phenomycin, enomycin, and the tricothecenes.

In another embodiment, an immunoconjugate comprises an antibody as described herein conjugated to a radioactive atom to form a radioconjugate. A variety of radioactive isotopes are available for the production of radioconjugates. Examples include 211At, 131I, 125I, 90Y, 186Re, 188Re, 153Sm, 212Bi, 32P, 212Pb and radioactive isotopes of Lu. When the radioconjugate is used for detection, it may comprise a radioactive atom for scintigraphic studies, for example Tc-99m or 1231, or a spin label for nuclear magnetic resonance (NMR) imaging (also known as magnetic resonance imaging, MRI), such as iodine-123 again, iodine-131, indium-111, fluorine-19, carbon-13, nitrogen-15, oxygen-17, gadolinium, manganese or iron.

Conjugates of an antibody and cytotoxic agent may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionuclide to the antibody. See WO94/11026. The linker may be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52:127-131 (1992); U.S. Pat. No. 5,208,020) may be used.

The immunuoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, IL., U.S.A).

### Pharmaceutical Formulation

The term “pharmaceutical formulation” refers to a preparation which is in such form as to permit the biological activity of an active ingredient contained therein to be effective, and which contains no additional components which are unacceptably toxic to a subject to which the formulation would be administered.

### Pharmaceutically Acceptable Carrier

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

### Treatment

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or to slow the progression of a disease.

In one aspect, the invention is based, in part, on multispecific antigen-binding molecules that comprise a first domain comprising a first antibody variable region that binds to HER2 S310F, and a second domain comprising a second antibody variable region that binds to T-cell receptor complex, and use thereof. Antigen-binding molecules and antibodies of the invention are useful, e.g., for the diagnosis or treatment of cancer, especially ovarian tumor, non-small cell lung cancer, gastric cancer, and liver cancer.

### Pharmaceutical Composition

A pharmaceutical composition of the present invention, a therapeutic agent for inducing cellular cytotoxicity, a cell growth-suppressing agent, or an anticancer agent of the present invention may be formulated with different types of multispecific antigen-binding molecules, if needed. For example, the cytotoxic action against cells expressing an antigen can be enhanced by a cocktail of multiple multispecific antigen-binding molecules of the present invention.

If necessary, the multispecific antigen-binding molecules of the present invention may be encapsulated in microcapsules (microcapsules made from hydroxymethylcellulose, gelatin, poly[methylmethacrylate], and the like), and made into components of colloidal drug delivery systems (liposomes, albumin microspheres, microemulsions, nano-particles, and nano-capsules) (for example, see “Remington's Pharmaceutical Science 16th edition”, Oslo Ed. (1980)). Moreover, methods for preparing agents as sustained-release agents are known, and these can be applied to the multispecific antigen-binding molecules of the present invention (J. Biomed. Mater. Res. (1981) 15, 267-277; Chemtech. (1982) 12, 98-105; U.S. Pat. No. 3,773,719; European Patent Application (EP) Nos. EP58481 and EP133988; Biopolymers (1983) 22, 547-556).

The pharmaceutical compositions, cell growth-suppressing agents, or anticancer agents of the present invention may be administered either orally or parenterally to patients. Parental administration is preferred. Specifically, such administration methods include injection, nasal administration, transpulmonary administration, and percutaneous administration. Injections include, for example, intravenous injections, intramuscular injections, intraperitoneal injections, and subcutaneous injections. For example, pharmaceutical compositions, therapeutic agents for inducing cellular cytotoxicity, cell growth-suppressing agents, or anticancer agents of the present invention can be administered locally or systemically by injection. Furthermore, appropriate administration methods can be selected according to the patient's age and symptoms. The administered dose can be selected, for example, from the range of 0.0001 mg to 1,000 mg per kg of body weight for each administration. Alternatively, the dose can be selected, for example, from the range of 0.001 mg/body to 100,000 mg/body per patient. However, the dose of a pharmaceutical composition of the present invention is not limited to these doses.

The pharmaceutical compositions of the present invention can be formulated according to conventional methods (for example, Remington's Pharmaceutical Science, latest edition, Mark Publishing Company, Easton, U.S.A.), and may also contain pharmaceutically acceptable carriers and additives. Examples include, but are not limited to, surfactants, excipients, coloring agents, flavoring agents, preservatives, stabilizers, buffers, suspension agents, isotonic agents, binders, disintegrants, lubricants, fluidity promoting agents, and corrigents, and other commonly used carriers can be suitably used. Specific examples of the carriers include light anhydrous silicic acid, lactose, crystalline cellulose, mannitol, starch, carmellose calcium, carmellose sodium, hydroxypropyl cellulose, hydroxypropyl methylcellulose, polyvinylacetal diethylaminoacetate, polyvinylpyrrolidone, gelatin, medium-chain triglyceride, polyoxyethylene hardened castor oil 60, saccharose, carboxymethyl cellulose, corn starch, inorganic salt, and such.

Preferably, a pharmaceutical composition of the present invention comprises a multispecific antigen-binding molecule of the invention. In one aspect, the composition is a pharmaceutical composition for use in inducing cellular cytotoxicity. In another aspect, the composition is a pharmaceutical composition for use in treating or preventing cancer. The pharmaceutical composition of the present invention can be used for treating or preventing cancer. Thus, the present invention provides a method for treating or preventing cancer, in which the multispecific antigen-binding molecule of the present invention is administered to a patient in need thereof. In preferred embodiments, the cancer is bladder cancer, cervical cancer, colorectal cancer (CRC), non-small-cell lung carcinoma (NSCLC), esophagus cancer, head and neck cancer, skin cancer (melanoma), bile duct cancer, kidney cancer, stomach cancer, small intestine cancer, liver cancer, uterus cancer, duodenum cancer, breast cancer, gall bladder cancer, preferably characterized by expression of HER2 S310F. In one preferred embodiment, the cancer is bladder cancer with HER2 S310F.

### Therapeutic Methods and Compositions

Any of the antigen-binding molecules (e.g. anti-HER2 S310F antibody, anti-HER2 S310F x CD3 bispecific antibody) provided herein may be used in therapeutic methods.

In one aspect, an antigen-binding molecule of the present invention (e.g. anti-HER2 S310F antibody, anti-HER2 S310F x CD3 bispecific antibody) for use as a medicament is provided. In further aspects, an antigen-binding molecule for use in treating cancer is provided. In certain embodiments, an antigen-binding molecule for use in a method of treatment is provided. In certain embodiments, the invention provides an antigen-binding molecule for use in a method of treating an individual having cancer comprising administering to the individual an effective amount of the antigen-binding molecule. In one such embodiment, the method further comprises administering to the individual an effective amount of at least one additional therapeutic agent, e.g., as described below. In further embodiments, the invention provides an antigen-binding molecule for use in e.g. prevents ligand-independent dimerization of HER2 S310F, inhibiting proliferation and/or growth of HER2 S310F expressing cancer cell. An “individual” according to any of the above embodiments is preferably a human. In one embodiment, the cancer or cancer cell is characterized by expression of HER2 S310F. In another embodiment, the cancer or cancer cell is bladder cancer, cervical cancer, colorectal cancer (CRC), non-small-cell lung carcinoma (NSCLC), esophagus cancer, head and neck cancer, skin cancer (melanoma), bile duct cancer, kidney cancer, stomach cancer, small intestine cancer, liver cancer, uterus cancer, duodenum cancer, breast cancer, gall bladder cancer, preferably characterized by expression of HER2 S310F. In one preferred embodiment, the cancer or cancer cell is bladder cancer with HER2 S310F.

In a further aspect, the invention provides for the use of the antigen-binding molecules (e.g. anti-HER2 S310F antibody, anti-HER2 S310F x CD3 bispecific antibody) in the manufacture or preparation of a medicament. In one embodiment, the medicament is for treatment of cancer. In a further embodiment, the medicament is for use in a method of treating cancer comprising administering to an individual having cancer an effective amount of the medicament. In one such embodiment, the method further comprises administering to the individual an effective amount of at least one additional therapeutic agent, e.g., as described below. In a further embodiment, the medicament is for e.g. prevents ligand-independent dimerization of HER2 S310F, inhibiting proliferation and/or growth of HER2 S310F expressing cancer cell. In a further embodiment, the medicament is for use in a method of e.g. prevents ligand-independent dimerization of HER2 S310F, inhibiting proliferation and/or growth of HER2 S310F expressing cancer cell in an individual comprising administering to the individual an amount effective of the medicament to prevents ligand-independent dimerization of HER2 S310F, inhibiting proliferation and/or growth of HER2 S310F expressing cancer cell. An “individual” according to any of the above embodiments may be a human. In one embodiment, the cancer or cancer cell is characterized by expression of HER2 S310F. In another embodiment, the cancer or cancer cell is bladder cancer, cervical cancer, colorectal cancer (CRC), non-small-cell lung carcinoma (NSCLC), esophagus cancer, head and neck cancer, skin cancer (melanoma), bile duct cancer, kidney cancer, stomach cancer, small intestine cancer, liver cancer, uterus cancer, duodenum cancer, breast cancer, gall bladder cancer, preferably characterized by expression of HER2 S310F. In one preferred embodiment, the cancer or cancer cell is bladder cancer with HER2 S310F.

In a further aspect, the invention provides a method for treating a cancer. In one embodiment, the method comprises administering to an individual having such cancer an effective amount of an antigen-binding molecule of the invention (e.g. anti-HER2 S310F antibody, anti-HER2 S310F x CD3 bispecific antibody). In one such embodiment, the method further comprises administering to the individual an effective amount of at least one additional therapeutic agent, as described below. An “individual” according to any of the above embodiments may be a human. In one embodiment, the cancer or cancer cell is characterized by expression of HER2 S310F. In another embodiment, the cancer or cancer cell is bladder cancer, cervical cancer, colorectal cancer (CRC), non-small-cell lung carcinoma (NSCLC), esophagus cancer, head and neck cancer, skin cancer (melanoma), bile duct cancer, kidney cancer, stomach cancer, small intestine cancer, liver cancer, uterus cancer, duodenum cancer, breast cancer, gall bladder cancer, preferably characterized by expression of HER2 S310F. In one preferred embodiment, the cancer or cancer cell is bladder cancer with HER2 S310F.

In a further aspect, the invention provides pharmaceutical formulations comprising any of antigen-binding molecules of the invention (e.g. anti-HER2 S310F antibody, anti-HER2 S310F x CD3 bispecific antibody) provided herein, e.g., for use in any of the above therapeutic methods. In one embodiment, a pharmaceutical formulation comprises any of the antigen-binding molecule of the invention (e.g. anti-HER2 S310F antibody, anti-HER2 S310F x CD3 bispecific antibody) provided herein and a pharmaceutically acceptable carrier. In another embodiment, a pharmaceutical formulation comprises any of the antigen-binding molecule of the invention (e.g. anti-HER2 S310F antibody, anti-HER2 S310F x CD3 bispecific antibody) provided herein and at least one additional therapeutic agent, e.g., as described below.

### Combination Therapy

The antigen-binding molecule of the invention (e.g. anti-HER2 S310F antibody, anti-HER2 S310F x CD3 bispecific antibody) can be used either alone or in combination with other agents in a therapy. For instance, an antibody of the invention may be co-administered with at least one additional therapeutic agent. In certain embodiments, an additional therapeutic agent is an additional other cytotoxic, chemotherapeutic or anti-cancer agents, or compounds that enhance the effects of anti-HER2 S310F therapy in a patient. Such agents include, for example: any of the anti-HER2 antibodies or inhibitors that are known in the art such as trastuzumab or pertuzumab, ado-trastuzumab emtansine, lapatinib or neratinib; alkylating agents or agents with an alkylating action, such as cyclophosphamide (CTX; e.g. CYTOXAN®), chlorambucil (CHL; e.g. LEUKERAN®), cisplatin (CisP; e.g. PLATINOL®) busulfan (e.g. MYLERAN®), melphalan, carmustine (BCNU), streptozotocin, triethylenemelamine (TEM), mitomycin C, and the like; immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines;
anti-metabolites, such as methotrexate (MTX), etoposide (VP16; e.g. VEPESID®), 6-mercaptopurine (6 MP), 6-thioguanine (6TG), cytarabine (Ara-C), 5-fluorouracil (5-FU), capecitabine (e.g. XELODA®), dacarbazine (DTIC), and the like; antibiotics, such as actinomycin D, doxorubicin (DXR; e.g. ADRIAMYCIN®), daunorubicin (daunomycin), bleomycin, mithramycin and the like; alkaloids, such as vinca alkaloids such as vincristine (VCR), vinblastine, and the like; and other antitumor agents, such as paclitaxel (e.g. TAXOL®) and paclitaxel derivatives, the cytostatic agents, glucocorticoids such as dexamethasone (DEX; e.g. DECADRON®) and corticosteroids such as prednisone, nucleoside enzyme inhibitors such as hydroxyurea, amino acid depleting enzymes such as asparaginase, leucovorin and other folic acid derivatives, and similar, diverse antitumor agents.

The following agents may also be used as additional agents: arnifostine (e.g. ETHYOL®) dactinomycin, mechlorethamine (nitrogen mustard), streptozocin, cyclophosphamide, lomustine (CCNU), doxorubicin lipo (e.g. DOXIL®), gemcitabine (e.g. GEMZAR®), daunorubicin lipo (e.g. DAUNOXOME®, procarbazine, mitomycin, docetaxel (e.g. TAXOTERE®), aldesleukin, carboplatin, oxaliplatin, cladribine, camptothecin, CPT 11 (irinotecan), 10-hydroxy 7-ethyl-camptothecin (SN38), floxuridine, fludarabine, ifosfamide, idarubicin, mesna, interferon beta, interferon alpha, mitoxantrone, topotecan, leuprolide, megestrol, melphalan, mercaptopurine, plicamycin, mitotane, pegaspargase, pentostatin, pipobroman, plicamycin, tamoxifen, teniposide, testolactone, thioguanine, thiotepa, uracil mustard, vinorelbine, chlorambucil.

In one embodiment, an anti-hormonal agent may be also used in combination with the antigen-binding molecule of the invention for treatment of HER2 S310F positive cancer or metastasis of HER2 S310F positive cancer in a patient. As used herein, the term “anti-hormonal agent” includes natural or synthetic organic or peptidic compounds that act to regulate or inhibit hormone action on tumors. Antihormonal agents include, for example: steroid receptor antagonists, anti-estrogens such as tamoxifen, raloxifene, aromatase inhibiting 4(5)-imidazoles, other aromatase inhibitors, 42-hydroxytamoxifen, trioxifene, keoxifene, LY 117018, onapristone, and toremifene (e.g. FARESTON®); anti-androgens such as flutamide, nilutamide, bicalutamide, leuprolide, and goserelin; and pharmaceutically acceptable salts, acids or derivatives of any of the above; agonists and/or antagonists of glycoprotein hormones such as follicle stimulating hormone (FSH), thyroid stimulating hormone (TSH), and luteinizing hormone (LH) and LHRH (luteinizing hormone-releasing hormone); the LHRH agonist goserelin acetate, commercially available as ZOLADEX®) (AstraZeneca); the LHRH antagonist D-alaninamide N-acetyl-3-(2-naphthalenyl)-D-alanyl-4-chloro-D-phenylalanyl-3-(3-pyridinyl)-D-alanyl-L-seryl-N-6-(3-pyridinylcarbonyl)-L-lysyl-N-6-(3-pyridinyl-carbonyl)-D-lysyl-L-leucyl-N-6-(1-methylethyl)-L-lysyl-L-proline (e.g. ANTIDE®, Ares-Serono); the LHRH antagonist ganirelix acetate; the steroidal anti-androgens cyproterone acetate (CPA) and megestrol acetate, commercially available as MEGACE®) (Bristol-Myers Oncology); the nonsteroidal anti-androgen flutamide(2-methyl-N-[4,20-nitro-3-(trifluoromethyl)phenylpropanamide), commercially available as EULEXIN®) (Schering Corp.); the non-steroidal anti-androgen nilutamide, (5,5-dimethyl-3-[4-nitro-3-(trifluoromethyl-4′-nitrophenyl)-4,4-dimethyl-imidazolidine-dione); and antagonists for other non-permissive receptors, such as antagonists for RAR (retinoic acid receptor), RXR (retinoid X receptor), TR (thyroid receptor), VDR (vitamin-D receptor), and the like.

Such combination therapies noted above encompass combined administration (where two or more therapeutic agents are included in the same or separate formulations), and separate administration, in which case, administration of the antibody of the invention can occur prior to, simultaneously, and/or following, administration of the additional therapeutic agent or agents. In one embodiment, administration of the antigen-binding molecule and administration of an additional therapeutic agent occur within about one month, or within about one, two or three weeks, or within about one, two, three, four, five, or six days, of each other. The antigen-binding molecule of the invention (e.g. anti-HER2 S310F antibody, anti-HER2 S310F x CD3 bispecific antibody) can also be used in combination with radiation therapy.

The antigen-binding molecule of the invention (e.g. anti-HER2 S310F antibody, anti-HER2 S310F x CD3 bispecific antibody) (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

The antigen-binding molecule of the invention (e.g. anti-HER2 S310F antibody, anti-HER2 S310F x CD3 bispecific antibody) would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antigen-binding molecule need not be, but is optionally formulated with one or more agents currently used to prevent or treat the disorder in question. The effective amount of such other agents depends on the amount of antigen-binding molecule present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

It is understood that any of the above formulations or therapeutic methods may be carried out using an immunoconjugate of the invention in place of or in addition to an antigen-binding molecule of the invention.

The present invention also provides methods for damaging cells expressing HER2 S310F or for suppressing the cell growth by contacting the cells expressing HER2 S310F with a monospecific or multispecific antigen-binding molecule of the present invention that binds to HER2 S310F (e.g. anti-HER2 S310F antibody, anti-HER2 S310F x CD3 bispecific antibody). Monoclonal antibodies that bind to HER2 S310F are described above as a monospecific or multispecific antigen-binding molecule of the present invention, which is included in the therapeutic agents for inducing cellular cytotoxicity, cell growth-suppressing agents, and anticancer agents of the present invention. Cells to which a monospecific or multispecific antigen-binding molecule of the present invention binds are not particularly limited, as long as they express HER2 S310F. Specifically, in the present invention, the preferred cancer antigen-expressing cells includes a HER2 S310F-positive cancer comprises cancer cells characterized by HER2 S310F gene, mRNA or protein expression, which can, for example, be identified by next generation sequencing (NGS) or real-time polymerase chain reaction (RT-PCR) or any known protein detection method or assay known in the art (e.g. ELISA). In some embodiments, the cancer or cancer cell is bladder cancer, cervical cancer, colorectal cancer (CRC), non-small-cell lung carcinoma (NSCLC), esophagus cancer, head and neck cancer, skin cancer (melanoma), bile duct cancer, kidney cancer, stomach cancer, small intestine cancer, liver cancer, uterus cancer, duodenum cancer, breast cancer, gall bladder cancer, preferably characterized by expression of HER2 S310F (e.g. Nat Med. 2017 June; 23(6):703-713). In one preferred embodiment, the cancer or cancer cell is bladder cancer with HER2 S310F.

In the present invention, “contact” can be carried out, for example, by adding a multispecific antigen-binding molecule of the present invention to culture media of cells expressing HER2 S310F cultured in vitro. In this case, a multispecific antigen-binding molecule to be added can be used in an appropriate form, such as a solution or solid prepared by lyophilization or the like. When the multispecific antigen-binding molecule of the present invention is added as an aqueous solution, the solution may be a pure aqueous solution containing the multispecific antigen-binding molecule alone or a solution containing, for example, an above-described surfactant, excipient, coloring agent, flavoring agent, preservative, stabilizer, buffering agent, suspending agent, isotonizing agent, binder, disintegrator, lubricant, fluidity accelerator, and corrigent. The added concentration is not particularly limited; however, the final concentration in a culture medium is preferably in a range of 1 pg/ml to 1 g/ml, more preferably 1 ng/ml to 1 mg/ml, and still more preferably 1 micro g/ml to 1 mg/ml.

In another embodiment of the present invention, “contact” can also be carried out by administration to nonhuman animals transplanted with HER2 S310F-expressing cells in vivo or to animals having cancer cells expressing HER2 S310F endogenously. The administration method may be oral or parenteral. Parenteral administration is particularly preferred. Specifically, the parenteral administration method includes injection, nasal administration, pulmonary administration, and percutaneous administration. Injections include, for example, intravenous injections, intramuscular injections, intraperitoneal injections, and subcutaneous injections. For example, pharmaceutical compositions, therapeutic agents for inducing cellular cytotoxicity, cell growth-suppressing agents, or anticancer agents of the present invention can be administered locally or systemically by injection. Furthermore, an appropriate administration method can be selected according to the age and symptoms of an animal subject. When the multispecific antigen-binding molecule is administered as an aqueous solution, the solution may be a pure aqueous solution containing the multispecific antigen-binding molecule alone or a solution containing, for example, an above-described surfactant, excipient, coloring agent, flavoring agent, preservative, stabilizer, buffering agent, suspending agent, isotonizing agent, binder, disintegrator, lubricant, fluidity accelerator, and corrigent. The administered dose can be selected, for example, from the range of 0.0001 to 1,000 mg per kg of body weight for each administration. Alternatively, the dose can be selected, for example, from the range of 0.001 to 100,000 mg/body for each patient. However, the dose of a multispecific antigen-binding molecule of the present invention is not limited to these examples.

The methods described below are preferably used as a method for assessing or determining cellular cytotoxicity caused by contacting a multispecific antigen-binding molecule of the present invention with HER2 S310F-expressing cells to which the antigen-binding domain forming the multispecific antigen-binding molecules of the present invention binds. The methods for assessing or determining the cytotoxic activity in vitro include methods for determining the activity of cytotoxic T-cells or the like. Whether a multispecific antigen-binding molecule of the present invention has the activity of inducing T-cell mediated cellular cytotoxicity can be determined by known methods (see, for example, Current protocols in Immunology, Chapter 7. Immunologic studies in humans, Editor, John E, Coligan et al., John Wiley & Sons, Inc., (1993)). In the cytotoxicity assay, a multispecific antigen-binding molecule whose antigen-binding domain binds to an antigen different from HER2 S310F and which is not expressed in the cells is used as a control multispecific antigen-binding molecule. The control multispecific antigen-binding molecule is assayed in the same manner. Then, the activity is assessed by testing whether a multispecific antigen-binding molecule of the present invention exhibits a stronger cytotoxic activity than that of a control multispecific antigen-binding molecule.

Meanwhile, the in vivo anti-tumor efficacy is assessed or determined, for example, by the following procedure. Cells expressing the antigen to which the antigen-binding domain forming a multispecific antigen-binding molecule of the present invention binds are transplanted intracutaneously or subcutaneously to a nonhuman animal subject. Then, from the day of transplantation or thereafter, a test multispecific antigen-binding molecule is administered into vein or peritoneal cavity every day or at intervals of several days. The tumor size is measured over time. Difference in the change of tumor size can be defined as the cytotoxic activity. As in an in vitro assay, a control multispecific antigen-binding molecule is administered. The multispecific antigen-binding molecule of the present invention can be judged to have cytotoxic activity when the tumor size is smaller in the group administered with the multispecific antigen-binding molecule of the present invention than in the group administered with the control multispecific antigen-binding molecule.

An MTT method and measurement of isotope-labeled thymidine uptake into cells are preferably used to assess or determine the effect of contact with a multispecific antigen-binding molecule of the present invention to suppress the growth of cells expressing an antigen to which the antigen-binding domain forming the multispecific antigen-binding molecule binds. Meanwhile, the same methods described above for assessing or determining the in vivo cytotoxic activity can be used preferably to assess or determine the activity of suppressing cell growth in vivo.

The present invention also provides kits for use in a method of the present invention, which contain a multispecific antigen-binding molecule of the present invention or a multispecific antigen-binding molecule produced by a method of the present invention. The kits may be packaged with an additional pharmaceutically acceptable carrier or medium, or instruction manual describing how to use the kits, etc.

In addition, the present invention relates to multispecific antigen-binding molecules of the present invention or multispecific antigen-binding molecules produced by a method of the present invention for use in a method of the present invention.

### Methods and Compositions for Diagnostics and Detection

In certain embodiments, any of the antigen-binding molecule of the invention (e.g. anti-HER2 S310F antibody) provided herein is useful for detecting the presence of HER2 S310F in a biological sample. The term “detecting” as used herein encompasses quantitative or qualitative detection. In certain embodiments, a biological sample comprises a cell or tissue, such as bladder tissue.

In one embodiment, an anti-HER2 S310F antibody for use in a method of diagnosis or detection is provided. In a further aspect, a method of detecting the presence of HER2 S310F in a biological sample is provided. In certain embodiments, the method comprises contacting the biological sample with an anti-HER2 S310F antibody as described herein under conditions permissive for binding of the anti-HER2 S310F antibody to HER2 S310F, and detecting whether a complex is formed between the anti-HER2 S310F antibody and HER2 S310F. Such method may be an in vitro or in vivo method. In one embodiment, an anti-HER2 S310F antibody is used to select subjects eligible for therapy with an anti-HER2 S310F antibody, where HER2 S310F is a biomarker for selection of patients. In one embodiment, an anti-HER2 S310F antibody is used in a method of diagnosing cancer in an individual, or of predicting cancer treatment response in an individual, comprising determining the presence of HER2 S310F in an isolated sample from patient. In one embodiment, the cancer or cancer cell is characterized by expression of HER2 S310F. In another embodiment, the cancer or cancer cell is bladder cancer, cervical cancer, colorectal cancer (CRC), non-small-cell lung carcinoma (NSCLC), esophagus cancer, head and neck cancer, skin cancer (melanoma), bile duct cancer, kidney cancer, stomach cancer, small intestine cancer, liver cancer, uterus cancer, duodenum cancer, breast cancer, gall bladder cancer, preferably characterized by expression of HER2 S310F. In one preferred embodiment, the cancer or cancer cell is bladder cancer with HER2 S310F.

In certain embodiments, labeled anti-HER2 S310F antibodies are provided. Labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction. Exemplary labels include, but are not limited to, the radioisotopes 32P, 14C, 125I, 3H, and 131I, fluorophores such as rare earth chelates or fluorescein and its derivatives, rhodamine and its derivatives, dansyl, umbelliferone, luceriferases, e.g., firefly luciferase and bacterial luciferase (U.S. Pat. No. 4,737,456), luciferin, 2,3-dihydrophthalazinediones, horseradish peroxidase (RP), alkaline phosphatase, beta-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, those coupled with an enzyme that employs hydrogen peroxide to oxidize a dye precursor such as HRP, lactoperoxidase, or microperoxidase, biotin/avidin, spin labels, bacteriophage labels, stable free radicals, and the like.

All documents cited herein are incorporated herein by reference.

### EXAMPLES

The following are examples of methods and compositions of the invention. It is understood that various other embodiments may be practiced, given the general description provided above.

### Example 1. Expression and Purification of the Human HER2 S310F Extracellular Domain (ECD) and Wild-Type HER2 ECD

A synthesized polypeptide comprising amino acids 23-646 of human HER2 S310F ECD with rabbit Fc tag on its C terminus (SEQ ID NO: 1) was expressed transiently using the Expi293 cell line (Thermo Fisher). Conditioned media expressing the synthesized polypeptide were applied to a column packed with MabSelect SuRe resin (GE Healthcare) and eluted with Na acetate. Fractions containing the synthesized polypeptide were collected and subsequently subjected to a Superdex 200 gel filtration column (GE healthcare) equilibrated with 1×D-PBS. Fractions containing the synthesized polypeptide were then pooled and stored at −80 degrees C.

SEQ ID NO: 1 (shown below) depicts the amino acid sequence of 23-646 of human HER2 S310F ECD (the F amino acid residue corresponding to position 310 of HER2 is bold and underlined) with linker (bold) followed by a rabbit Fc tag (underlined) on its C terminus including the signal sequence (italics, first 19 amino acid residues). The signal sequence is not part of the mature polypeptide chain.

MGWSCIILFLVATATGVHSTQVCTGTDMKLRLPASPETHLDMLRHLYQGC
QVVQGNLELTYLPTNASLSFLQDIQEVQGYVLIAHNQVRQVPLQRLRIVR
GTQLFEDNYALAVLDNGDPLNNTTPVTGASPGGLRELQLRSLTEILKGGV
LIQRNPQLCYQDTILWKDIFHKNNQLALTLIDTNRSRACHPCSPMCKGSR
CWGESSEDCQSLTRTVCAGGCARCKGPLPTDCCHEQCAAGCTGPKHSDCL
ACLHFNHSGICELHCPALVTYNTDTFESMPNPEGRYTEGASCVTACPYNY
LSTDVGFCTLVCPLHNQEVTAEDGTQRCEKCSKPCARVCYGLGMEHLREV
RAVTSANIQEFAGCKKIFGSLAFLPESFDGDPASNTAPLQPEQLQVFETL
EEITGYLYISAWPDSLPDLSVFQNLQVIRGRILHNGAYSLTLQGLGISWL
GLRSLRELGSGLALIHHNTHLCFVHTVPWDQLFRNPHQALLHTANRPEDE
CVGEGLACHQLCARGHCWGPGPTQCVNCSQFLRGQECVEECRVLQGLPRE
YVNARHCLPCHPECQPQNGSVTCFGPEADQCVACAHYKDPPFCVARCPSG
VKPDLSYMPIWKFPDEEGACQPCPINCTHSCVDLDDKGCPAEQGGGGSAP
STCSKPMCPPPELLGGPSVFIFPPKPKDTLMISRTPEVTCVVVDVSQDDP
EVQFTWYINNEQVRTARPPLREQQFNSTIRVVSTLPIAHQDWLRGKEFKC
KVHNKALPAPIEKTISKARGQPLEPKVYTMGPPREELSSRSVSLTCMING
FYPSDISVEWEKNGKAEDNYKTTPTVLDSDGSYFLYSKLSVPTSEWQRGD
VFTCSVMHEALHNHYTQKSISRSPGK

A synthesized polypeptide comprising amino acids 23-646 of human HER2 S310F ECD with FLAG tag on its C terminus (SEQ ID NO: 2) was expressed transiently using the Expi293 cell line (Thermo Fisher). Conditioned media expressing the synthesized polypeptide were applied to a column packed with anti-FLAG M2 affinity resin (Sigma) and eluted with a FLAG peptide (Sigma). Fractions containing the synthesized polypeptide were collected and subsequently subjected to a Superdex 200 gel filtration column (GE healthcare) equilibrated with 1×D-PBS. Fractions containing the synthesized polypeptide were then pooled and stored at −80 degrees C.

SEQ ID NO: 2 (shown below) depicts the amino acid sequence of 23-646 of human HER2 S310F ECD (the F amino acid residue corresponding to position 310 of HER2 is bold and underlined) with linker (bold) followed by a FLAG tag (underlined) on its C terminus including the signal sequence (italics, first 19 amino acid residues). The signal sequence is not part of the mature polypeptide chain.

MGWSCIILFLVATATGVHSTQVCTGTDMKLRLPASPETHLDMLRHLYQGC
QVVQGNLELTYLPTNASLSFLQDIQEVQGYVLIAHNQVRQVPLQRLRIVR
GTQLFEDNYALAVLDNGDPLNNTTPVTGASPGGLRELQLRSLTEILKGGV
LIQRNPQLCYQDTILWKDIFHKNNQLALTLIDTNRSRACHPCSPMCKGSR
CWGESSEDCQSLTRTVCAGGCARCKGPLPTDCCHEQCAAGCTGPKHSDCL
ACLHFNHSGICELHCPALVTYNTDTFESMPNPEGRYTFGASCVTACPYNY
LSTDVGFCTLVCPLHNQEVTAEDGTQRCEKCSKPCARVCYGLGMEHLREV
RAVTSANIQEFAGCKKIFGSLAFLPESFDGDPASNTAPLQPEQLQVFETL
EEITGYLYISAWPDSLPDLSVFQNLQVIRGRILHNGAYSLTLQGLGISWL
GLRSLRELGSGLALIHHNTHLCFVHTVPWDQLFRNPHQALLHTANRPEDE
CVGEGLACHQLCARGHCWGPGPTQCVNCSQFLRGQECVEECRVLQGLPRE
YVNARHCLPCHPECQPQNGSVTCFGPEADQCVACAHYKDPPFCVARCPSG
VKPDLSYMPIWKFPDEEGACQPCPINCTHSCVDLDDKGCPAEQGGGGSDY
KDDDDK

A synthesized polypeptide comprising amino acids 23-646 of human wild-type HER2 ECD with twin Strep tag on its C terminus (SEQ ID NO: 3) was expressed transiently using the Expi293 cell line (Thermo Fisher). Conditioned media expressing the synthesized polypeptide were applied to a column packed with Strep Tactin resin (IBA Lifesciences) and eluted with Desthiobiotin (Sigma). Fractions containing the synthesized polypeptide were collected and subsequently subjected to a Superdex 200 gel filtration column (GE Healthcare) equilibrated with 1×D-PBS. Fractions containing the synthesized polypeptide were then pooled and stored at −80 degrees C.

SEQ ID NO: 3 (shown below) depicts the amino acid sequence of 23-646 of human wild-type HER2 ECD (the S amino acid residue corresponding to position 310 of HER2 is bold and underlined) with linker (bold) followed by a twin Strep tag (underlined) on its C terminus including the signal sequence (italics, first 19 aa). The signal sequence is not part of the mature polypeptide chain.

MGWSCIILFLVATATGVHSTQVCTGTDMKLRLPASPETHLDMLRHLYQG
CQVVQGNLELTYLPTNASLSFLQDIQEVQGYVLIAHNQVRQVPLQRLRIV
RGTQLFEDNYALAVLDNGDPLNNTTPVTGASPGGLRELQLRSLTEILKGG
VLIQRNPQLCYQDTILWKDIFHKNNQLALTLIDTNRSRACHPCSPMCKGS
RCWGESSEDCQSLTRTVCAGGCARCKGPLPTDCCHEQCAAGCTGPKHSDC
LACLHFNHSGICELHCPALVTYNTDTFESMPNPEGRYTFGASCVTACPYN
YLSTDVGSCTLVCPLHNQEVTAEDGTQRCEKCSKPCARVCYGLGMEHLRE
VRAVTSANIQEFAGCKKIFGSLAFLPESEDGDPASNTAPLQPEQLQVFET
LEEITGYLYISAWPDSLPDLSVFQNLQVIRGRILHNGAYSLTLQGLGISW
LGLRSLRELGSGLALIHHNTHLCFVHTVPWDQLFRNPHQALLHTANRPED
ECVGEGLACHQLCARGHCWGPGPTQCVNCSQFLRGQECVEECRVLQGLPR
EYVNARHCLPCHPECQPQNGSVTCFGPEADQCVACAHYKDPPFCVARCPS
GVKPDLSYMPIWKFPDEEGACQPCPINCTHSCVDLDDKGCPAEQGGGGSS
AWSHPQFEKGGGSGGGSGGSAWSHPQFEK

### Example 2. Establishment of FreeStyle 293 Cell Lines Expressing Human HER2 S310F or Wild-Type HER2

FreeStyle 293 cells were transfected with pcDNA3.1 (+) Hygro-HER2 S310F or pcDNA3.1 (+) Hygro-wild-type HER2 expression vectors using 293fectin and selected using hygromycin B (10 micrograms (micro g)/ml). FreeStyle 293 cells stably expressing human HER2 S310F and human wild-type HER2 were sorted by staining with anti-HER2 antibody (Biolegend, 324402) (FACS ARIA® III, BD), expanded in culture and cryopreserved for future use.

### Example 3. Generation and Screening of Anti-HER2 S310F Monospecific Antibodies

Anti-HER2 S310F antibodies were generated through both protein immunization and DNA immunization.

For Protein immunization, three NZW rabbits were first immunized intradermally (ID) with 100 micro g of recombinant human HER2 S310F ECD protein with rabbit Fc Tag. Two weeks after the initial immunization, three more weekly doses of the same immunogen were given (50 micro g/dose/rabbit). One week after the final immunization, spleen and blood from immunized rabbits were collected for B cell isolation.

For DNA immunization, three NZW rabbits were immunized with pCXND3-HER2 S310F plasmid coated gold particles (1 micro g DNA per shot for total 40 shots) by Helios Gene Gun system (Bio-Rad) according to the manufacturer's instruction (J Virol. 2013 September; 87(18):10232-43). At the same time, combination of intradermal electroporation (100 micro g DNA per site for total 4 sites at legs and shoulders) and intramuscular electroporation (200 micro g DNA per site for total 4 sites at two legs) with pCXND3-HER2 S310F plasmid were carried out as described in Vaccine. 2008 Apr. 16; 26(17):2100-10; J Pharmacol Toxicol Methods. 2008 July-August; 58(1):27-31; and Int J Pharm. 2005 Apr. 27; 294(1-2):53-63. The immunization was repeated weekly for seven times followed by blood and spleen collection.

B cells were stained with recombinant human HER2 S310F ECD protein with FLAG tag and recombinant human wild-type HER2 ECD protein with twin-strep tag. HER2 S310F ECD specific B cells were sorted with a flow cytometry (FCM) cell sorter (FACSARIA® III, BD Biosciences), and plated in 96-well plates at one cell/well density, together with 25,000 cells/well of EL4 cells (European Collection of Cell Cultures) pre-treated with mitomycin C (Sigma), in culture medium supplemented with rabbit T-cell conditioned medium as described in WO2016098356A1. After 7-12 days culturing, B-cell culture supernatants were collected for further analysis and cell pellets were cryopreserved.

In total, 6015 B cell supernatants were screened and B cell supernatants containing antibodies binding specifically to HER2 S310F were identified by flow cytometry analysis and ELISA. FreeStyle 293 cells transiently expressing human HER2 S310F and human wild-type HER2 were first stained with Violet and Far Red CellTrace dye (Invitrogen). Equal number of FreeStyle 293 overexpressingHER2 S310F cells (Violet), FreeStyle 293 overexpressing wild-type HER2 cells (Far Red) and parental FreeStyle 293 cells (unstained) were mixed together, stained with B cell supernatants and anti-rabbit IgG PE antibody, and analyzed by iQUE screener PLUS (IntelliCyt). Antibodies from 20 B cell supernatants showed stronger binding to FreeStyle 293 cells overexpressing HER2 S310F than to parental FreeStyle 293 cells and FreeStyle 293 cells overexpressing wild-type HER2. At the same time, binding of antibodies in B cell supernatants to recombinant HER2 S310F ECD protein and wild-type HER2 ECD protein were evaluated by ELISA. 61 B cell lines showed stronger binding to recombinant HER2 S310F ECD protein than wild type HER2 ECD protein. Together with 3 additional controls, 84 B cells lines were selected for antibody gene cloning and further analysis (MHR0001-0084).

RNA was extracted from corresponding cell pellets by using ZR-96 Quick-RNA kits (ZYMO RESEARCH). The DNA of their heavy chain and light chain variable regions were amplified by reverse transcription PCR and cloned into expression vectors with human heavy chain constant region sequence (SEQ ID NO: 133) and expression vector containing the human light chain constant region sequence (SEQ ID NO: 136) respectively. Recombinant antibodies were expressed transiently in FreeStyle 293-F cells according to the manufacturer's instructions (Life technologies) and purified using AssayMAP Bravo platform with protein A cartridge (Agilent).

Binding of these 84 purified antibodies to HER2 S310F were checked again by flow cytometry analysis using FreeStyle 293 cells with HER2 S310F and FreeStyle 293 cells with wild-type HER2. 12 antibodies showing specific binding to HER2 S310F were selected for downstream analysis (Table 2).

TABLE 2
Anti-HER2 S310F monospecific antibodies

| | SEQ ID NO: | | | | | | | | | |
|---|---|---|---|---|---|---|---|---|---|---|
| | Heavy | Heavy | | | | Light | Light | | | |
| | chain | chain | | | | chain | chain | | | |
| Antibody | variable | constant | HVR- | HVR- | HVR- | variable | constant | HVR- | HVR- | HVR- |
| name | region | region | H1 | H2 | H3 | region | region | L1 | L2 | L3 |
| MHR0007 | 5 | 133 | 29 | 41 | 53 | 17 | 136 | 65 | 77 | 89 |
| MHR0009 | 6 | | 30 | 42 | 54 | 18 | | 66 | 78 | 90 |
| MHR0010 | 7 | | 31 | 43 | 55 | 19 | | 67 | 79 | 91 |
| MHR0012 | 8 | | 32 | 44 | 56 | 20 | | 68 | 80 | 92 |
| MHR0014 | 9 | | 33 | 45 | 57 | 21 | | 69 | 81 | 93 |
| MHR0015 | 10 | | 34 | 46 | 58 | 22 | | 70 | 82 | 94 |
| MHR0016 | 11 | | 35 | 47 | 59 | 23 | | 71 | 83 | 95 |
| MHR0017 | 12 | | 36 | 48 | 60 | 24 | | 72 | 84 | 96 |
| MHR0018 | 13 | | 37 | 49 | 61 | 25 | | 73 | 85 | 97 |
| MHR0019 | 14 | | 38 | 50 | 62 | 26 | | 74 | 86 | 98 |
| MHR0060 | 15 | | 39 | 51 | 63 | 27 | | 75 | 87 | 99 |
| MHR0060′ | 15 | | 39 | 51 | 63 | 139 | | 75 | 87 | 99 |
| MHR0082 | 16 | | 40 | 52 | 64 | 28 | | 76 | 88 | 100 |

### Example 4. Binding Analysis of the Anti-HER2 S310F Monospecific Antibodies

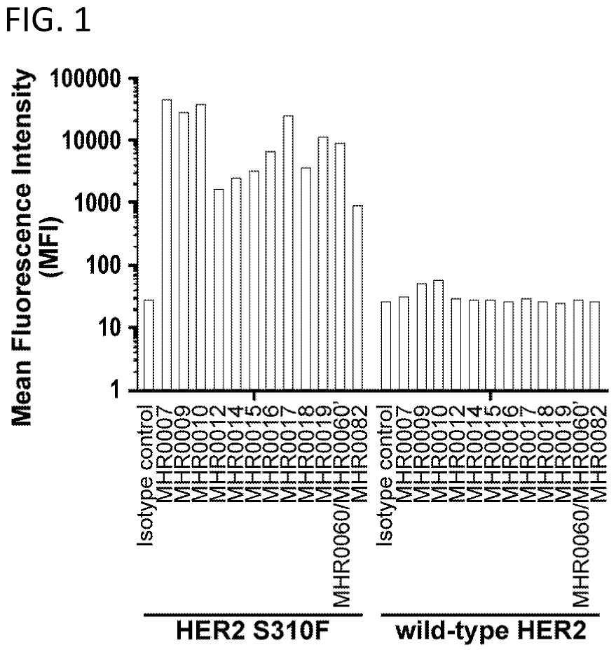

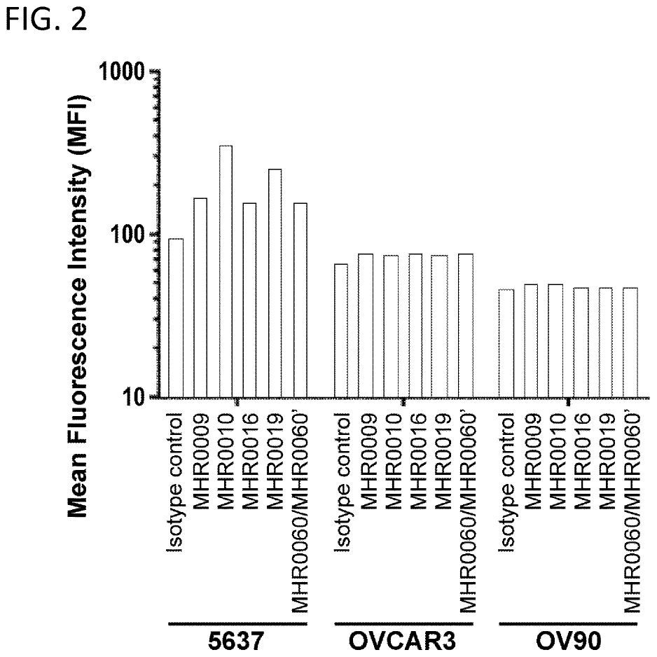

FIG. 1 shows binding of the anti-HER2 S310F antibodies to the FreeStyle 293 transfectant expressing HER2 S310F and FreeStyle 293 transfectant expressing wild-type HER2 as determined by FCM analysis. FIG. 2 shows binding of the anti-HER2 S310F antibodies to the 5637 cancer cell line with HER2 S310F (Proc Natl Acad Sci USA. 2012 Sep. 4; 109(36):14476-81), OV90 cancer cell line with wild-type HER2, and OVCAR3 cancer cell line with wild-type HER2 as determined by FCM analysis. Anti-HER2 S310F antibodies were incubated with each cell line for 30 minutes at 4 degrees C. and washed with the Washing buffer (Nacalai Tesque, Cat. L3P8406). Goat F(ab′)₂ anti-Human IgG, Mouse ads-PE (Southern Biotech, Cat. 2043-09) was then added and incubated for 30 minutes in the dark at 4 degrees C. and subsequently washed. Data acquisition was performed using LSR Fortessa X-20 (BD Biosciences) and analyzed with FlowJo software (Tree Star). FIG. 1 shows that all anti-HER2 S310F antibodies produced in Example 3, i.e., NMR0007, NMR0009, MHR0010, MHR0012, MHR014, MHR0015, MHR0016, MHR0017, MHR0018, MHR0019, MHR0060/MHR0060′, and MHR0082, bind to the FreeStyle 293 transfectant expressing HER2 S310F. FIG. 2 shows that anti-HER2 S310F antibodies such as NMR0009, MHR0010, MHR0016, MHR0019, and NMR0060/MHR0060′, bind to 5637 bladder cancer cell line with HER2 S310F. Binding activities of each antibody is represented by the Mean Fluorescence Intensity values (MFI).

### Example 5. Functional Evaluation of Anti-HER2 S310F/CD3 Bispecific Antibodies

### Example 5.1 Measurement of T Cell Activation Activity of Anti-HER2 S310F/CD3 Bispecific Antibodies

HER2 S310F monospecific antibodies such as NMR0009, MHR0010, MHR0016, MHR0019, and NMR0060 and an anti-CD3 antibody described in Table 3 were used to generate anti-HER2 S310F/CD3 bispecific antibodies using conventional methods published in WO2015046467A1 or Sci Rep. 2017 Apr. 3; 7:45839. The sequences of the HER2 S310F-binding arm in the anti-HER2 S310F/CD3 bispecific antibodies are shown in Table 3. The bispecific antibodies generated contain a silent Fc with attenuated affinity for the Fc gamma receptor.

TABLE 3
HER2 S310F-binding arm in the anti-HER2 S310F/CD3 bispecific antibodies

| | | | Antibody name | | | | |
|---|---|---|---|---|---|---|---|
| | | | MHR0009/ | MHR0010/ | MHR0016/ | MHR0019/ | MHR0060/ |
| | | | CD3 | CD3 | CD3 | CD3 | CD3 |
| anti-HER2 S310F arm originated from | | | MHR0009 | MHR0010 | MHR0016 | MHR0019 | MHR0060 |
| SEQ | anti-HER2 | HCDR1 | 30 | 31 | 35 | 38 | 39 |
| ID NOs: | S310F arm | HCDR2 | 42 | 43 | 47 | 50 | 51 |
| | | HCDR3 | 54 | 55 | 59 | 62 | 63 |
| | | Heavy chain | 6 | 7 | 11 | 14 | 15 |
| | | variable region | | | | | |

| Heavy chain | 135 | | | | |
|---|---|---|---|---|---|
| constant region | | | | | |
| LCDR1 | 66 | 67 | 71 | 74 | 75 |
| LCDR2 | 78 | 79 | 83 | 86 | 87 |
| LCDR3 | 90 | 91 | 95 | 98 | 99 |
| Light chain | 18 | 19 | 23 | 26 | 27 |
| variable region | | | | | |

| | | |
|---|---|---|
| | Light chain | 136 |
| | constant region | |
| anti-CD3 | HCDR1 | 103 |
| arm | HCDR2 | 104 |
| | HCDR3 | 105 |
| | Heavy chain | 101 |
| | variable region | |
| | Heavy chain | 137 |
| | constant region | |
| | LCDR1 | 106 |
| | LCDR2 | 107 |
| | LCDR3 | 108 |
| | Light chain | 102 |
| | variable region | |
| | Light chain | 138 |
| | constant region | |

The ant-CD3 antibody described is also used to generate control bispecific antibodies such as HER2-1/CD3 and HER2-2/CD3 and a KLH/CD3 bispecific antibody. The sequences of the wild-type TIER2-binding arm or KLH-binding arm in the control bispecific antibodies are shown in Table 4. The bispecific antibodies generated contain a silent Fc with attenuated affinity for the Fc gamma receptor.

TABLE 4
Wild-type HER2-binding arm or KLH-binding
arm in the control bispecific antibodies

| | | | HER2-1/ | HER2-2/ | KLH/ |
|---|---|---|---|---|---|
| Antibody name | | | CD3 | CD3 | CD3 |
| SEQ | anti-Wild-type | HCDR1 | 115 | 116 | 117 |
| ID | HER2-binding | HCDR2 | 118 | 119 | 120 |
| NOs: | arm or KLH- | HCDR3 | 121 | 122 | 123 |
| | binding arm | Heavy chain | 109 | 110 | 111 |
| | | variable region | | | |

| Heavy chain | 135 | | |
|---|---|---|---|
| constant region | | | |
| LCDR1 | 124 | 125 | 126 |
| LCDR2 | 127 | 128 | 129 |
| LCDR3 | 130 | 131 | 132 |
| Light chain | 112 | 113 | 114 |
| variable region | | | |

| | | |
|---|---|---|
| | Light chain | 138 |
| | constant region | |
| anti-CD3 arm | HCDR1 | 103 |
| | HCDR2 | 104 |
| | HCDR3 | 105 |
| | Heavy chain | 101 |
| | variable region | |
| | Heavy chain | 137 |
| | constant region | |
| | LCDR1 | 106 |
| | LCDR2 | 107 |
| | LCDR3 | 108 |
| | Light chain | 102 |
| | variable region | |
| | Light chain | 138 |
| | constant region | |

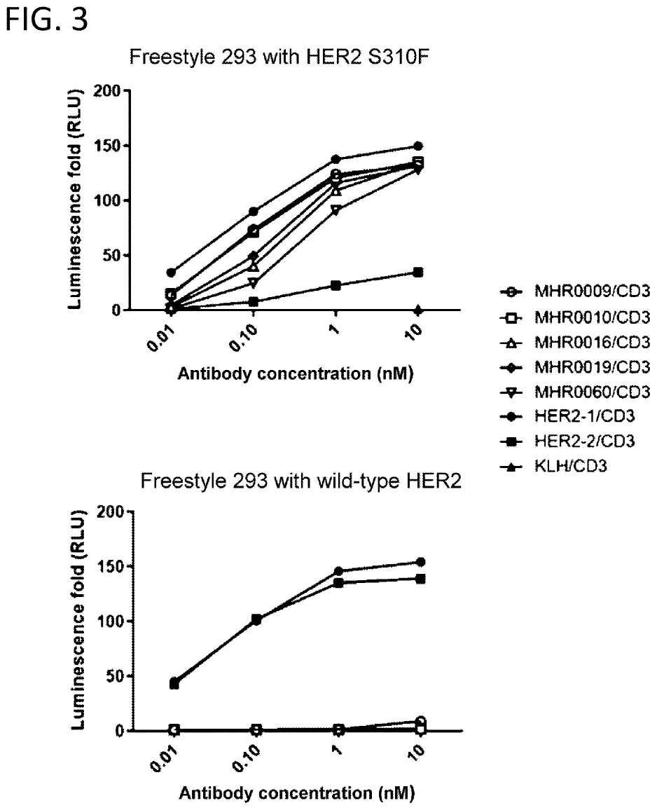

FIG. 3 shows T cell activation activity of anti-HER2 S310F/CD3 bispecific antibodies in the presence of FreeStyle 293 transfectant expressing HER2 S310F or FreeStyle 293 transfectant expressing wild-type ITER2 determined by luciferase assay system using GloResponse™ NFAT-luc2 Jurkat cells as effector cells. 20,000 target cells and 100,000 effector cells seeded (E/T=5) in 96-well white culture plates were incubated with varying concentrations of antibody for 24 hours at 37 degrees C. and 5% CO2. Luminescence (RLU) was measured using BIO-GLO® luciferase assay system (Promega, Cat. G7941) and T cell activation activity was calculated based on RLU fold change using the equation below.

$$RLU fold change = Antibody induced RLU / no antibody control induced RLU$$

Bispecific antibodies MHR0009/CD3, MHR0010/CD3, MHR0016/CD3, MHR0019/CD3, and MHR0060/CD3 induced T cell activation in the presence of FreeStyle 293 transfectant expressing HER2 S310F but not in transfectant expressing the wild-type HER2. That is, it was demonstrated that T cell activation induced by MHR0009/CD3, MHR0010/CD3, MHR0016/CD3, MHR0019/CD3, and MHR0060/CD3 is significantly specific to the HER2 S310F mutant.

### Example 5.2 Measurement of T-Cell-Dependent Cell Cytotoxicity of Anti-HER2 S310F/CD3 Bispecific Antibodies

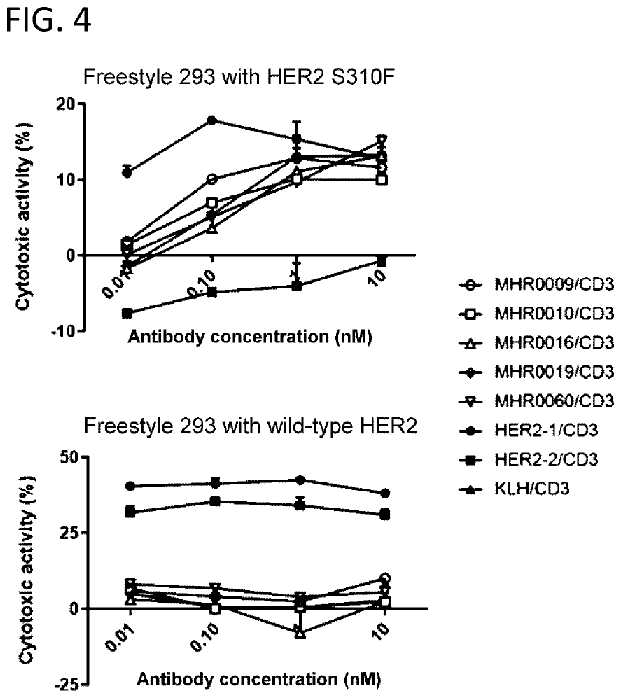

FIG. 4 shows the T-cell-dependent cell cytotoxicity of anti-HER2 S310F/CD3 bispecific antibodies against FreeStyle 293 transfectant expressing HER2 S310F or FreeStyle 293 transfectant expressing wild-type HER2. Cell cytotoxicity was evaluated by LDH assay using human PBMC (StemCell, Cat. 70025). 20,000 target cells and 200,000 human PBMC (E/T=10) were seeded into each well of a 96-well U-bottom plate and incubated with various concentrations of antibody for 24 hours at 37 degrees C. and 5% CO2. Target cell killing was measured by LDH cytotoxicity detection kit (Takara Bio). The cytotoxic activity (%) of each antibody was calculated using the following formula.

$$Cytotoxic activity ( \% ) = ( A - B - C ) \times 100 / ( D - C )$$

“A” represents the absorbance of wells treated with antibody and PBMC, “B” represents the average absorbance value of effector cells PBMC only, “C” represents the average absorbance value of untreated target cells only, and “D” represents the average values of wells lysed with Triton-X. Background absorbance have been accounted for and subtracted.

Bispecific antibodies MHR0009/CD3, MHR0010/CD3, MHR0016/CD3, MHR0019/CD3, and MHR0060/CD3 showed the cell cytotoxicity against FreeStyle 293 transfectant expressing HER2 S310F and did not induce the cell cytotoxicity against FreeStyle 293 transfectant expressing wild-type HER2. That is, MHR0009/CD3, MHR0010/CD3, MHR0016/CD3, MHR0019/CD3, and NMR0060/CD3 showed T cell-dependent cytotoxicity against FreeStyle 293 cells which is highly specific to the HER2 S310F mutant.

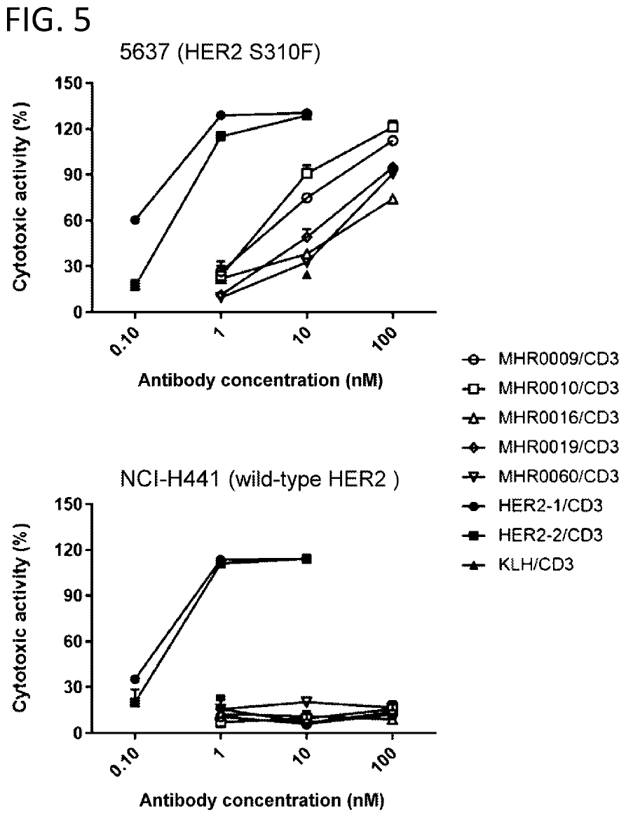

FIG. 5 shows the T-cell-dependent cell cytotoxicity of anti-HER2 S310F/CD3 bispecific antibodies against 5637 bladder cancer cell line with HER2 S310F (Proc Natl Acad Sci USA. 2012 Sep. 4; 109(36):14476-81) or NCI-H441 lung cancer cell line with wild-type HER2 as target cells. Cell cytotoxicity was assessed by the rate of cell growth inhibition using xCELLigence Real-Time Cell Analyzer (ACEA Biosciences). Target cells were seeded at desired density into E-plate 96 (ACEA Biosciences) to initiate the measurement of cell growth for 24 hours, after which, 50 microliters (micro L) each of respective test antibodies and expanded T-cells were added at an E/T ratio of 10. The effector T-cells used were isolated from Human PBMC (Stemcell, Cat. 70025) using a T-cell isolation kit (Stemcell, Cat. 17951) and activated using CD3/CD28 beads (Invitrogen, Cat. 1132D). Expanded T-cells were harvested at the end of 7 day culture. Cell growth was monitored for 92 hours after treatment at 37 degrees C. and 5% CO2. The Cell Growth Inhibition (CGI) rate (%) was determined using the equation below. The Cell Index Value obtained from xCELLigence Real-Time Cell Analyzer used in the calculation was a normalized value where the Cell Index value at the time point immediately before antibody addition was defined as 1.

$$CGI rate ( \% ) = ( A - B ) \times 100 / ( A - 1 )$$

A represents the mean Cell Index value in wells without antibody treatment (containing only target cells and human T cells), and B represents the mean Cell Index value of target wells. The examinations were performed in replicates.

Bispecific antibodies NMR0009/CD3, MHR0010/CD3, MHR0016/CD3, MHR0019/CD3, and MHR0060/CD3 showed the cytotoxicity against 5637 cell line (FIG. 5). In particular, MHR0009/CD3 and MHR0010/CD3 showed the strongest cytotoxicity against 5637 cell line. On the other hand, none of them did not show the cytotoxicity against the H441 cell line (FIG. 5). That is, it was demonstrated that the T cell-dependent cytotoxicity against bladder cancer cells exhibited by MHR0009/CD3, MHR0010/CD3, MHR0016/CD3, MHR0019/CD3, and NMR0060/CD3 is significantly specific to the HER2 S310F mutant.

### Example 5.3 Affinity Measurement of the Anti-HER2 S310F/CD3 Bispecific Antibodies

The binding affinity of anti-HER2 S310F/CD3 bispecific antibodies binding to human HER2 S310F were determined at 37 degrees C. using Biacore 8K instrument (GE Healthcare). Anti-human Fc (GE Healthcare) was immobilized onto all flow cells of a CM4 sensor chip using amine coupling kit (GE Healthcare). All antibodies and analytes were prepared in ACES pH 7.4 containing 20 mM ACES, 150 mM NaCl, 0.05% Tween 20, 0.005% NaN3. Each antibody was captured onto the sensor surface by anti-human Fc. Antibody capture levels were aimed at 200 resonance unit (RU). Recombinant human HER2 S310F was injected at 400 to 25 nM prepared by two-fold serial dilution, followed by dissociation. Sensor surface was regenerated each cycle with 3 M MgCl2. Binding affinity were determined by processing and fitting the data to 1:1 binding model using Biacore 8K Evaluation software (GE Healthcare). Binding specificity binding to wild-type HER2 was assessed as described above by injection of recombinant wild-type HER2 at 400 to 25 nM prepared by two-fold serial dilution. Binding affinity of anti-HER2 S310F/CD3 bispecific antibodies and wild type HER2/CD3 bispecific antibodies such as HER2-1/CD3 and HER2-2/CD3 as a control to recombinant HER2 S310F and wild-type HER2 are shown in Table 5.

TABLE 5
Affinity of the bispecific antibodies towards
human HER2 S310F or wild-type HER2

| | KD (M) | |
|---|---|---|
| Bispecific antibody name | HER2 S310F | Wild-type HER2 |
| HER2-1/CD3 | 2.12E−09 | 2.54E−09 |
| HER2-2/CD3 | n.d. | 5.50E−09 |
| MHR0009/CD3 | 3.62E−08 | n.d. |
| MHR0010/CD3 | 1.08E−08 | n.d. |
| MHR0016/CD3 | 2.23E−08 | n.d. |
| MHR0019/CD3 | 1.39E−08 | n.d. |
| MHR0060/CD3 | 4.04E−08 | n.d. |

In Table 5, “n.d.” means that the binding response was too low, and thus KD cannot be determined based on it. For MHR0009/CD3, MHR0010/CD3, MHR0016/CD3, MHR0019/CD3, and MHR0060/CD3, the KD values for wild-type HER2 cannot be calculated for this reason. This indicates that the specificity of these bispecific antibodies towards the HER2 S310F mutant is remarkably high.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

### INDUSTRIAL APPLICABILITY

The present invention provides antigen-binding molecules that show high specificity and binding activity against HER2 S310F, with low or no cross-reactivity against a wild type HER2. Such mutant specific anti-HER2 antigen-binding molecules could be developed as a promising agent that demonstrates superior specificity against tumor cells expressing the tumor-specific mutation (i.e. minimal off-target toxicity) for cancer therapy and/or diagnosis etc.

The present invention further provides novel multispecific antigen-binding molecules that have a strong anti-tumor activity and an excellent safety property of not inducing a cytokine storm or such independently from cancer antigens, and have long half-lives in blood. Cytotoxicity-inducing agents that comprise an antigen-binding molecule of the present invention as an active ingredient can target HER2 S310F-expressing cells and tumor tissues containing these cells and induce cell injury. Administration of a multispecific antigen-binding molecule of the present invention to patients makes it possible to have a desirable treatment that has not only a high level of safety but also a reduced physical burden, and is highly convenient.

1.-15. (canceled)

16. A method of treating cancer, comprising administering to an individual having a cancer expressing human HER2 with a S310F mutation (HER2 S310F) an effective amount of a polypeptide comprising a single means for binding human HER2 S310F and a CD3 antigen binding domain, wherein the means for binding human HER2 S310F does not bind to wild type human HER2.

17. The method of claim 16, wherein the cancer is bladder cancer, cervical cancer, colorectal cancer (CRC), non-small-cell lung cancer (NSCLC), esophagus cancer, head and neck cancer, skin cancer (melanoma), bile duct cancer, kidney cancer, stomach cancer, small intestine cancer, liver cancer, uterus cancer, duodenum cancer, breast cancer, or gall bladder cancer.

18. The method of claim 16, wherein the polypeptide prevents ligand-independent dimerization of human HER2 S310F.

19. The method of claim 16, wherein the polypeptide further comprises an Fc domain.

20. The method of claim 19, wherein the Fc domain has a reduced Fc gamma receptor-binding activity compared to a wild type Fc domain of IgG1.

21. The method of claim 16, wherein the CD3 antigen binding domain binds to human CD3 epsilon chain.
